(19) United States
(12) Patent Application Publication
(10) Pub. No.: US 2026/0212958 A1
(43) Pub. Date: Jul. 23, 2026

(54) MACHINE-LEARNING APPROACHES TO PAN-CANCER SCREENING IN WHOLE GENOME SEQUENCING

(71) Applicant: Laboratory Corporation of America Holdings, Burlington, NC (US)

(72) Inventor: Andrey Koch, San Diego, CA (US)

(73) Assignee: Laboratory Corporation of America Holdings, Burlington, NC (US)

(21) Appl. No.: 19/484,692

(22) PCT Filed: May 15, 2024
(86) PCT No.: PCT/US2024/029356

§ 371 Date: Nov. 14, 2025

Related U.S. Application Data
(60) Provisional application No. 63/502,289, filed on May 15, 2023.

(51) Int. Cl. G16B 40/20; G06N 20/00; G16B 20/10; G16B 30/00; G16H 50/20

(52) U.S. Cl. CPC G16B 40/20; G16B 20/10; G16B 30/00; G16H 50/20; G06N 20/00

(57) ABSTRACT

The present disclosure relates to machine learning techniques for pan-cancer screening in whole genome sequencing from non-invasive prenatal testing (NIPT) procedures. Particularly, aspects are directed to accessing NIPT sequence read data for a sample, where the NIPT sequence read data was generated as part of performing a whole genome sequencing assay on NIPT samples, and the NIPT sequence read data includes a bin count profile comprising sequence read counts for each bin associated with a segment of a reference genome, determining an indicator of systematic abnormality for the sample based on the sequence read data, classifying, using a machine learning model, the sample as being a negative class or positive class for cancer based on the sequence read data and the indicator of systematic abnormality, and outputting, using the machine learning model, the negative class or the positive class for cancer.

### CROSS-REFERENCE TO RELATED APPLICATION

The present application claims priority and benefit from U.S. Provisional Application No. 63/502,289, filed May 15, 2023, the entire contents of which are incorporated herein by reference for all purposes.

### FIELD

The present disclosure relates to pan-cancer screening, and in particular to machine learning techniques for pan-cancer screening in whole genome sequencing from non-invasive prenatal testing (NIPT) procedures.

### BACKGROUND

Traditional tissue biopsy in prenatal testing procedures involves taking a sample of placenta for analysis in highly invasive procedures such as genetic amniocentesis and chorionic villus sampling (CVS). Although the safety of the invasive procedures has improved greatly since their introduction, there remains a well-recognized risk of iatrogenic fetal loss. Newer approaches have been developed for prenatal testing procedures by analyzing fetal DNA within the blood stream of pregnant women as opposed to a tissue biopsy from the placenta. The fetus sheds DNA into the mother's bloodstream and clinical testing can thus detect if the fetus has genetic disorders by drawing blood from the mother (referred to as a liquid biopsy for Noninvasive Prenatal Testing (NIPT)). More specifically, circulating cell-free (ccf) fetal (ccff) DNA is present in the plasma of pregnant women. DNA of fetal origin ranges between 2% and 40% with a mean around 10% of the total ccf DNA across varying gestational ages. Unlike the invasive amniocentesis and CVS that present a low risk of iatrogenic fetal loss, liquid biopsy is completely safe in this regard while being noninvasive. By detecting small amounts of DNA from the placenta in a mother's bloodstream, NIPT can help identify if there is an increased chance for certain chromosome abnormalities that can affect a baby's health and development. It can also indicate if the mother is more likely to have a boy, girl or both. Many NIPT procedures screen for certain chromosomal abnormalities called trisomies. These include trisomy 21 (Down syndrome), trisomy 18 (Edwards syndrome) and trisomy 13 (Patau syndrome).

Next Generation Sequencing (NGS) has improved the flexibility and outcomes of NIPT procedures, providing highly sensitive and accurate high-throughput platforms for large-scale genomic testing. Whole-genome sequencing (WGS) is a comprehensive NGS method for analyzing entire genomes (translates all or a substantially all the 3 billion DNA base pairs that make up an entire genome by determining the order of the nucleotides (A, C, G, T)). The goal of whole-genome sequencing is, typically, to look for genetic aberrations (e.g., single nucleotide variants, deletions, insertions and copy number variants). Because the entire genome is being sequenced, changes in the noncoding sections of DNA within genes, called introns, can also be determined. Under normal conditions, introns are removed by RNA splicing during a post-transcriptional process, and alterations in these regions can be important to whether the DNA is transcribed into RNA or potentially result in a truncated, non-functional protein.

An alternative approach is to sequence only the exomes, called whole-exome sequencing (WES). Exomes are the part of the genome formed by exons, or coding regions, which when transcribed and translated become expressed into proteins. Exomes compose only about 2% of the whole genome. Because the genome is so much larger, exomes are able to be sequenced at a much greater depth (number of times a given nucleotide is sequenced) for lower cost. This greater depth provides more confidence in low frequency alterations. In contrast to WGS and WES, targeted genomic sequencing (TS) focuses on a panel of genes or targets known to have strong associations with pathogenesis of disease and/or clinical relevance, which means the depth can become even greater with reduced costs and data burden. This allows targeted sequencing to identify low frequency variants in targeted regions with high confidence, thus suitable for profiling low-quality and fragmented clinical DNA samples. However, WES and targeted panels only see part of the story as they focus on reduced areas of the genome. Consequently, for some research projects and genetics testing, WGS may be advantageous.

The cost of a WGS has decreased tremendously in recent years due to advances in next-generation sequencing technologies. Nevertheless, the cost of carrying out high-coverage NIPT using WGS can still be prohibitive. To overcome these challenges, low-coverage WGS (1× to 10×) and ultra-low coverage WGS (coverage below 1×) have been developed for NIPT. Coverage (or depth) in nucleic acid sequencing is the number of unique reads that include a given nucleotide in the reconstructed sequence. Low coverage sequencing refers to the general concept of aiming for a low number of unique reads of each region of a sequence. By sampling across the whole genome at a low depth, it is possible to reliably detect and predict common variants in samples. Low-coverage and ultra-low coverage WGS have been demonstrated to accurately assess common genetic variation. For example, the MaterniT® GENOME WGS test uses relatively low (~0.4×) sequencing coverage which is sufficient for detecting large subchromosomal and whole chromosomal events.

NGS of cell-free DNA (cfDNA) has also been demonstrated to be capable of detecting tumor-specific copy-number alterations in subjects with cancer. Cancer is a disease of the genome and is characterized by several types of alterations including point mutations, balanced and unbalanced chromosomal rearrangements, and copy-number alterations (CNA). Researchers have described the genomic profiles of tumor tissue obtained from numerous primary and metastatic tumors. In addition, the release of cfDNA by tumors into the bloodstream has been described for decades and has been more recently leveraged in both research and clinical settings to facilitate therapy selection, identify drug resistance, and monitor treatment response by detecting oncology signal through measuring genomic instability (e.g., measuring genomic instability number (GIN)). The GIN is a metric intended to capture genome-wide autosomal deviation from empirically derived euploid dosage of the genome in circulation. Depending on the breadth of the target genomic loci, this liquid biopsy process, is typically performed using digital polymerase chain reaction (PCR) or ultra-deep next-generation sequencing (>8,000-10,000×raw coverage). Although these assays are useful in guiding therapy through their detection of all types of mutations associated with cancer, cost factors currently necessitate these tests to narrow their content to alterations within a targeted region. In contrast as described above, detection of CNAs via low-coverage and ultra-low coverage WGS of cfDNA has been validated for NIPT where it has routinely been applied clinically. Because tumor aneuploidy (and thus the presence of CNAs) is a fundamental characteristic of cancer, NIPT procedures have on occasion detected CNAs in patients with either known or unknown neoplasms. Taking the approach of utilizing the information from the whole genome, disclosed herein are machine-learning techniques that take advantage of the utility of NIPT procedures to detect and monitor CNAs in patients with known cancers (pan-cancer screening). These machine-learning techniques demonstrate that the coverage level from low-coverage and ultra-low coverage WGS is adequate for oncology screening and can be used to advantageously keep the liquid biopsy test cost relatively low.

### SUMMARY

In various embodiments, a computer-implemented method is provided that comprises: accessing NIPT sequence read data for a first group samples, where the NIPT sequence read data was generated as part of performing a whole genome sequencing assay on NIPT samples, and the NIPT sequence read data includes bin count profiles comprising sequence read counts for each bin associated with a segment of a reference genome; generating a first subset of training data and a second subset of training data based on the NIPT sequence read data, where each example in the second subset of training data is classified as negative for cancer; accessing copy number variant data for a second group samples, where the copy number variant data was generated as part of performing a copy number variation assay, and the copy number variant data includes information on cancer patient derived genomic events, which are copy number counts for one or more segments that deviate from a reference profile; generating a set of synthetic training data based on the copy number variant data obtained from cancer patients, where generating the set of synthetic training data comprises generating an empirical population of the genomic events characteristic of the copy number variant data obtained from the cancer patients, extracting event features and standard scores from the empirical population of the genomic events, and mapping the event features and standard scores to the bin count profiles for the second subset of training data to generate the set of synthetic training data, and where each example in the set of synthetic training data is classified as positive for cancer; augmenting the first subset of training data with the set of synthetic training data to generate an augmented set of training data; and training, using the augmented set of training data, a machine learning model to classify a sample as being negative for cancer or positive for cancer, where the training comprises: iterative operations to find a set of parameters for the machine learning model that minimizes a loss or error function for the machine learning model, where each iteration includes finding the set of parameters for the machine learning model so that a value of the loss or error function using the set of parameters is smaller than a value of the loss or error function using another set of parameters in a previous iteration, wherein the loss or error function is configured to measure a difference between (i) class outputs inferred for each example in the augmented set of training data, and (ii) labels that provide ground truth information for each example in the augmented set of training data, and where the ground truth information identify whether the example is classified as negative for cancer or classified as positive for cancer.

In some embodiments, generating the first subset of training data and the second subset of training data comprises normalizing the NIPT sequence read data by: (i) smoothing the bin count profiles using LOESS, (ii) population-based correction of the bin count profiles using principal component analysis, or (iii) both.

In some embodiments, the computer-implemented method further comprises reducing dimensionality of the augmented set of training data using another principal component analysis, wherein the other principal component analysis comprises mapping the augmented set of training data on first k principal components while discriminating between negative and positive classes, which reduces data redundancy and reduces a feature space n.

In some embodiments, the computer-implemented method further comprises reducing dimensionality of the augmented set of training data using an onco-gene feature selection process, where the onco-gene feature selection process comprises generating a subset of predetermined number b of bins overlapped with a predetermined number o of onco-genes and use the bin count profiles from the normalized NIPT sequence read data at the subset of predetermined number b of bins as new model attributes.

In some embodiments, the computer-implemented method further comprises: determining an indicator of systematic abnormality in each example of the augmented set of training data, wherein the indicator is a genome instability number, a quantity of tumor content, or both; and appending the indicator of systematic abnormality to each respective example of augmented set of training data, where the machine learning model is trained, using the augmented set of training data with the appended indicator of systematic abnormality, to classify the sample as being negative for cancer or positive for cancer, and where the machine learning model uses the indicator of systematic abnormality as an additional model feature during training to find the set of parameters.

In some embodiments, the genome instability number is calculated as an integral of absolute deviations of a LOESS-smoothed normalized autosomal bin count profile from expectation.

In some embodiments, the set of synthetic training data presents a same statistical distribution of genomic events as presented in the copy number variant data.

In various embodiments, a computer-implemented method is provided that comprises: accessing NIPT sequence read data for a sample, wherein the sequence read data was generated as part of performing a whole genome sequencing assay on the sample, and the NIPT sequence read data includes a bin count profile comprising sequence read counts for each bin associated with a segment of a reference genome; determining an indicator of systematic abnormality for the sample based on the NIPT sequence read data, where the indicator is a genome instability number, a quantity of tumor content, or both; and inputting the NIPT sequence read data and the indicator of systematic abnormality into a machine learning model constructed as a binary classifier; classifying, using the machine learning model, the sample as being a negative class or positive class for cancer, where the machine learning model uses the indicator of systematic abnormality as an additional model feature for predicting the negative class or the positive class; and outputting, using the machine learning model, the negative class or the positive class for cancer.

In some embodiments, the machine learning model comprises a plurality of parameters trained using a set of augmented training data comprising: a set of original training data comprising NIPT sequence read data for a group of samples, where the NIPT sequence read data was generated as part of performing the whole genome sequencing assay on NIPT samples, and the NIPT sequence read data includes bin count profiles comprising sequence read counts for each bin associated with a segment of the reference genome; and a set of synthetic training data generated based on copy number variant data obtained from cancer patients, wherein the copy number variant data was generated as part of performing a copy number variation assay, and the copy number variant data includes information on cancer patient genomic events, which are copy number counts for one or more segments that deviate from a reference profile.

In some embodiments, the set of synthetic training data was generated by generating an empirical population of the genomic events characteristic of the copy number variant data obtained from the cancer patients, extracting event features and standard scores from the empirical population of the genomic events, and mapping the event features and standard scores to the bin count profiles for the second subset of training data to generate the set of synthetic training data, and wherein each example in the set of synthetic training data is classified as positive for cancer.

In some embodiments, the genome instability number is calculated as an integral of absolute deviations of a LOESS-smoothed normalized autosomal bin count profile from expectation.

In some embodiments, the computer-implemented method further comprises: generating a report that includes the negative class or the positive class for cancer and a result of the whole genome sequencing assay.

In some embodiments, the machine learning model is deployed on an invocable end point within a cloud infrastructure.

In some embodiments, the computer-implemented method further comprises invoking, using the invocable end point, the machine learning model via an application programming interface.

In some embodiments, the computer-implemented method further comprises providing a recommendation for the whole genome sequencing assay based on the classification for cancer.

In some embodiments, the computer-implemented method further comprises performing the whole genome sequencing assay on the sample from the subject to obtain an analytical result for a clinical diagnostic test.

In some embodiments, the computer-implemented method further comprising diagnosing and/or treating the subject based on the analytical result for the clinical diagnostic test and/or the classification for cancer.

In some embodiments, a system is provided that includes one or more data processors and a non-transitory computer readable medium containing instructions which, when executed on the one or more data processors, cause the one or more data processors to perform part or all of one or more methods or processes disclosed herein.

In some embodiments, a computer-program product is provided that is tangibly embodied in a non-transitory machine-readable medium and that includes instructions configured to cause one or more data processors to perform part or all of one or more methods disclosed herein.

The terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by embodiments and optional features, modification and variation of the concepts herein disclosed may be resorted to by those skilled in the art, and that such modifications and variations are considered to be within the scope of this invention as defined by the appended claims.

### BRIEF DESCRIPTION OF THE DRAWINGS

The present invention will be better understood in view of the following non-limiting figures, in which:

FIG. 1 shows a block diagram of a clinical test pipeline that implements a NIPT procedure or assay such as MaterniT® GENOME for analyzing nucleic acids in accordance with various embodiments.

FIG. 2 shows an overview of a pan-cancer screening approach that takes processed sequence read data (e.g., WGS sequence read data) from the clinical testing pipeline and implements a pan-cancer screening procedure or assay for predicting whether a subject does or does not have cancer in accordance with various embodiments.

FIG. 3 shows a block diagram illustrating a system for implementing techniques for preprocessing WGS sequence read data for subjects, using the pre-processed WGS sequence read data to train a machine learning model, and using the trained model to predict a classification of cancer for a subject from WGS sequence read data in accordance with various embodiments.

FIG. 4 shows a flowchart illustrating a process for training a machine learning model to predict a classification of cancer for a subject in accordance with various embodiments.

FIG. 5 shows a flowchart illustrating a process for using a machine learning model to predict a classification of cancer for a subject in accordance with various embodiments.

FIG. 6 shows a block diagram illustrating a computing environment in which various systems, methods, algorithms, machine learning models, and data structures may be implemented in accordance with various embodiments.

FIG. 7 shows a bin count augmentation mapping function in accordance with various embodiments.

FIGS. 8A and 8B show a comparison of event characteristic distributions in accordance with various embodiments.

FIGS. 9A-9D show the start position of a 1st event on a chromosome for both the real oncology and simulated oncology data in accordance with various embodiments.

FIGS. 10A-10B show event lengths on chromosomes with 1 and 2 events in accordance with various embodiments.

FIGS. 11A-11D show event lengths on chromosomes with >=3 events in accordance with various embodiments.

FIGS. 12A-12C show gaps between events on a single chromosome in accordance with various embodiments.

FIGS. 13A-13D show SPCA standard deviation conditional on event length in accordance with various embodiments.

FIGS. 14A-14D show a comparison of event characteristic distributions in accordance with various embodiments.

FIG. 15A shows the machine learning results of all positive and negative samples of MaterniT GENOME in accordance with various embodiments.

FIG. 15B shows the machine learning results of only negative samples of MaterniT GENOME in accordance with various embodiments.

In the appended figures, similar components and/or features can have the same reference label. Further, various components of the same type can be distinguished by following the reference label by a dash and a second label that distinguishes among the similar components. If only the first reference label is used in the specification, the description is applicable to any one of the similar components having the same first reference label irrespective of the second reference label.

### DETAILED DESCRIPTION

The ensuing description provides preferred exemplary embodiments only, and is not intended to limit the scope, applicability or configuration of the disclosure. Rather, the ensuing description of the preferred exemplary embodiments will provide those skilled in the art with an enabling description for implementing various embodiments. It is understood that various changes may be made in the function and arrangement of elements without departing from the spirit and scope as set forth in the appended claims.

Specific details are given in the following description to provide a thorough understanding of the embodiments. However, it will be understood that the embodiments may be practiced without these specific details. For example, circuits, systems, networks, processes, and other components may be shown as components in block diagram form in order not to obscure the embodiments in unnecessary detail. In other instances, well-known circuits, processes, algorithms, structures, and techniques may be shown without unnecessary detail in order to avoid obscuring the embodiments.

Also, it is noted that individual embodiments may be described as a process which is depicted as a flowchart, a flow diagram, a data flow diagram, a structure diagram, or a block diagram. Although a flowchart or diagram may describe the operations as a sequential process, many of the operations may be performed in parallel or concurrently. In addition, the order of the operations may be re-arranged. A process is terminated when its operations are completed, but could have additional steps not included in a figure. A process may correspond to a method, a function, a procedure, a subroutine, a subprogram, etc. When a process corresponds to a function, its termination may correspond to a return of the function to the calling function or the main function.

### I. INTRODUCTION

MaterniT® GENOME is one of Sequenom's blood-based NIPT tests that predicts genetic syndromes in a fetus from information in fetal DNA. Chromosomes are how cells transfer genetic information as a baby develops, and extra or missing parts of chromosomes, or whole chromosome changes, can severely impact the health of a baby. Most NIPTs analyze information from select chromosomes, and like these NIPTs, MaterniT® GENOME can screen for trisomies 21 (Down syndrome), 18 (Edwards syndrome), and 13 (Patau syndrome), and whether a mother is having a boy or a girl. However, changes can be found in all chromosomes- and MaterniT® GENOME has been developed to analyze them all using WGS and displays both mother and fetus DNA profiles across the entire human genome. From these NIPTs, it has been found by Sequenom that some mothers tested using MaterniT® GENOME display an abnormal genomic profile, not associated with fetus DNA, that might indicate undiagnosed cancer. These findings align with the general understanding in the field of genomic testing that NIPT for fetal aneuploidy screening using cell-free DNA derived from maternal plasma can incidentally raise suspicion for cancer. Thus, it is hypothesized that sequencing data typical of NIPT testing (including WGS used in the MaterniT® GENOME) carries a hidden cancer signal. The challenge is how to extract that signal in a cost-effective manner with sufficient specificity and sensitivity.

This signal has been conventionally extracted either by an expert in the field such as a Lab Director based on the Classification Report summarizing WGS findings or using a statistical measure of abnormality such as the GIN. GIN is an indicator of the systematic abnormality in a patient genomic composition and calculated as the integral of absolute deviations of the LOESS-smoothed normalized autosomal bin count profile from expectation. More specifically, genome profile variability is smoothed with a LOESS fit for each chromosome, and absolute deviations of LOESS fit from expectation are summed over to calculate an AUC metric-GIN. An empirical threshold is used to predict a sample oncology status. The method is described in further detail in Jensen T J, Goodman A M, Kato S, Ellison C K, Daniels G A, Kim L, Nakashe P, McCarthy E, Mazloom A R, McLennan G, Grosu D S, Ehrich M, Kurzrock R. Genome-Wide Sequencing of Cell-Free DNA Identifies Copy-Number Alterations That Can Be Used for Monitoring Response to Immunotherapy in Cancer Patients. Mol Cancer Ther. 2019 February; 18 (2): 448-458. doi: 10.1158/1535-7163.MCT-18-0535. Epub 2018 Dec. 6. PMID: 30523049, the entire content of which is incorporated herein by reference for all purposes.

Nonetheless, expert analysis and statistical measures of abnormality such as GIN typically demonstrate low sensitivity (capability of classifying an individual as having cancer) especially at early stages of cancer (e.g., the GIN method sensitivity by itself is typically <60% and elevated in patients at later disease stages). It is hypothesized that this is because expert analysis and statistical measures alone or in combination are incapable of exploring all features or attributes that may or may not be associated with different pathological or physiological states. In order to overcome this challenge and others, aspects of the present disclosure are directed to machine learning (ML) techniques that are capable of exploring variation between WGS profiles and uncovering previously unknow features or attributes that form distinct patterns that could be associated with different pathological or physiological states.

ML has had tremendous impacts on numerous areas of modern society. For example, it is used for filtering spam messages from text documents, such as e-mail, analyzing various images to distinguish differences, and extraction of important data from large datasets through data mining. ML makes it possible to uncover patterns, construct models, and make predictions by learning from training data. ML algorithms are used in a broad range of domains, including biology and genomics. Deep learning (DL) is a subset of ML that differs from other ML processes in many ways. Most ML models perform well due to their custom-designed representation and input features. Using the input data generated through that process, ML learns algorithms, optimizes the weights of each feature, and optimizes the final prediction. DL attempts to learn multiple levels of representation using a hierarchy of multiple layers. In recent years, DL has overtaken ML in many areas, including speech, vision, and natural language processing. DL and ML are also increasingly used in the medical field, mainly in the areas of image analysis, drug research and development, data mining from medical documents, and speech. In addition to image and text data from medical charts generated in hospitals, various types of laboratory data including sequencing data may also be analyzed to detect various signals associated with the health of a subject. The main objective supporting the present disclosure was to develop an accurate and inexpensive machine learning approach for oncology detection in an NIPT setting that will provide increased sensitivity compared to other methods and will maintain a low computational burden. The method will utilize the data (e.g., WGS data) from NIPT procedures or assays such as the MaterniT® GENOME test pipeline and will present a separate clinical test/assay in addition to the existing one.

In an illustrative embodiment, a computer-implemented method is provided that comprises: accessing NIPT sequence read data for a first group samples, where the NIPT sequence read data was generated as part of performing a whole genome sequencing assay on NIPT samples, and the NIPT sequence read data includes bin count profiles comprising sequence read counts for each bin associated with a segment of a reference genome; generating a first subset of training data and a second subset of training data based on the NIPT sequence read data, where each example in the second subset of training data is classified as negative for cancer; accessing copy number variant data for a second group samples, where the copy number variant data was generated as part of performing a copy number variation assay, and the copy number variant data includes information on cancer patient derived genomic events, which are copy number counts for one or more segments that deviate from a reference profile; generating a set of synthetic training data based on the copy number variant data obtained from cancer patients, where generating the set of synthetic training data comprises generating an empirical population of the genomic events characteristic of the copy number variant data obtained from the cancer patients, extracting event features and standard scores from the empirical population of the genomic events, and mapping the event features and standard scores to the bin count profiles for the second subset of training data to generate the set of synthetic training data, and where each example in the set of synthetic training data is classified as positive for cancer; augmenting the first subset of training data with the set of synthetic training data to generate an augmented set of training data; and training, using the augmented set of training data, a machine learning model to classify a sample as being negative for cancer or positive for cancer, where the training comprises: iterative operations to find a set of parameters for the machine learning model that minimizes a loss or error function for the machine learning model, where each iteration includes finding the set of parameters for the machine learning model so that a value of the loss or error function using the set of parameters is smaller than a value of the loss or error function using another set of parameters in a previous iteration, wherein the loss or error function is configured to measure a difference between (i) class outputs inferred for each example in the augmented set of training data, and (ii) labels that provide ground truth information for each example in the augmented set of training data, and where the ground truth information identify whether the example is classified as negative for cancer or classified as positive for cancer.

In another illustrative embodiment, a computer-implemented method is provided that comprises: accessing NIPT sequence read data for a sample, wherein the sequence read data was generated as part of performing a whole genome sequencing assay on the sample, and the NIPT sequence read data includes a bin count profile comprising sequence read counts for each bin associated with a segment of a reference genome; determining an indicator of systematic abnormality for the sample based on the NIPT sequence read data, where the indicator is a genome instability number, a quantity of tumor content, or both; and inputting the NIPT sequence read data and the indicator of systematic abnormality into a machine learning model constructed as a binary classifier; classifying, using the machine learning model, the sample as being a negative class or positive class for cancer, where the machine learning model uses the indicator of systematic abnormality as an additional model feature for predicting the negative class or the positive class; and outputting, using the machine learning model, the negative class or the positive class for cancer.

As used herein, the terms “substantially,” “approximately” and “about” are defined as being largely but not necessarily wholly what is specified (and include wholly what is specified) as understood by one of ordinary skill in the art. In any disclosed embodiment, the term “substantially,” “approximately,” or “about” may be substituted with “within [a percentage] of” what is specified, where the percentage includes 0.1, 1, 5, and 10 percent. As used herein, when an action is “based on” something, this means the action is based at least in part on at least a part of the something.

### II. NIPT PIPELINE

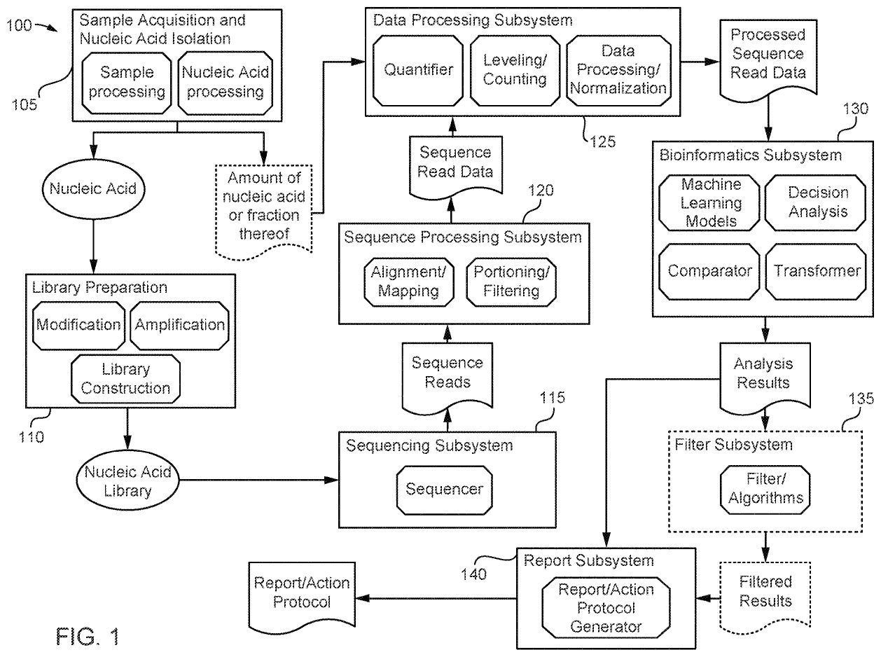

FIG. 1 is a block diagram of a clinical test pipeline 100 that implements a NIPT procedure or assay such as MaterniT® GENOME for analyzing nucleic acids. In some embodiments, nucleic acid fragments in a mixture of nucleic acid fragments are analyzed. Nucleic acid fragments may be referred to as nucleic acid templates, and the terms may be used interchangeably herein. A mixture of nucleic acids can comprise two or more nucleic acid fragment species having the same or different nucleotide sequences, different fragment lengths, different origins (e.g., genomic origins, fetal vs. maternal origins, cell or tissue origins, cancer vs. non-cancer origin, tumor vs. non-tumor origin, sample origins, subject origins, and the like), or combinations thereof.

The terms “nucleic acid,” “nucleic acid molecule,” “nucleic acid fragment,” and “nucleic acid template” may be used interchangeably throughout the disclosure. The terms refer to nucleic acids of any composition from, such as DNA (e.g., complementary DNA (cDNA), genomic DNA (gDNA) and the like), RNA (e.g., message RNA (mRNA), short inhibitory RNA (siRNA), ribosomal RNA (rRNA), tRNA, microRNA, RNA highly expressed by a fetus or placenta, and the like), and/or DNA or RNA analogs (e.g., containing base analogs, sugar analogs and/or a non-native backbone and the like), RNA/DNA hybrids and polyamide nucleic acids (PNAs), all of which can be in single- or double-stranded form, and unless otherwise limited, can encompass known analogs of natural nucleotides that can function in a similar manner as naturally occurring nucleotides. A nucleic acid may be, or may be from, a plasmid, phage, virus, bacterium, autonomously replicating sequence (ARS), mitochondria, centromere, artificial chromosome, chromosome, or other nucleic acid able to replicate or be replicated in vitro or in a host cell, a cell, a cell nucleus or cytoplasm of a cell in certain embodiments. A template nucleic acid in some embodiments can be from a single chromosome (e.g., a nucleic acid sample may be from one chromosome of a sample obtained from a diploid organism). Unless specifically limited, the term encompasses nucleic acids containing known analogs of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, single nucleotide polymorphisms (SNPs), and complementary sequences as well as the sequence explicitly indicated. Specifically, degenerate codon substitutions may be achieved by generating sequences in which the third position of one or more selected (or all) codons is substituted with mixed-base and/or deoxyinosine residues. The term nucleic acid is used interchangeably with locus, gene, cDNA, and mRNA encoded by a gene. The term also may include, as equivalents, derivatives, variants and analogs of RNA or DNA synthesized from nucleotide analogs, single-stranded (“sense” or “antisense,” “plus” strand or “minus” strand, “forward” reading frame or “reverse” reading frame) and double-stranded polynucleotides. The term “gene” refers to a section of DNA involved in producing a polypeptide chain; and generally includes regions preceding and following the coding region (leader and trailer) involved in the transcription/translation of the gene product and the regulation of the transcription/translation, as well as intervening sequences (introns) between individual coding regions (exons). A nucleotide or base generally refers to the purine and pyrimidine molecular units of nucleic acid (e.g., adenine (A), thymine (T), guanine (G), and cytosine (C)). For RNA, the base thymine is replaced with uracil. Nucleic acid length or size may be expressed as a number of bases.

At block 105, nucleic acid or a nucleic acid mixture utilized in systems, methods and products described herein may be isolated from a sample obtained from a subject (e.g., a test subject). In some embodiments, nucleic acid is extracted from cells using a cell lysis procedure. Cell lysis procedures and reagents are known in the art and may generally be performed by chemical (e.g., detergent, hypotonic solutions, enzymatic procedures, and the like, or combination thereof), physical (e.g., French press, sonication, and the like), or electrolytic lysis methods. Any suitable lysis procedure can be utilized. For example, chemical methods generally employ lysing agents to disrupt cells and extract the nucleic acids from the cells, followed by treatment with chaotropic salts. Physical methods such as freeze/thaw followed by grinding, the use of cell presses and the like also are useful. In some instances, a high salt and/or an alkaline lysis procedure may be utilized.

In some embodiments, nucleic acids can include extracellular nucleic acid. The term “extracellular nucleic acid” as used herein can refer to nucleic acid isolated from a source having substantially no cells and also is referred to as “cell-free” nucleic acid, “circulating cell-free nucleic acid” (e.g., CCF fragments, ccf DNA) and/or “cell-free circulating nucleic acid.” Extracellular nucleic acid can be present in and obtained from blood (e.g., from the blood of a human subject). Extracellular nucleic acid often includes no detectable cells and may contain cellular elements or cellular remnants. Non-limiting examples of acellular sources for extracellular nucleic acid are blood, blood plasma, blood serum and urine. As used herein, the term “obtain cell-free circulating sample nucleic acid” includes obtaining a sample directly (e.g., collecting a sample, e.g., a test sample) or obtaining a sample from another who has collected a sample. Without being limited by theory, extracellular nucleic acid may be a product of cell apoptosis and cell breakdown, which provides basis for extracellular nucleic acid often having a series of lengths across a spectrum (e.g., a “ladder”). In some embodiments, sample nucleic acid from a test subject is circulating cell-free nucleic acid. In some embodiments, circulating cell free nucleic acid is from blood plasma or blood serum from a test subject.

A subject can be any living or non-living organism, including but not limited to a human, a non-human animal, a plant, a bacterium, a fungus, a protest or a pathogen. Any human or non-human animal can be selected, and may include, for example, mammal, reptile, avian, amphibian, fish, ungulate, ruminant, bovine (e.g., cattle), equine (e.g., horse), caprine and ovine (e.g., sheep, goat), swine (e.g., pig), camelid (e.g., camel, llama, alpaca), monkey, ape (e.g., gorilla, chimpanzee), ursid (e.g., bear), poultry, dog, cat, mouse, rat, fish, dolphin, whale and shark. A subject may be a male or female (e.g., woman, a pregnant woman). A subject may be any age (e.g., an embryo, a fetus, an infant, a child, an adult). A subject may be a cancer patient, a patient suspected of having cancer, a patient in remission, a patient with a family history of cancer, and/or a subject obtaining a cancer screen.

Nucleic acid may be isolated from any type of suitable biological specimen or sample (e.g., a test sample). A sample or test sample can be any specimen that is isolated or obtained from a subject or part thereof (e.g., a human subject, a pregnant female, a cancer patient, a fetus, a tumor). A sample sometimes is from a pregnant female subject bearing a fetus at any stage of gestation (e.g., first, second or third trimester for a human subject), and sometimes is from a post-natal subject. A sample sometimes is from a pregnant subject bearing a fetus that is euploid for all chromosomes, and sometimes is from a pregnant subject bearing a fetus having a chromosome aneuploidy (e.g., one, three (i.e., trisomy (e.g., T21, T18, T13)), or four copies of a chromosome) or other genetic variation. Non-limiting examples of specimens include fluid or tissue from a subject, including, without limitation, blood or a blood product (e.g., serum, plasma, or the like), umbilical cord blood, chorionic villi, amniotic fluid, cerebrospinal fluid, spinal fluid, lavage fluid (e.g., bronchoalveolar, gastric, peritoneal, ductal, ear, arthroscopic), biopsy sample (e.g., from pre-implantation embryo; cancer biopsy), celocentesis sample, cells (blood cells, placental cells, embryo or fetal cells, fetal nucleated cells or fetal cellular remnants, normal cells, abnormal cells (e.g., cancer cells)) or parts thereof (e.g., mitochondrial, nucleus, extracts, or the like), washings of female reproductive tract, urine, feces, sputum, saliva, nasal mucous, prostate fluid, lavage, semen, lymphatic fluid, bile, tears, sweat, breast milk, breast fluid, the like or combinations thereof. In some embodiments, a biological sample is a cervical swab from a subject. A fluid or tissue sample from which nucleic acid is extracted may be acellular (e.g., cell-free). In some embodiments, a fluid or tissue sample may contain cellular elements or cellular remnants. In some embodiments, fetal cells or cancer cells may be included in the sample.

A sample can be a liquid sample. A liquid sample can comprise extracellular nucleic acid (e.g., circulating cell-free DNA). Non-limiting examples of liquid samples, include, blood or a blood product (e.g., serum, plasma, or the like), urine, biopsy sample (e.g., liquid biopsy for the detection of cancer), a liquid sample described above, the like or combinations thereof. In certain embodiments, a sample is a liquid biopsy, which generally refers to an assessment of a liquid sample from a subject for the presence, absence, progression or remission of a disease (e.g., cancer). A liquid biopsy can be used in conjunction with, or as an alternative to, a sold biopsy (e.g., tumor biopsy). In certain instances, extracellular nucleic acid is analyzed in a liquid biopsy.

In some embodiments, a biological sample may be blood, plasma or serum. The term “blood” encompasses whole blood, blood product or any fraction of blood, such as serum, plasma, buffy coat, or the like as conventionally defined. Blood or fractions thereof often comprise nucleosomes. Nucleosomes comprise nucleic acids and are sometimes cell-free or intracellular. Blood also comprises buffy coats. Buffy coats are sometimes isolated by utilizing a ficoll gradient. Buffy coats can comprise white blood cells (e.g., leukocytes, T-cells, B-cells, platelets, and the like). Blood plasma refers to the fraction of whole blood resulting from centrifugation of blood treated with anticoagulants. Blood serum refers to the watery portion of fluid remaining after a blood sample has coagulated. Fluid or tissue samples often are collected in accordance with standard protocols hospitals or clinics generally follow. For blood, an appropriate amount of peripheral blood (e.g., between 3 to 40 milliliters, between 5 to 50 milliliters) often is collected and can be stored according to standard procedures prior to or after preparation.

An analysis of nucleic acid found in a subject's blood may be performed using, e.g., whole blood, serum, or plasma. An analysis of fetal DNA found in maternal blood, for example, may be performed using, e.g., whole blood, serum, or plasma. An analysis of tumor DNA found in a patient's blood, for example, may be performed using, e.g., whole blood, serum, or plasma. Methods for preparing serum or plasma from blood obtained from a subject (e.g., a maternal subject; cancer patient) are known. For example, a subject's blood (e.g., a pregnant woman's blood; cancer patient's blood) can be placed in a tube containing EDTA or a specialized commercial product such as Vacutainer SST (Becton Dickinson, Franklin Lakes, N.J.) to prevent blood clotting, and plasma can then be obtained from whole blood through centrifugation. Serum may be obtained with or without centrifugation-following blood clotting. If centrifugation is used then it is typically, though not exclusively, conducted at an appropriate speed, e.g., 1,500-3,000 times g. Plasma or serum may be subjected to additional centrifugation steps before being transferred to a fresh tube for nucleic acid extraction. In addition to the acellular portion of the whole blood, nucleic acid may also be recovered from the cellular fraction, enriched in the buffy coat portion, which can be obtained following centrifugation of a whole blood sample from the subject and removal of the plasma.

In some embodiments, the nucleic acid or the nucleic acid mixture (e.g., extracellular nucleic acid) is enriched or relatively enriched for a subpopulation or species of nucleic acid. Nucleic acid subpopulations can include, for example, fetal nucleic acid, maternal nucleic acid, cancer nucleic acid, patient nucleic acid, nucleic acid comprising fragments of a particular length or range of lengths, or nucleic acid from a particular genome region (e.g., single chromosome, set of chromosomes, and/or certain chromosome regions). Such enriched samples can be used in conjunction with a method provided herein. Thus, in certain embodiments, methods of the technology comprise an additional step of enriching for a subpopulation of nucleic acid in a sample, such as, for example, cancer or fetal nucleic acid. In certain embodiments, a method for determining fraction of cancer cell nucleic acid or fetal fraction also can be used to enrich for cancer or fetal nucleic acid. In certain embodiments, nucleic acid from normal tissue (e.g., non-cancer cells) is selectively removed (partially, substantially, almost completely or completely) from the sample. In certain embodiments, maternal nucleic acid is selectively removed (partially, substantially, almost completely or completely) from the sample. In certain embodiments, enriching for a particular low copy number species nucleic acid (e.g., cancer or fetal nucleic acid) may improve quantitative sensitivity. Methods for enriching a sample for a particular species of nucleic acid are described, for example, in U.S. Pat. No. 6,927,028, International Patent Application Publication No. WO 2007/140417, International Patent Application Publication No. WO 2007/147063, International Patent Application Publication No. WO 2009/032779, International Patent Application Publication No. WO 2009/032781, International Patent Application Publication No. WO 2010/033639, International Patent Application Publication No. WO 2011/034631, International Patent Application Publication No. WO 2006/056480, and International Patent Application Publication No. WO 2011/143659, the entire content of each is incorporated herein by reference for all purposes, including all text, tables, equations and drawings, for all purposes.

The amount of nucleic acid (e.g., concentration, relative amount, absolute amount, copy number, and the like) in a sample may be determined. The amount of a minority nucleic acid (e.g., concentration, relative amount, absolute amount, copy number, and the like) in nucleic acid is determined in some embodiments. In certain embodiments, the amount of a minority nucleic acid species in a sample is referred to as “minority species fraction.” In some embodiments, “minority species fraction” refers to the fraction of a minority nucleic acid species in circulating cell-free nucleic acid in a sample (e.g., a blood sample, a serum sample, a plasma sample, a urine sample) obtained from a subject. The amount of a minority nucleic acid in extracellular nucleic acid can be quantified and used in conjunction with a method provided herein. Thus, in certain embodiments, methods described herein comprise an additional step of determining the amount of a minority nucleic acid. The amount of a minority nucleic acid can be determined in a sample from a subject before or after processing to prepare sample nucleic acid. In certain embodiments, the amount of a minority nucleic acid is determined in a sample after sample nucleic acid is processed and prepared, which amount is utilized for further assessment. In some embodiments, an outcome comprises factoring the minority species fraction in the sample nucleic acid (e.g., adjusting counts, removing samples, making a call or not making a call).

A determination of minority species fraction can be performed before, during, or at any one point in clinical test pipeline 100 described herein, or after certain processes described herein (e.g., detection of a genetic variation or genetic alteration). For example, to conduct a genetic variation/genetic alteration determination method with a certain sensitivity or specificity, a minority nucleic acid quantification method may be implemented prior to, during or after genetic variation/genetic alteration determination to identify those samples with greater than about 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25% or more minority nucleic acid. In some embodiments, samples determined as having a certain threshold amount of minority nucleic acid (e.g., about 15% or more minority nucleic acid; about 4% or more minority nucleic acid) are further analyzed for a genetic variation/genetic alteration, or the presence or absence of a genetic variation/genetic alteration, for example. In certain embodiments, determinations of, for example, a genetic variation or genetic alteration are selected (e.g., selected and communicated to a patient) only for samples having a certain threshold amount of a minority nucleic acid (e.g., about 15% or more minority nucleic acid; about 4% or more minority nucleic acid).

For example, the amount of cancer cell nucleic acid (e.g., concentration, relative amount, absolute amount, copy number, and the like) in nucleic acid may be determined. In certain instances, the amount of cancer cell nucleic acid in a sample is referred to as “fraction of cancer cell nucleic acid,” and sometimes is referred to as “cancer fraction” or “tumor fraction.” In some embodiments, “fraction of cancer cell nucleic acid” refers to the fraction of cancer cell nucleic acid in circulating cell-free nucleic acid in a sample (e.g., a blood sample, a serum sample, a plasma sample, a urine sample) obtained from a subject. Additionally or alternatively, the amount of fetal nucleic acid (e.g., concentration, relative amount, absolute amount, copy number, and the like) in nucleic acid may be determined. In certain embodiments, the amount of fetal nucleic acid in a sample is referred to as “fetal fraction.” In some embodiments, “fetal fraction” refers to the fraction of fetal nucleic acid in circulating cell-free nucleic acid in a sample (e.g., a blood sample, a serum sample, a plasma sample, a urine sample) obtained from a pregnant female. Certain methods described herein or known in the art for determining fetal fraction can be used for determining a fraction of cancer cell nucleic acid and/or a minority species fraction.

In certain instances, fetal fraction may be determined according to markers specific to a male fetus (e.g., Y-chromosome STR markers (e.g., DYS 19, DYS 385, DYS 392 markers); RhD marker in RhD-negative females), allelic ratios of polymorphic sequences, or according to one or more markers specific to fetal nucleic acid and not maternal nucleic acid (e.g., differential epigenetic biomarkers (e.g., methylation) between mother and fetus, or fetal RNA markers in maternal blood plasma (see e.g., Lo, 2005, Journal of Histochemistry and Cytochemistry 53 (3): 293-296)). Determination of fetal fraction sometimes is performed using a fetal quantifier assay (FQA) as described, for example, in U.S. Patent Application Publication No. 2010/0105049, the entire content of which is incorporated herein by reference for all purposes. This type of assay allows for the detection and quantification of fetal nucleic acid in a maternal sample based on the methylation status of the nucleic acid in the sample.

In certain embodiments, a minority species fraction can be determined based on allelic ratios of polymorphic sequences (e.g., single nucleotide polymorphisms (SNPs)), such as, for example, using a method described in U.S. Patent Application Publication No. 2011/0224087, the entire content of which is incorporated herein by reference for all purposes. In such a method for determining fetal fraction, for example, nucleotide sequence reads are obtained for a maternal sample and fetal fraction is determined by comparing the total number of nucleotide sequence reads that map to a first allele and the total number of nucleotide sequence reads that map to a second allele at an informative polymorphic site (e.g., SNP) in a reference genome.

A minority species fraction can be determined, in some embodiments, using methods that incorporate information derived from chromosomal aberrations as described, for example, in International Patent Application Publication No. WO2014/055774, the entire content of which is incorporated herein by reference for all purposes. A minority species fraction can be determined, in some embodiments, using methods that incorporate information derived from sex chromosomes as described, for example, in U.S. Patent Application Publication No. 2013/0288244 and U.S. Patent Application Publication No. 2013/0338933, the entire content of which is incorporated herein by reference for all purposes.

A minority species fraction can be determined in some embodiments using methods that incorporate fragment length information (e.g., fragment length ratio (FLR) analysis, fetal ratio statistic (FRS) analysis as described in International Patent Application Publication No. WO 2013/177086, the entire content of which is incorporated herein by reference for all purposes). Cell-free fetal nucleic acid fragments generally are shorter than maternally-derived nucleic acid fragments (see e.g., Chan et al. (2004) Clin. Chem. 50:88-92; Lo et al. (2010) Sci. Transl. Med. 2: 61ra91). Thus, fetal fraction can be determined, in some embodiments, by counting fragments under a particular length threshold and comparing the counts, for example, to counts from fragments over a particular length threshold and/or to the amount of total nucleic acid in the sample. Methods for counting nucleic acid fragments of a particular length are described in further detail in International Patent Application Publication No. WO 2013/177086.

A minority species fraction can be determined, in some embodiments, according to portion-specific fraction estimates (e.g., as described in International Patent Application Publication No. WO 2014/205401, the entire content of which is incorporated herein by reference for all purposes). Without being limited to theory, the amount of reads from fetal CCF fragments (e.g., fragments of a particular length, or range of lengths) often map with ranging frequencies to portions (e.g., within the same sample, e.g., within the same sequencing run). Also, without being limited to theory, certain portions, when compared among multiple samples, tend to have a similar representation of reads from fetal CCF fragments (e.g., fragments of a particular length, or range of lengths), and that the representation correlates with portion-specific fetal fractions (e.g., the relative amount, percentage or ratio of CCF fragments originating from a fetus). Portion-specific fetal fraction estimates generally are determined according to portion-specific parameters and their relation to fetal fraction.

In some embodiments, the determination of minority species fraction (e.g., fraction of cancer cell nucleic acid; fetal fraction) is not required or necessary for identifying the presence or absence of a genetic variation or genetic alteration. In some embodiments, identifying the presence or absence of a genetic variation or genetic alteration does not require a sequence differentiation of a minority nucleic acid versus a majority nucleic acid. In certain embodiments, this is because the summed contribution of both minority and majority sequences in a particular chromosome, chromosome portion or part thereof is analyzed. In some embodiments, identifying the presence or absence of a genetic variation or genetic alteration does not rely on a priori sequence information that would distinguish minority nucleic acid from majority nucleic acid.

At block 110, a nucleic acid library is prepared from the isolated nucleic acid or a nucleic acid mixture. In some embodiments, a nucleic acid library is a plurality of polynucleotide molecules (e.g., a sample of nucleic acids) that are prepared, assembled and/or modified for a specific process, non-limiting examples of which include immobilization on a solid phase (e.g., a solid support, a flow cell, a bead), enrichment, amplification, cloning, detection and/or for nucleic acid sequencing. In certain embodiments, a nucleic acid library is prepared prior to or during a sequencing process. A nucleic acid library (e.g., sequencing library) can be prepared by a suitable method as known in the art. A nucleic acid library can be prepared by a targeted or a non-targeted preparation process.

In some embodiments, a library of nucleic acids is modified to comprise a chemical moiety (e.g., a functional group) configured for immobilization of nucleic acids to a solid support. In certain embodiments, a library of nucleic acids is modified to comprise a biomolecule (e.g., a functional group) and/or member of a binding pair configured for immobilization of the library to a solid support, non-limiting examples of which include thyroxin-binding globulin, steroid-binding proteins, antibodies, antigens, haptens, enzymes, lectins, nucleic acids, repressors, protein A, protein G, avidin, streptavidin, biotin, complement component Clq, nucleic acid-binding proteins, receptors, carbohydrates, oligonucleotides, polynucleotides, complementary nucleic acid sequences, the like and combinations thereof. Some examples of specific binding pairs include, without limitation: an avidin moiety and a biotin moiety; an antigenic epitope and an antibody or immunologically reactive fragment thereof; an antibody and a hapten; a digoxigen moiety and an anti-digoxigen antibody; a fluorescein moiety and an anti-fluorescein antibody; an operator and a repressor; a nuclease and a nucleotide; a lectin and a polysaccharide; a steroid and a steroid-binding protein; an active compound and an active compound receptor; a hormone and a hormone receptor; an enzyme and a substrate; an immunoglobulin and protein A; an oligonucleotide or polynucleotide and its corresponding complement; the like or combinations thereof.

In some embodiments, a library of nucleic acids is modified to comprise one or more polynucleotides of known composition, non-limiting examples of which include an identifier (e.g., a tag, an indexing tag), a capture sequence, a label, an adapter, a restriction enzyme site, a promoter, an enhancer, an origin of replication, a stem loop, a complimentary sequence (e.g., a primer binding site, an annealing site), a suitable integration site (e.g., a transposon, a viral integration site), a modified nucleotide, the like or combinations thereof. Polynucleotides of known sequence can be added at a suitable position, for example on the 5′ end, 3′ end or within a nucleic acid sequence. Polynucleotides of known sequence can be the same or different sequences. In some embodiments, a polynucleotide of known sequence is configured to hybridize to one or more oligonucleotides immobilized on a surface (e.g., a surface in flow cell). For example, a nucleic acid molecule comprising a 5′ known sequence may hybridize to a first plurality of oligonucleotides while the 3′ known sequence may hybridize to a second plurality of oligonucleotides. In some embodiments, a library of nucleic acid can comprise chromosome-specific tags, capture sequences, labels and/or adapters. In some embodiments, a library of nucleic acids comprises one or more detectable labels. In some embodiments, one or more detectable labels may be incorporated into a nucleic acid library at a 5′ end, at a 3′ end, and/or at any nucleotide position within a nucleic acid in the library. In some embodiments, a library of nucleic acids comprises hybridized oligonucleotides. In certain embodiments, hybridized oligonucleotides are labeled probes. In some embodiments, a library of nucleic acids comprises hybridized oligonucleotide probes prior to immobilization on a solid phase.

In certain embodiments, a ligation-based library preparation method is used (e.g., ILLUMINA TRUSEQ, Illumina, San Diego Calif.). Ligation-based library preparation methods often make use of an adapter (e.g., a methylated adapter) design which can incorporate an index sequence (e.g., a sample index sequence to identify sample origin for a nucleic acid sequence) at the initial ligation step and often can be used to prepare samples for single-read sequencing, paired-end sequencing and multiplexed sequencing. For example, nucleic acids (e.g., fragmented nucleic acids or cell-free DNA) may be end repaired by a fill-in reaction, an exonuclease reaction or a combination thereof. In some embodiments, the resulting blunt-end repaired nucleic acid can then be extended by a single nucleotide, which is complementary to a single nucleotide overhang on the 3′ end of an adapter/primer. Any nucleotide can be used for the extension/overhang nucleotides.

In some embodiments, nucleic acid library preparation comprises ligating an adapter oligonucleotide (e.g., to a sample nucleic acid, to a sample nucleic acid fragment, to a template nucleic acid). Adapter oligonucleotides are often complementary to flow-cell anchors, and sometimes are utilized to immobilize a nucleic acid library to a solid support, such as the inside surface of a flow cell, for example. In some embodiments, an adapter oligonucleotide comprises an identifier, one or more sequencing primer hybridization sites (e.g., sequences complementary to universal sequencing primers, single end sequencing primers, paired end sequencing primers, multiplexed sequencing primers, and the like), or combinations thereof (e.g., adapter/sequencing, adapter/identifier, adapter/identifier/sequencing). In some embodiments, an adapter oligonucleotide comprises one or more of primer annealing polynucleotide (e.g., for annealing to flow cell attached oligonucleotides and/or to free amplification primers), an index polynucleotide (e.g., sample index sequence for tracking nucleic acid from different samples; also referred to as a sample ID), and a barcode polynucleotide (e.g., single molecule barcode (SMB) for tracking individual molecules of sample nucleic acid that are amplified prior to sequencing; also referred to as a molecular barcode). In some embodiments, a primer annealing component of an adapter oligonucleotide comprises one or more universal sequences (e.g., sequences complementary to one or more universal amplification primers). In some embodiments, an index polynucleotide (e.g., sample index; sample ID) is a component of an adapter oligonucleotide. In some embodiments, an index polynucleotide (e.g., sample index; sample ID) is a component of a universal amplification primer sequence.

In some embodiments, adapter oligonucleotides when used in combination with amplification primers (e.g., universal amplification primers) are designed to generate library constructs comprising one or more of: universal sequences, molecular barcodes, sample ID sequences, spacer sequences, and a sample nucleic acid sequence. In some embodiments, adapter oligonucleotides when used in combination with universal amplification primers are designed generate library constructs comprising an ordered combination of one or more of: universal sequences, molecular barcodes, sample ID sequences, spacer sequences, and a sample nucleic acid sequence. For example, a library construct may comprise a first universal sequence, followed by a second universal sequence, followed by first molecular barcode, followed by a spacer sequence, followed by a template sequence (e.g., sample nucleic acid sequence), followed by a spacer sequence, followed by a second molecular barcode, followed by a third universal sequence, followed by a sample ID, followed by a fourth universal sequence. In some embodiments, adapter oligonucleotides when used in combination with amplification primers (e.g., universal amplification primers) are designed generate library constructs for each strand of a template molecule (e.g., sample nucleic acid molecule). In some embodiments, adapter oligonucleotides are duplex adapter oligonucleotides.

A universal sequence is a specific nucleotide sequence that is integrated into two or more nucleic acid molecules or two or more subsets of nucleic acid molecules where the universal sequence is the same for all molecules or subsets of molecules that it is integrated into. A universal sequence is often designed to hybridize to and/or amplify a plurality of different sequences using a single universal primer that is complementary to a universal sequence. In some embodiments, two (e.g., a pair) or more universal sequences and/or universal primers are used. A universal primer often comprises a universal sequence. In some embodiments, adapters (e.g., universal adapters) comprise universal sequences. In some embodiments, one or more universal sequences are used to capture, identify and/or detect multiple species or subsets of nucleic acids.

An identifier can be a suitable detectable label incorporated into or attached to a nucleic acid (e.g., a polynucleotide) that allows detection and/or identification of nucleic acids that comprise the identifier. In some embodiments, an identifier is incorporated into or attached to a nucleic acid during a sequencing method (e.g., by a polymerase). Non-limiting examples of identifiers include nucleic acid tags, nucleic acid indexes or barcodes, a radiolabel (e.g., an isotope), metallic label, a fluorescent label, a chemiluminescent label, a phosphorescent label, a fluorophore quencher, a dye, a protein (e.g., an enzyme, an antibody or part thereof, a linker, a member of a binding pair), the like or combinations thereof. In some embodiments, an identifier (e.g., a nucleic acid index or barcode) is a unique, known and/or identifiable sequence of nucleotides or nucleotide analogues. In some embodiments, identifiers are six or more contiguous nucleotides. A multitude of fluorophores are available with a variety of different excitation and emission spectra. Any suitable type and/or number of fluorophores can be used as an identifier. In some embodiments, 1 or more, 2 or more, 3 or more, 4 or more, 5 or more, 6 or more, 7 or more, 8 or more, 9 or more, 10 or more, 20 or more, 30 or more or 50 or more different identifiers are utilized in a method described herein (e.g., a nucleic acid detection and/or sequencing method). In some embodiments, one or two types of identifiers (e.g., fluorescent labels) are linked to each nucleic acid in a library. Detection and/or quantification of an identifier can be performed by a suitable method, apparatus or machine, non-limiting examples of which include flow cytometry, quantitative polymerase chain reaction (qPCR), gel electrophoresis, a luminometer, a fluorometer, a spectrophotometer, a suitable genechip or microarray analysis, Western blot, mass spectrometry, chromatography, cytofluorimetric analysis, fluorescence microscopy, a suitable fluorescence or digital imaging method, confocal laser scanning microscopy, laser scanning cytometry, affinity chromatography, manual batch mode separation, electric field suspension, a suitable nucleic acid sequencing method and/or nucleic acid sequencing apparatus, the like and combinations thereof.

In other embodiments, a transposon-based library preparation method is used (e.g., EPICENTRE NEXTERA, Epicentre, Madison, Wis.). Transposon-based methods typically use in vitro transposition to simultaneously fragment and tag DNA in a single-tube reaction (often allowing incorporation of platform-specific tags and optional barcodes), and prepare sequencer-ready libraries.

In some embodiments, the nucleic acid library or parts thereof are amplified (e.g., amplified by a PCR-based method). For example, a sequencing method may comprise amplification of a nucleic acid library. A nucleic acid library can be amplified prior to or after immobilization on a solid support (e.g., a solid support in a flow cell). Nucleic acid amplification includes the process of amplifying or increasing the numbers of a nucleic acid template and/or of a complement thereof that are present (e.g., in a nucleic acid library), by producing one or more copies of the template and/or its complement. Amplification can be carried out by a suitable method. A nucleic acid library can be amplified by a thermocycling method or by an isothermal amplification method. In some embodiments, a rolling circle amplification method is used. In some embodiments, amplification takes place on a solid support (e.g., within a flow cell) where a nucleic acid library or portion thereof is immobilized. In certain sequencing methods, a nucleic acid library is added to a flow cell and immobilized by hybridization to anchors under suitable conditions. This type of nucleic acid amplification is often referred to as solid phase amplification. In some embodiments of solid phase amplification, all or a portion of the amplified products are synthesized by an extension initiating from an immobilized primer. Solid phase amplification reactions are analogous to standard solution phase amplifications except that at least one of the amplification oligonucleotides (e.g., primers) is immobilized on a solid support. In some embodiments, modified nucleic acid (e.g., nucleic acid modified by addition of adapters) is amplified.

In some embodiments, solid phase amplification comprises a nucleic acid amplification reaction comprising only one species of oligonucleotide primer immobilized to a surface. In certain embodiments, solid phase amplification comprises a plurality of different immobilized oligonucleotide primer species. In some embodiments, solid phase amplification may comprise a nucleic acid amplification reaction comprising one species of oligonucleotide primer immobilized on a solid surface and a second different oligonucleotide primer species in solution. Multiple different species of immobilized or solution-based primers can be used. Non-limiting examples of solid phase nucleic acid amplification reactions include interfacial amplification, bridge amplification, emulsion PCR, WildFire amplification (e.g., U.S. Patent Application Publication No. 2013/0012399), the like or combinations thereof.

At block 115, the nucleic acid (e.g., nucleic acid fragments, sample nucleic acid, cell-free nucleic acid) from the nucleic acid library is sequenced using a sequencing subsystem. In certain instances, a full or substantially full sequence is obtained and sometimes a partial sequence is obtained. Nucleic acid sequencing generally produces a collection of sequence reads. As used herein, “reads” (e.g., “a read,” “a sequence read”) are short nucleotide sequences produced by any sequencing process described herein or known in the art. Reads can be generated from one end of nucleic acid fragments (“single-end reads”), and sometimes are generated from both ends of nucleic acid fragments (e.g., paired-end reads, double-end reads).

The length of a sequence read is often associated with the particular sequencing technology. High-throughput methods, for example, provide sequence reads that can vary in size from tens to hundreds of base pairs (bp). Nanopore sequencing, for example, can provide sequence reads that can vary in size from tens to hundreds to thousands of base pairs. In some embodiments, sequence reads are of a mean, median, average or absolute length of about 15 bp to about 900 bp long. In certain embodiments, sequence reads are of a mean, median, average or absolute length of about 1000 bp or more. In some embodiments, sequence reads are of a mean, median, average or absolute length of about 1500, 2000, 2500, 3000, 3500, 4000, 4500, or 5000 bp or more. In some embodiments, sequence reads are of a mean, median, average or absolute length of about 100 bp to about 200 bp. In some embodiments, sequence reads are of a mean, median, average or absolute length of about 140 bp to about 160 bp. For example, sequence reads may be of a mean, median, average or absolute length of about 140, 141, 142, 143, 144, 145, 146, 147, 148, 149, 150, 151, 152, 153, 154, 155, 156, 157, 158, 159 or 160 bp.

In some embodiments, the nominal, average, mean or absolute length of single-end reads sometimes is about 10 continuous nucleotides to about 250 or more contiguous nucleotides, about 15 contiguous nucleotides to about 200 or more contiguous nucleotides, about 15 contiguous nucleotides to about 150 or more contiguous nucleotides, about 15 contiguous nucleotides to about 125 or more contiguous nucleotides, about 15 contiguous nucleotides to about 100 or more contiguous nucleotides, about 15 contiguous nucleotides to about 75 or more contiguous nucleotides, about 15 contiguous nucleotides to about 60 or more contiguous nucleotides, 15 contiguous nucleotides to about 50 or more contiguous nucleotides, about 15 contiguous nucleotides to about 40 or more contiguous nucleotides, and sometimes about 15 contiguous nucleotides or about 36 or more contiguous nucleotides. In certain embodiments, the nominal, average, mean or absolute length of single-end reads is about 20 to about 30 bases, or about 24 to about 28 bases in length. In certain embodiments, the nominal, average, mean or absolute length of single-end reads is about 1, 2, 3, 4, 5, 6, 7, 8, 9, 10, 11, 12, 13, 14, 15, 16, 17, 18, 19, 21, 22, 23, 24, 25, 26, 27, 28 or about 29 bases or more in length. In certain embodiments, the nominal, average, mean or absolute length of single-end reads is about 20 to about 200 bases, about 100 to about 200 bases, or about 140 to about 160 bases in length. In certain embodiments, the nominal, average, mean or absolute length of single-end reads is about 30, 40, 50, 60, 70, 80, 90, 100, 110, 120, 130, 140, 150, 160, 170, 180, 190, or about 200 bases or more in length. In certain embodiments, the nominal, average, mean or absolute length of paired-end reads sometimes is about 10 contiguous nucleotides to about 25 contiguous nucleotides or more (e.g., about 10, 11, 12, 13, 14, 15, 16, 17, 18, 19, 20, 21, 22, 23, 24 or 25 nucleotides in length or more), about 15 contiguous nucleotides to about 20 contiguous nucleotides or more, and sometimes is about 17 contiguous nucleotides or about 18 contiguous nucleotides. In certain embodiments, the nominal, average, mean or absolute length of paired-end reads sometimes is about 25 contiguous nucleotides to about 400 contiguous nucleotides or more (e.g., about 25, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, or 400 nucleotides in length or more), about 50 contiguous nucleotides to about 350 contiguous nucleotides or more, about 100 contiguous nucleotides to about 325 contiguous nucleotides, about 150 contiguous nucleotides to about 325 contiguous nucleotides, about 200 contiguous nucleotides to about 325 contiguous nucleotides, about 275 contiguous nucleotides to about 310 contiguous nucleotides, about 100 contiguous nucleotides to about 200 contiguous nucleotides, about 100 contiguous nucleotides to about 175 contiguous nucleotides, about 125 contiguous nucleotides to about 175 contiguous nucleotides, and sometimes is about 140 contiguous nucleotides to about 160 contiguous nucleotides. In certain embodiments, the nominal, average, mean, or absolute length of paired-end reads is about 150 contiguous nucleotides, and sometimes is 150 contiguous nucleotides.

In some embodiments, nucleotide sequence reads obtained from a sample are partial nucleotide sequence reads. As used herein, “partial nucleotide sequence reads” refers to sequence reads of any length with incomplete sequence information, also referred to as sequence ambiguity. Partial nucleotide sequence reads may lack information regarding nucleobase identity and/or nucleobase position or order. Partial nucleotide sequence reads generally do not include sequence reads in which the only incomplete sequence information (or in which less than all of the bases are sequenced or determined) is from inadvertent or unintentional sequencing errors. Such sequencing errors can be inherent to certain sequencing processes and include, for example, incorrect calls for nucleobase identity, and missing or extra nucleobases. Thus, for partial nucleotide sequence reads herein, certain information about the sequence is often deliberately excluded. That is, one deliberately obtains sequence information with respect to less than all of the nucleobases or which might otherwise be characterized as or be a sequencing error. In some embodiments, a partial nucleotide sequence read can span a portion of a nucleic acid fragment. In some embodiments, a partial nucleotide sequence read can span the entire length of a nucleic acid fragment. Partial nucleotide sequence reads are described, for example, in International Patent Application Publication No. WO 2013/052907, the entire content of which is incorporated herein by reference for all purposes.

Reads generally are representations of nucleotide sequences in a physical nucleic acid. For example, in a read containing an ATGC depiction of a sequence, “A” represents an adenine nucleotide, “T” represents a thymine nucleotide, “G” represents a guanine nucleotide and “C” represents a cytosine nucleotide, in a physical nucleic acid. Sequence reads obtained from a sample from a subject can be reads from a mixture of a minority nucleic acid and a majority nucleic acid. For example, sequence reads obtained from the blood of a cancer patient can be reads from a mixture of cancer nucleic acid and non-cancer nucleic acid. In another example, sequence reads obtained from the blood of a pregnant female can be reads from a mixture of fetal nucleic acid and maternal nucleic acid. A mixture of relatively short reads can be transformed by processes described herein into a representation of genomic nucleic acid present in the subject, and/or a representation of genomic nucleic acid present in a tumor or a fetus. In certain instances, a mixture of relatively short reads can be transformed into a representation of a copy number alteration, a genetic variation/genetic alteration or an aneuploidy, for example. In one example, reads of a mixture of cancer and non-cancer nucleic acid can be transformed into a representation of a composite chromosome or a part thereof comprising features of one or both cancer cell and non-cancer cell chromosomes. In another example, reads of a mixture of maternal and fetal nucleic acid can be transformed into a representation of a composite chromosome or a part thereof comprising features of one or both maternal and fetal chromosomes.

In some instances, circulating cell free nucleic acid fragments (CCF fragments) obtained from a cancer patient comprise nucleic acid fragments originating from normal cells (i.e., non-cancer fragments) and nucleic acid fragments originating from cancer cells (i.e., cancer fragments). Sequence reads derived from CCF fragments originating from normal cells (i.e., non-cancerous cells) are referred to herein as “non-cancer reads.” Sequence reads derived from CCF fragments originating from cancer cells are referred to herein as “cancer reads.” CCF fragments from which non-cancer reads are obtained may be referred to herein as non-cancer templates and CCF fragments from which cancer reads are obtained may be referred herein to as cancer templates.

In some instances, circulating cell free nucleic acid fragments (CCF fragments) obtained from a pregnant female comprise nucleic acid fragments originating from fetal cells (i.e., fetal fragments) and nucleic acid fragments originating from maternal cells (i.e., maternal fragments). Sequence reads derived from CCF fragments originating from a fetus are referred to herein as “fetal reads.” Sequence reads derived from CCF fragments originating from the genome of a pregnant female (e.g., a mother) bearing a fetus are referred to herein as “maternal reads.” CCF fragments from which fetal reads are obtained are referred to herein as fetal templates and CCF fragments from which maternal reads are obtained are referred herein to as maternal templates.

In certain embodiments, “obtaining” nucleic acid sequence reads of a sample from a subject and/or “obtaining” nucleic acid sequence reads of a biological specimen from one or more reference persons can involve directly sequencing nucleic acid to obtain the sequence information. In some embodiments, “obtaining” can involve receiving sequence information obtained directly from a nucleic acid by another.

In some embodiments, some or all nucleic acids in a sample are enriched and/or amplified (e.g., non-specifically, e.g., by a PCR based method) prior to or during sequencing. In certain embodiments specific nucleic acid species or subsets in a sample are enriched and/or amplified prior to or during sequencing. In some embodiments, a species or subset of a pre-selected pool of nucleic acids is sequenced randomly. In some embodiments, nucleic acids in a sample are not enriched and/or amplified prior to or during sequencing.

In some embodiments, a representative fraction of a genome is sequenced and is sometimes referred to as “coverage” or “fold coverage.” For example, a 1-fold coverage indicates that roughly 100% of the nucleotide sequences of the genome are represented by reads. In some instances, fold coverage is referred to as (and is directly proportional to) “sequencing depth.” In some embodiments, “fold coverage” is a relative term referring to a prior sequencing run as a reference. For example, a second sequencing run may have 2-fold less coverage than a first sequencing run. In some embodiments, a genome is sequenced with redundancy, where a given region of the genome can be covered by two or more reads or overlapping reads (e.g., a “fold coverage” greater than 1, e.g., a 2-fold coverage). In some embodiments, a genome or a majority of a genome (e.g., genome-wide sequencing) is sequenced with about 0.1-fold to about 100-fold coverage, about 0.2-fold to 20-fold coverage, or about 0.2-fold to about 1-fold coverage (e.g., about 0.2-, 0.3-, 0.4-, 0.5-, 0.6-, 0.7-, 0.8-, 0.9-, 1-, 2-, 3-, 4-, 5-, 6-, 7-, 8-, 9-, 10-, 15-, 20-, 30-, 40-, 50-, 60-, 70-, 80-, 90-fold coverage). In some embodiments, a genome or a majority of a genome (e.g., genome-wide sequencing) is sequenced with about 0.01-fold to about 100-fold coverage, about 0.1-fold to 20-fold coverage, or about 0.1-fold to about 1-fold coverage (e.g., about 0.015-, 0.02-, 0.03-, 0.04-, 0.05-, 0.06-, 0.07-, 0.08-, 0.09-, 0.1-, 0.2-, 0.3-, 0.4-, 0.5-, 0.6-, 0.7-, 0.8-, 0.9-, 1-, 2-, 3-, 4-, 5-, 6-, 7-, 8-, 9-, 10-, 15-, 20-, 30-, 40-, 50-, 60-, 70-, 80-, 90-fold or greater coverage). In some embodiments, specific parts of a genome (e.g., genomic parts from targeted and/or probe-based methods) are sequenced and fold coverage values generally refer to the fraction of the specific genomic parts sequenced (i.e., fold coverage values do not refer to the whole genome). In some instances, specific genomic parts are sequenced at 1000-fold coverage or more. For example, specific genomic parts may be sequenced at 2000-fold, 5,000-fold, 10,000-fold, 20,000-fold, 30,000-fold, 40,000-fold or 50,000-fold coverage. In some embodiments, sequencing is at about 1,000-fold to about 100,000-fold coverage. In some embodiments, sequencing is at about 10,000-fold to about 70,000-fold coverage. In some embodiments, sequencing is at about 20,000-fold to about 60,000-fold coverage. In some embodiments, sequencing is at about 30,000-fold to about 50,000-fold coverage.

In some embodiments, one nucleic acid sample from one individual is sequenced. In certain embodiments, nucleic acids from each of two or more samples are sequenced, where samples are from one individual or from different individuals. In certain embodiments, nucleic acid samples from two or more biological samples are pooled, where each biological sample is from one individual or two or more individuals, and the pool is sequenced. In the latter embodiments, a nucleic acid sample from each biological sample often is identified by one or more unique identifiers.

In some embodiments, a sequencing method utilizes identifiers that allow multiplexing of sequence reactions in a sequencing process. The greater the number of unique identifiers, the greater the number of samples and/or chromosomes for detection, for example, that can be multiplexed in a sequencing process. A sequencing process can be performed using any suitable number of unique identifiers (e.g., 4, 8, 12, 24, 48, 96, 1000, 10000, 108, 1024, or more).

A sequencing process sometimes makes use of a solid phase, and sometimes the solid phase comprises a flow cell on which nucleic acid from a library can be attached and reagents can be flowed and contacted with the attached nucleic acid. A flow cell sometimes includes flow cell lanes, and use of identifiers can facilitate analyzing a number of samples in each lane. A flow cell often is a solid support that can be configured to retain and/or allow the orderly passage of reagent solutions over bound analytes. Flow cells frequently are planar in shape, optically transparent, generally in the millimeter or sub-millimeter scale, and often have channels or lanes in which the analyte/reagent interaction occurs. In some embodiments, the number of samples analyzed in a given flow cell lane is dependent on the number of unique identifiers utilized during library preparation and/or probe design. Multiplexing using 12 identifiers, for example, allows simultaneous analysis of 96 samples (e.g., equal to the number of wells in a 96-well microwell plate) in an 8-lane flow cell. Similarly, multiplexing using 48 identifiers, for example, allows simultaneous analysis of 384 samples (e.g., equal to the number of wells in a 384-well microwell plate) in an 8-lane flow cell. Non-limiting examples of commercially available multiplex sequencing kits include Illumina's multiplexing sample preparation oligonucleotide kit and multiplexing sequencing primers and PhiX control kit (e.g., Illumina's catalog numbers PE-400-1001 and PE-400-1002, respectively).

Any suitable method of sequencing nucleic acids can be used, non-limiting examples of which include Maxim & Gilbert, chain-termination methods, sequencing by synthesis, sequencing by ligation, sequencing by mass spectrometry, microscopy-based techniques, the like or combinations thereof. In some embodiments, a first generation technology, such as, for example, Sanger sequencing methods including automated Sanger sequencing methods, including microfluidic Sanger sequencing, can be used in a method provided herein. In some embodiments, sequencing technologies that include the use of nucleic acid imaging technologies (e.g., transmission electron microscopy (TEM) and atomic force microscopy (AFM)), can be used. In some embodiments, a high-throughput sequencing method is used. High-throughput sequencing methods generally involve clonally amplified DNA templates or single DNA molecules that are sequenced in a massively parallel fashion, sometimes within a flow cell. Next generation (e.g., 2nd and 3rd generation) sequencing techniques capable of sequencing DNA in a massively parallel fashion can be used for methods described herein and are collectively referred to herein as “massively parallel sequencing” (MPS). In some embodiments, MPS sequencing methods utilize a targeted approach, where specific chromosomes, genes or regions of interest are sequenced. In certain embodiments, a non-targeted approach is used where most or all nucleic acids in a sample are sequenced, amplified and/or captured randomly.

In some embodiments, a targeted enrichment, amplification and/or sequencing approach is used. A targeted approach often isolates, selects and/or enriches a subset of nucleic acids in a sample for further processing by use of sequence-specific oligonucleotides. In some embodiments, a library of sequence-specific oligonucleotides is utilized to target (e.g., hybridize to) one or more sets of nucleic acids in a sample. Sequence-specific oligonucleotides and/or primers are often selective for particular sequences (e.g., unique nucleic acid sequences) present in one or more chromosomes, genes, exons, introns, and/or regulatory regions of interest. Any suitable method or combination of methods can be used for enrichment, amplification and/or sequencing of one or more subsets of targeted nucleic acids. In some embodiments, targeted sequences are isolated and/or enriched by capture to a solid phase (e.g., a flow cell, a bead) using one or more sequence-specific anchors. In some embodiments, targeted sequences are enriched and/or amplified by a polymerase-based method (e.g., a PCR-based method, by any suitable polymerase-based extension) using sequence-specific primers and/or primer sets. Sequence specific anchors often can be used as sequence-specific primers.

MPS sequencing sometimes makes use of sequencing by synthesis and certain imaging processes. A nucleic acid sequencing technology that may be used in a method described herein is sequencing-by-synthesis and reversible terminator-based sequencing (e.g., Illumina's Genome Analyzer; Genome Analyzer II; HISEQ 2000; HISEQ 2500 (Illumina, San Diego Calif.)). With this technology, millions of nucleic acid (e.g., DNA) fragments can be sequenced in parallel. In one example of this type of sequencing technology, a flow cell is used which contains an optically transparent slide with 8 individual lanes on the surfaces of which are bound oligonucleotide anchors (e.g., adapter primers).

Sequencing by synthesis generally is performed by iteratively adding (e.g., by covalent addition) a nucleotide to a primer or preexisting nucleic acid strand in a template directed manner. Each iterative addition of a nucleotide is detected and the process is repeated multiple times until a sequence of a nucleic acid strand is obtained. The length of a sequence obtained depends, in part, on the number of addition and detection steps that are performed. In some embodiments of sequencing by synthesis, one, two, three or more nucleotides of the same type (e.g., A, G, C or T) are added and detected in a round of nucleotide addition. Nucleotides can be added by any suitable method (e.g., enzymatically or chemically). For example, in some embodiments, a polymerase or a ligase adds a nucleotide to a primer or to a preexisting nucleic acid strand in a template directed manner. In some embodiments of sequencing by synthesis, different types of nucleotides, nucleotide analogues and/or identifiers are used. In some embodiments, reversible terminators and/or removable (e.g., cleavable) identifiers are used. In some embodiments, fluorescent labeled nucleotides and/or nucleotide analogues are used. In certain embodiments, sequencing by synthesis comprises a cleavage (e.g., cleavage and removal of an identifier) and/or a washing step. In some embodiments, the addition of one or more nucleotides is detected by a suitable method described herein or known in the art, non-limiting examples of which include any suitable imaging apparatus, a suitable camera, a digital camera, a CCD (Charge Couple Device) based imaging apparatus (e.g., a CCD camera), a CMOS (Complementary Metal Oxide Silicon) based imaging apparatus (e.g., a CMOS camera), a photo diode (e.g., a photomultiplier tube), electron microscopy, a field-effect transistor (e.g., a DNA field-effect transistor), an ISFET ion sensor (e.g., a CHEMFET sensor), the like or combinations thereof.

Any suitable MPS method, system or technology platform for conducting methods described herein can be used to obtain nucleic acid sequence reads. Non-limiting examples of MPS platforms include Illumina/Solex/HiSeq (e.g., Illumina's Genome Analyzer; Genome Analyzer II; HISEQ 2000; HISEQ), SOLID, Roche/454, PACBIO and/or SMRT, Helicos True Single Molecule Sequencing, Ion Torrent and Ion semiconductor-based sequencing (e.g., as developed by Life Technologies), WildFire, 5500, 5500xl W and/or 5500xl W Genetic Analyzer based technologies (e.g., as developed and sold by Life Technologies, U.S. Patent Application Publication No. 2013/0012399); Polony sequencing, Pyrosequencing, Massively Parallel Signature Sequencing (MPSS), RNA polymerase (RNAP) sequencing, LaserGen systems and methods, Nanopore-based platforms, chemical-sensitive field effect transistor (CHEMFET) array, electron microscopy-based sequencing (e.g., as developed by ZS Genetics, Halcyon Molecular), nanoball sequencing, the like or combinations thereof. Other sequencing methods that may be used to conduct methods herein include digital PCR, sequencing by hybridization, nanopore sequencing, chromosome-specific sequencing (e.g., using DANSR (digital analysis of selected regions) technology.

In some embodiments, sequence reads are generated, obtained, gathered, assembled, manipulated, transformed, processed, and/or provided by a sequence subsystem. A machine comprising a sequence subsystem can be a suitable machine and/or apparatus that determines the sequence of a nucleic acid utilizing a sequencing technology known in the art. In some embodiments, a sequence subsystem can align, assemble, fragment, complement, reverse complement, and/or error check (e.g., error correct sequence reads).

At block 120, the sequence reads are processed using a sequence processing subsystem to obtain sequence read data. The processing of the sequence reads includes read alignment, mapping, and filtering.

### Alignment and Mapping

In some embodiments, one or more processing steps comprises aligning and mapping sequence reads. Sequence reads can be mapped and a number of reads mapping to a specified nucleic acid region (e.g., a chromosome or portion thereof) are referred to as counts. Any suitable mapping method (e.g., process, algorithm, program, software, subsystem, the like or combination thereof) can be used. Certain aspects of mapping processes are described hereafter. Mapping nucleotide sequence reads (i.e., sequence information from a fragment whose physical genomic position is unknown) can be performed in a number of ways, and often comprises alignment of the obtained sequence reads with a matching sequence in a reference genome. In such alignments, sequence reads generally are aligned to a reference sequence and those that align are designated as being “mapped,” as “a mapped sequence read” or as “a mapped read.” In certain embodiments, a mapped sequence read is referred to as a “hit” or “count.” In some embodiments, mapped sequence reads are grouped together according to various parameters and assigned to particular genomic portions, which are discussed in further detail below.

The terms “aligned,” “alignment,” or “aligning” generally refer to two or more nucleic acid sequences that can be identified as a match (e.g., 100% identity) or partial match. Alignments can be done manually or by a computer (e.g., a software, program, subsystem, or algorithm), non-limiting examples of which include the Efficient Local Alignment of Nucleotide Data (ELAND) computer program distributed as part of the Illumina Genomics Analysis pipeline. Alignment of a sequence read can be a 100% sequence match. In some cases, an alignment is less than a 100% sequence match (i.e., non-perfect match, partial match, partial alignment). In some embodiments, an alignment is about a 99%, 98%, 97%, 96%, 95%, 94%, 93%, 92%, 91%, 90%, 89%, 88%, 87%, 86%, 85%, 84%, 83%, 82%, 81%, 80%, 79%, 78%, 77%, 76% or 75% match. In some embodiments, an alignment comprises a mismatch. In some embodiments, an alignment comprises 1, 2, 3, 4 or 5 mismatches. Two or more sequences can be aligned using either strand (e.g., sense or antisense strand). In certain embodiments, a nucleic acid sequence is aligned with the reverse complement of another nucleic acid sequence.

Various computational methods can be used to map each sequence read to a portion. Non-limiting examples of computer algorithms that can be used to align sequences include, without limitation, BLAST, BLITZ, FASTA, BOWTIE 1, BOWTIE 2, ELAND, MAQ, PROBEMATCH, SOAP, BWA or SEQMAP, or variations thereof or combinations thereof. In some embodiments, sequence reads can be aligned with sequences in a reference genome. In some embodiments, sequence reads can be found and/or aligned with sequences in nucleic acid databases known in the art including, for example, GenBank, dbEST, dbSTS, EMBL (European Molecular Biology Laboratory) and DDBJ (DNA Databank of Japan). BLAST or similar tools can be used to search identified sequences against a sequence database. Search hits can then be used to sort the identified sequences into appropriate portions (described hereafter), for example.

In some embodiments, a read may uniquely or non-uniquely map to portions in a reference genome. A read is considered as “uniquely mapped” if it aligns with a single sequence in the reference genome. A read is considered as “non-uniquely mapped” if it aligns with two or more sequences in the reference genome. In some embodiments, non-uniquely mapped reads are eliminated from further analysis (e.g. quantification). A certain, small degree of mismatch (0-1) may be allowed to account for single nucleotide polymorphisms that may exist between the reference genome and the reads from individual samples being mapped, in certain embodiments. In some embodiments, no degree of mismatch is allowed for a read mapped to a reference sequence.

As used herein, the term “reference genome” can refer to any particular known, sequenced or characterized genome, whether partial or complete, of any organism or virus which may be used to reference identified sequences from a subject. For example, a reference genome used for human subjects as well as many other organisms can be found at the National Center for Biotechnology Information at World Wide Web URL ncbi.nlm.nih.gov. A “genome” refers to the complete genetic information of an organism or virus, expressed in nucleic acid sequences. As used herein, a reference sequence or reference genome often is an assembled or partially assembled genomic sequence from an individual or multiple individuals. In some embodiments, a reference genome is an assembled or partially assembled genomic sequence from one or more human individuals. In some embodiments, a reference genome comprises sequences assigned to chromosomes.

In certain embodiments, mappability is assessed for a genomic region (e.g., portion, genomic portion). Mappability is the ability to unambiguously align a nucleotide sequence read to a portion of a reference genome, typically up to a specified number of mismatches, including, for example, 0, 1, 2 or more mismatches. For a given genomic region, the expected mappability can be estimated using a sliding-window approach of a preset read length and averaging the resulting read-level mappability values. Genomic regions comprising stretches of unique nucleotide sequence sometimes have a high mappability value.

For paired-end sequencing, reads may be mapped to a reference genome by use of a suitable mapping and/or alignment program, non-limiting examples of which include BWA (Li H. and Durbin R. (2009) Bioinformatics 25, 1754-60), Novoalign [Novocraft (2010)], Bowtie (Langmead B, et al., (2009) Genome Biol. 10: R25), SOAP2 (Li R, et al., (2009) Bioinformatics 25, 1966-67), BFAST (Homer N, et al., (2009) PloS ONE 4, e7767), GASSST (Rizk, G. and Lavenier, D. (2010) Bioinformatics 26, 2534-2540), and Mpscan (Rivals E., et al. (2009) Lecture Notes in Computer Science 5724, 246-260), and the like. Paired-end reads may be mapped and/or aligned using a suitable short read alignment program. Non-limiting examples of short read alignment programs include BarraCUDA, BFAST, BLASTN, BLAT, Bowtie, BWA, CASHX, CUDA-EC, CUSHAW, CUSHAW2, drFAST, ELAND, ERNE, GNUMAP, GEM, GensearchNGS, GMAP, Geneious Assembler, ISAAC, LAST, MAQ, mrFAST, mrsFAST, MOSAIK, Mpscan, Novoalign, NovoalignCS, Novocraft, NextGENe, Omixon, PALMapper, Partek, PASS, PerM, Qpalma, RazerS, REAL, cREAL, RMAP, rNA, RTG, Segemehl, SeqMap, Shrec, SHRIMP, SLIDER, SOAP, SOAP2, SOAP3, SOCS, SSAHA, SSAHA2, Stampy, StoRM, Subread, Subjunc, Taipan, UGENE, VelociMapper, TimeLogic, XpressAlign, ZOOM, the like or combinations thereof. Paired-end reads are often mapped to opposing ends of the same polynucleotide fragment, according to a reference genome. In some embodiments, read mates are mapped independently. In some embodiments, information from both sequence reads (i.e., from each end) is factored in the mapping process. A reference genome is often used to determine and/or infer the sequence of nucleic acids located between paired-end read mates. The term “discordant read pairs” as used herein refers to a paired-end read comprising a pair of read mates, where one or both read mates fail to unambiguously map to the same region of a reference genome defined, in part, by a segment of contiguous nucleotides. In some embodiments, discordant read pairs are paired-end read mates that map to unexpected locations of a reference genome. Non-limiting examples of unexpected locations of a reference genome include (i) two different chromosomes, (ii) locations separated by more than a predetermined fragment size (e.g., more than 300 bp, more than 500 bp, more than 1000 bp, more than 5000 bp, or more than 10,000 bp), (iii) an orientation inconsistent with a reference sequence (e.g., opposite orientations), the like or a combination thereof. In some embodiments, discordant read mates are identified according to a length (e.g., an average length, a predetermined fragment size) or expected length of template polynucleotide fragments in a sample. For example, read mates that map to a location that is separated by more than the average length or expected length of polynucleotide fragments in a sample are sometimes identified as discordant read pairs. Read pairs that map in opposite orientation are sometimes determined by taking the reverse complement of one of the reads and comparing the alignment of both reads using the same strand of a reference sequence. Discordant read pairs can be identified by any suitable method and/or algorithm known in the art or described herein (e.g., SVDetect, Lumpy, BreakDancer, BreakDancerMax, CREST, DELLY, the like or combinations thereof).

### Portioning

In some embodiments, mapped sequence reads are grouped together according to various parameters and assigned to particular genomic portions (e.g., portions of a reference genome). A “portion” also may be referred to herein as a “genomic section,” “bin,” “partition,” “portion of a reference genome,” “portion of a chromosome” or “genomic portion.” A portion often is defined by partitioning of a genome according to one or more features. Non-limiting examples of certain partitioning features include length (e.g., fixed length, non-fixed length) and other structural features. Genomic portions sometimes include one or more of the following features: fixed length, non-fixed length, random length, non-random length, equal length, unequal length (e.g., at least two of the genomic portions are of unequal length), do not overlap (e.g., the 3′ ends of the genomic portions sometimes abut the 5′ ends of adjacent genomic portions), overlap (e.g., at least two of the genomic portions overlap), contiguous, consecutive, not contiguous, and not consecutive. Genomic portions sometimes are about 1 to about 1,000 kilobases in length (e.g., about 2, 3, 4, 5, 6, 7, 8, 9, 10, 15, 20, 25, 30, 35, 40, 45, 50, 55, 60, 65, 70, 75, 80, 85, 90, 95, 100, 200, 300, 400, 500, 600, 700, 800, 900 kilobases in length), about 5 to about 500 kilobases in length, about 10 to about 100 kilobases in length, or about 40 to about 60 kilobases in length.

Partitioning sometimes is based on, or is based in part on, certain informational features, such as, information content and information gain, for example. Non-limiting examples of certain informational features include speed and/or convenience of alignment, sequencing coverage variability, GC content (e.g., stratified GC content, particular GC contents, high or low GC content), uniformity of GC content, other measures of sequence content (e.g., fraction of individual nucleotides, fraction of pyrimidines or purines, fraction of natural vs. non-natural nucleic acids, fraction of methylated nucleotides, and CpG content), methylation state, duplex melting temperature, amenability to sequencing or PCR, uncertainty value assigned to individual portions of a reference genome, and/or a targeted search for particular features. In some embodiments, information content may be quantified using a p-value profile measuring the significance of particular genomic locations for distinguishing between groups of confirmed normal and abnormal subjects (e.g. euploid and trisomy subjects, respectively).

In some embodiments, partitioning a genome may eliminate similar regions (e.g., identical or homologous regions or sequences) across a genome and only keep unique regions. Regions removed during partitioning may be within a single chromosome, may be one or more chromosomes, or may span multiple chromosomes. In some embodiments, a partitioned genome is reduced and optimized for faster alignment, often focusing on uniquely identifiable sequences.

In some embodiments, genomic portions result from a partitioning based on non-overlapping fixed size, which results in consecutive, non-overlapping portions of fixed length. Such portions often are shorter than a chromosome and often are shorter than a copy number variation (or copy number alteration) region (e.g., a region that is duplicated or is deleted), the latter of which can be referred to as a segment. A “segment” or “genomic segment” often includes two or more fixed-length genomic portions, and often includes two or more consecutive fixed-length portions (e.g., about 2 to about 100 such portions (e.g., 2, 3, 4, 5, 6, 7, 8, 9, 10, 11, 12, 13, 14, 15, 16, 17, 18, 19, 20, 25, 30, 35, 40, 45, 50, 60, 70, 80, 90 such portions)).

Multiple portions sometimes are analyzed in groups, and sometimes reads mapped to portions are quantified according to a particular group of genomic portions. Where portions are partitioned by structural features and correspond to regions in a genome, portions sometimes are grouped into one or more segments and/or one or more regions. Non-limiting examples of regions include sub-chromosome (i.e., shorter than a chromosome), chromosome, autosome, sex chromosome and combinations thereof. One or more sub-chromosome regions sometimes are genes, gene fragments, regulatory sequences, introns, exons, segments (e.g., a segment spanning a copy number alteration region; a segment spanning a copy number variation region), microduplications, microdeletions and the like. A region sometimes is smaller than a chromosome of interest or is the same size of a chromosome of interest, and sometimes is smaller than a reference chromosome or is the same size as a reference chromosome.

Filtering and/or Selecting Portions

In some embodiments, one or more processing steps comprise one or more portion filtering steps and/or portion selection steps. The term “filtering” as used herein refers to removing portions or portions of a reference genome from consideration. In certain embodiments, one or more portions are filtered (e.g., subjected to a filtering process) thereby providing filtered portions. In some embodiments, a filtering process removes certain portions and retains portions (e.g., a subset of portions). Following a filtering process, retained portions are often referred to herein as filtered portions.

Portions of a reference genome can be selected for removal based on any suitable criteria, including but not limited to redundant data (e.g., redundant or overlapping mapped reads), non-informative data (e.g., portions of a reference genome with zero median counts), portions of a reference genome with over represented or underrepresented sequences, noisy data, the like, or combinations of the foregoing. A filtering process often involves removing one or more portions of a reference genome from consideration and subtracting the counts in the one or more portions of a reference genome selected for removal from the counted or summed counts for the portions of a reference genome, chromosome or chromosomes, or genome under consideration. In some embodiments, portions of a reference genome can be removed successively (e.g., one at a time to allow evaluation of the effect of removal of each individual portion), and in certain embodiments, all portions of a reference genome marked for removal can be removed at the same time. In some embodiments, portions of a reference genome characterized by a variance above or below a certain level are removed, which sometimes is referred to herein as filtering “noisy” portions of a reference genome. In certain embodiments, a filtering process comprises obtaining data points from a data set that deviate from the mean profile level of a portion, a chromosome, or part of a chromosome by a predetermined multiple of the profile variance, and in certain embodiments, a filtering process comprises removing data points from a data set that do not deviate from the mean profile level of a portion, a chromosome or part of a chromosome by a predetermined multiple of the profile variance. In some embodiments, a filtering process is utilized to reduce the number of candidate portions of a reference genome analyzed for the presence or absence of a genetic variation/genetic alteration and/or copy number alteration (e.g., aneuploidy, microdeletion, microduplication). Reducing the number of candidate portions of a reference genome analyzed for the presence or absence of a genetic variation/genetic alteration and/or copy number alteration often reduces the complexity and/or dimensionality of a data set, and sometimes increases the speed of searching for and/or identifying genetic variations/genetic alterations and/or copy number alterations by two or more orders of magnitude.

Portions may be processed (e.g., filtered and/or selected) by any suitable method and according to any suitable parameter. Non-limiting examples of features and/or parameters that can be used to filter and/or select portions include redundant data (e.g., redundant or overlapping mapped reads), non-informative data (e.g., portions of a reference genome with zero mapped counts), portions of a reference genome with over represented or underrepresented sequences, noisy data, counts, count variability, coverage, mappability, variability, a repeatability measure, read density, variability of read density, a level of uncertainty, guanine-cytosine (GC) content, CCF fragment length and/or read length (e.g., a fragment length ratio (FLR), a fetal ratio statistic (FRS)), Dnasel-sensitivity, methylation state, acetylation, histone distribution, chromatin structure, percent repeats, the like or combinations thereof. Portions can be filtered and/or selected according to any suitable feature or parameter that correlates with a feature or parameter listed or described herein. Portions can be filtered and/or selected according to features or parameters that are specific to a portion (e.g., as determined for a single portion according to multiple samples) and/or features or parameters that are specific to a sample (e.g., as determined for multiple portions within a sample). In some embodiments, portions are filtered and/or removed according to relatively low mappability, relatively high variability, a high level of uncertainty, relatively long CCF fragment lengths (e.g., low FRS, low FLR), relatively large fraction of repetitive sequences, high GC content, low GC content, low counts, zero counts, high counts, the like, or combinations thereof. In some embodiments, portions (e.g., a subset of portions) are selected according to suitable level of mappability, variability, level of uncertainty, fraction of repetitive sequences, count, GC content, the like, or combinations thereof. In some embodiments, portions (e.g., a subset of portions) are selected according to relatively short CCF fragment lengths (e.g., high FRS, high FLR). Counts and/or reads mapped to portions are sometimes processed (e.g., normalized) prior to and/or after filtering or selecting portions (e.g., a subset of portions). In some embodiments, counts and/or reads mapped to portions are not processed prior to and/or after filtering or selecting portions (e.g., a subset of portions).

In some embodiments, portions may be filtered according to a measure of error (e.g., standard deviation, standard error, calculated variance, p-value, mean absolute error (MAE), average absolute deviation and/or mean absolute deviation (MAD)). In certain instances, a measure of error may refer to count variability. In some embodiments, portions are filtered according to count variability. In certain embodiments, count variability is a measure of error determined for counts mapped to a portion (i.e., portion) of a reference genome for multiple samples (e.g., multiple samples obtained from multiple subjects, e.g., 50 or more, 100 or more, 500 or more 1000 or more, 5000 or more or 10,000 or more subjects). In some embodiments, portions with a count variability above a pre-determined upper range are filtered (e.g., excluded from consideration). In some embodiments, portions with a count variability below a pre-determined lower range are filtered (e.g., excluded from consideration). In some embodiments, portions with a count variability outside a pre-determined range are filtered (e.g., excluded from consideration). In some embodiments, portions with a count variability within a pre-determined range are selected (e.g., used for determining the presence or absence of a copy number alteration). In some embodiments, count variability of portions represents a distribution (e.g., a normal distribution). In some embodiments, portions are selected within a quantile of the distribution. In some embodiments, portions within a 99% quantile of the distribution of count variability are selected.

Sequence reads from any suitable number of samples can be utilized to identify a subset of portions that meet one or more criteria, parameters and/or features described herein. Sequence reads from a group of samples from multiple subjects sometimes are utilized. In some embodiments, the multiple subjects include pregnant females. In some embodiments, the multiple subjects include healthy subjects. In some embodiments, the multiple subjects include cancer patients. One or more samples from each of the multiple subjects can be addressed (e.g., 1 to about 20 samples from each subject (e.g., about 2, 3, 4, 5, 6, 7, 8, 9, 10, 11, 12, 13, 14, 15, 16, 17, 18 or 19 samples)), and a suitable number of subjects may be addressed (e.g., about 2 to about 10,000 subjects (e.g., about 10, 20, 30, 40, 50, 60, 70, 80, 90, 100, 150, 200, 250, 300, 350, 400, 500, 600, 700, 800, 900, 1000, 2000, 3000, 4000, 5000, 6000, 7000, 8000, 9000 subjects)). In some embodiments, sequence reads from the same test sample(s) from the same subject are mapped to portions in the reference genome and are used to generate the subset of portions.

Portions can be selected and/or filtered by any suitable method. In some embodiments, portions are selected according to visual inspection of data, graphs, plots and/or charts. In certain embodiments, portions are selected and/or filtered (e.g., in part) by a system or a machine comprising one or more microprocessors and memory. In some embodiments, portions are selected and/or filtered (e.g., in part) by a non-transitory computer-readable storage medium with an executable program stored thereon, where the program instructs a microprocessor to perform the selecting and/or filtering.

In some embodiments, sequence reads derived from a sample are mapped to all or most portions of a reference genome and a pre-selected subset of portions are thereafter selected. For example, a subset of portions to which reads from fragments under a particular length threshold preferentially map may be selected. Certain methods for pre-selecting a subset of portions are described in U.S. Patent Application Publication No. 2014/0180594, the entire content of which is incorporated herein by reference for all purposes. Reads from a selected subset of portions often are utilized in further steps of a determination of the presence or absence of a genetic variation or genetic alteration, for example. Often, reads from portions not selected are not utilized in further steps of a determination of the presence or absence of a genetic variation or genetic alteration (e.g., reads in the non-selected portions are removed or filtered).

In some embodiments, portions associated with read densities (e.g., where a read density is for a portion) are removed by a filtering process and read densities associated with removed portions are not included in a determination of the presence or absence of a copy number alteration (e.g., a chromosome aneuploidy, microduplication, microdeletion). In some embodiments, a read density profile comprises and/or consists of read densities of filtered portions. Portions are sometimes filtered according to a distribution of counts and/or a distribution of read densities. In some embodiments, portions are filtered according to a distribution of counts and/or read densities where the counts and/or read densities are obtained from one or more reference samples. One or more reference samples may be referred to herein as a training set. In some embodiments, portions are filtered according to a distribution of counts and/or read densities where the counts and/or read densities are obtained from one or more test samples. In some embodiments, portions are filtered according to a measure of uncertainty for a read density distribution. In certain embodiments, portions that demonstrate a large deviation in read densities are removed by a filtering process. For example, a distribution of read densities (e.g., a distribution of average mean, or median read densities) can be determined, where each read density in the distribution maps to the same portion. A measure of uncertainty (e.g., a MAD) can be determined by comparing a distribution of read densities for multiple samples where each portion of a genome is associated with measure of uncertainty. According to the foregoing example, portions can be filtered according to a measure of uncertainty (e.g., a standard deviation (SD), a MAD) associated with each portion and a predetermined threshold. In certain instances, portions comprising MAD values within the acceptable range are retained and portions comprising MAD values outside of the acceptable range are removed from consideration by a filtering process. In some embodiments, according to the foregoing example, portions comprising read densities values (e.g., median, average, or mean read densities) outside a pre-determined measure of uncertainty are often removed from consideration by a filtering process. In some embodiments, portions comprising read densities values (e.g., median, average, or mean read densities) outside an inter-quartile range of a distribution are removed from consideration by a filtering process. In some embodiments, portions comprising read densities values outside more than 2 times, 3 times, 4 times or 5 times an inter-quartile range of a distribution are removed from consideration by a filtering process. In some embodiments, portions comprising read densities values outside more than 2 sigma, 3 sigma, 4 sigma, 5 sigma, 6 sigma, 7 sigma or 8 sigma (e.g., where sigma is a range defined by a standard deviation) are removed from consideration by a filtering process.

At block 125, the sequence read data is processed using a data processing subsystem to obtain processed sequence read data. The processing of the sequence read data includes quantification, assignment of levels or classes, data normalization, or any combination thereof.

### Sequence Read Quantification

Sequence reads that are mapped or partitioned based on a selected feature or variable can be quantified to determine the amount or number of reads that are mapped to one or more portions (e.g., portion of a reference genome), in some embodiments. In certain embodiments, the quantity of sequence reads that are mapped to a portion or segment is referred to as a count or read density. A count often is associated with a genomic portion. In some embodiments, a count is determined from some or all of the sequence reads mapped to (I.e., associated with) a portion. In certain embodiments, a count is determined from some or all of the sequence reads mapped to a group of portions (e.g., portions in a segment or region (described herein)).

A count can be determined by a suitable method, operation or mathematical process. A count sometimes is the direct sum of all sequence reads mapped to a genomic portion or a group of genomic portions corresponding to a segment, a group of portions corresponding to a sub-region of a genome (e.g., copy number variation region, copy number alteration region, copy number duplication region, copy number deletion region, microduplication region, microdeletion region, chromosome region, autosome region, sex chromosome region or other chromosomal rearrangement) and/or sometimes is a group of portions corresponding to a genome. A read quantification sometimes is a ratio, and sometimes is a ratio of a quantification for portion(s) in region a to a quantification for portion(s) in region b. Region a sometimes is one portion, segment region, copy number variation region, copy number alteration region, copy number duplication region, copy number deletion region, microduplication region, microdeletion region, chromosome region, autosome region and/or sex chromosome region. Region b independently sometimes is one portion, segment region, copy number variation region, copy number alteration region, copy number duplication region, copy number deletion region, microduplication region, microdeletion region, chromosome region, autosome region, sex chromosome region, a region including all autosomes, a region including sex chromosomes and/or a region including all chromosomes.

In some embodiments, a count is derived from raw sequence reads and/or filtered sequence reads. In certain embodiments, a count is determined by a mathematical process. In certain embodiments, a count is an average, mean or sum of sequence reads mapped to a genomic portion or group of genomic portions (e.g., genomic portions in a region). In some embodiments, a count is associated with an uncertainty value. A count sometimes is adjusted. A count may be adjusted according to sequence reads associated with a genomic portion or group of portions that have been weighted, removed, filtered, normalized, adjusted, averaged, derived as a mean, derived as a median, added, or combination thereof.

A sequence read quantification sometimes is a read density. A read density may be determined and/or generated for one or more segments of a genome. In certain instances, a read density may be determined and/or generated for one or more chromosomes. In some embodiments, a read density comprises a quantitative measure of counts of sequence reads mapped to a segment or portion of a reference genome. A read density can be determined by a suitable process. In some embodiments, a read density is determined by a suitable distribution and/or a suitable distribution function. Non-limiting examples of a distribution function include a probability function, probability distribution function, probability density function (PDF), a kernel density function (kernel density estimation), a cumulative distribution function, probability mass function, discrete probability distribution, an absolutely continuous univariate distribution, the like, any suitable distribution, or combinations thereof. A read density may be a density estimation derived from a suitable probability density function. A density estimation is the construction of an estimate, based on observed data, of an underlying probability density function. In some embodiments, a read density comprises a density estimation (e.g., a probability density estimation, a kernel density estimation). A read density may be generated according to a process comprising generating a density estimation for each of the one or more portions of a genome where each portion comprises counts of sequence reads. A read density may be generated for normalized and/or weighted counts mapped to a portion or segment. In some instances, each read mapped to a portion or segment may contribute to a read density, a value (e.g., a count) equal to its weight obtained from a normalization process described herein. In some embodiments, read densities for one or more portions or segments are adjusted. Read densities can be adjusted by a suitable method. For example, read densities for one or more portions can be weighted and/or normalized.

Reads quantified for a given portion or segment can be from one source or different sources. In one example, reads may be obtained from nucleic acid from a subject having cancer or suspected of having cancer. In such circumstances, reads mapped to one or more portions often are reads representative of both healthy cells (i.e., non-cancer cells) and cancer cells (e.g., tumor cells). In certain embodiments, some of the reads mapped to a portion are from cancer cell nucleic acid and some of the reads mapped to the same portion are from non-cancer cell nucleic acid. In another example, reads may be obtained from a nucleic acid sample from a pregnant female bearing a fetus. In such circumstances, reads mapped to one or more portions often are reads representative of both the fetus and the mother of the fetus (e.g., a pregnant female subject). In certain embodiments, some of the reads mapped to a portion are from a fetal genome and some of the reads mapped to the same portion are from a maternal genome.

### Levels

In some embodiments, a value (e.g., a number, a quantitative value) is ascribed to a level. A level can be determined by a suitable method, operation or mathematical process (e.g., a processed level). A level often is, or is derived from, counts (e.g., normalized counts) for a set of portions. In some embodiments, a level of a portion is substantially equal to the total number of counts mapped to a portion (e.g., counts, normalized counts). Often a level is determined from counts that are processed, transformed or manipulated by a suitable method, operation or mathematical process known in the art. In some embodiments, a level is derived from counts that are processed and non-limiting examples of processed counts include weighted, removed, filtered, normalized, adjusted, averaged, derived as a mean (e.g., mean level), added, subtracted, transformed counts or combination thereof. In some embodiments, a level comprises counts that are normalized (e.g., normalized counts of portions). A level can be for counts normalized by a suitable process, non-limiting examples of which are described herein. A level can comprise normalized counts or relative amounts of counts. In some embodiments, a level is for counts or normalized counts of two or more portions that are averaged and the level is referred to as an average level. In some embodiments, a level is for a set of portions having a mean count or mean of normalized counts which is referred to as a mean level. In some embodiments, a level is derived for portions that comprise raw and/or filtered counts. In some embodiments, a level is based on counts that are raw. In some embodiments, a level is associated with an uncertainty value (e.g., a standard deviation, a MAD). In some embodiments, a level is represented by a Z-score or p-value.

A level for one or more portions is synonymous with a “genomic section level” herein. The term “level” as used herein is sometimes synonymous with the term “elevation.” A determination of the meaning of the term “level” can be determined from the context in which it is used. For example, the term “level,” when used in the context of portions, profiles, reads and/or counts often means an elevation. The term “level,” when used in the context of a substance or composition (e.g., level of RNA, plexing level) often refers to an amount. The term “level,” when used in the context of uncertainty (e.g., level of error, level of confidence, level of deviation, level of uncertainty) often refers to an amount.

Normalized or non-normalized counts for two or more levels (e.g., two or more levels in a profile) can sometimes be mathematically manipulated (e.g., added, multiplied, averaged, normalized, the like or combination thereof) according to levels. For example, normalized or non-normalized counts for two or more levels can be normalized according to one, some or all of the levels in a profile. In some embodiments, normalized or non-normalized counts of all levels in a profile are normalized according to one level in the profile. In some embodiments, normalized or non-normalized counts of a first level in a profile are normalized according to normalized or non-normalized counts of a second level in the profile.

Non-limiting examples of a level (e.g., a first level, a second level) are a level for a set of portions comprising processed counts, a level for a set of portions comprising a mean, median or average of counts, a level for a set of portions comprising normalized counts, the like or any combination thereof. In some embodiments, a first level and a second level in a profile are derived from counts of portions mapped to the same chromosome. In some embodiments, a first level and a second level in a profile are derived from counts of portions mapped to different chromosomes.

In some embodiments, a level is determined from normalized or non-normalized counts mapped to one or more portions. In some embodiments, a level is determined from normalized or non-normalized counts mapped to two or more portions, where the normalized counts for each portion often are about the same. There can be variation in counts (e.g., normalized counts) in a set of portions for a level. In a set of portions for a level there can be one or more portions having counts that are significantly different than in other portions of the set (e.g., peaks and/or dips). Any suitable number of normalized or non-normalized counts associated with any suitable number of portions can define a level.

In some embodiments, one or more levels can be determined from normalized or non-normalized counts of all or some of the portions of a genome. Often a level can be determined from all or some of the normalized or non-normalized counts of a chromosome, or part thereof. In some embodiments, two or more counts derived from two or more portions (e.g., a set of portions) determine a level. In some embodiments, two or more counts (e.g., counts from two or more portions) determine a level. In some embodiments, counts from 2 to about 100,000 portions determine a level. In some embodiments, counts from 2 to about 50,000, 2 to about 40,000, 2 to about 30,000, 2 to about 20,000, 2 to about 10,000, 2 to about 5000, 2 to about 2500, 2 to about 1250, 2 to about 1000, 2 to about 500, 2 to about 250, 2 to about 100 or 2 to about 60 portions determine a level. In some embodiments, counts from about 10 to about 50 portions determine a level. In some embodiments, counts from about 20 to about 40 or more portions determine a level. In some embodiments, a level comprises counts from about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, 55, 60 or more portions. In some embodiments, a level corresponds to a set of portions (e.g., a set of portions of a reference genome, a set of portions of a chromosome or a set of portions of a part of a chromosome).

In some embodiments, a level is determined for normalized or non-normalized counts of portions that are contiguous. In some embodiments, portions (e.g., a set of portions) that are contiguous represent neighboring regions of a genome or neighboring regions of a chromosome or gene. For example, two or more contiguous portions, when aligned by merging the portions end to end, can represent a sequence assembly of a DNA sequence longer than each portion. For example, two or more contiguous portions can represent of an intact genome, chromosome, gene, intron, exon or part thereof. In some embodiments, a level is determined from a collection (e.g., a set) of contiguous portions and/or non-contiguous portions.

### Data Processing and Normalization

Mapped sequence reads that have been counted are referred to herein as raw data, since the data represents unmanipulated counts (e.g., raw counts). In some embodiments, sequence read data in a data set can be processed further (e.g., mathematically and/or statistically manipulated) and/or displayed to facilitate providing an outcome. In certain embodiments, data sets, including larger data sets, may benefit from pre-processing to facilitate further analysis. Pre-processing of data sets sometimes involves removal of redundant and/or uninformative portions or portions of a reference genome (e.g., portions of a reference genome with uninformative data, redundant mapped reads, portions with zero median counts, over represented or underrepresented sequences). Without being limited by theory, data processing and/or preprocessing may (i) remove noisy data, (ii) remove uninformative data, (iii) remove redundant data, (iv) reduce the complexity of larger data sets, and/or (v) facilitate transformation of the data from one form into one or more other forms. The terms “pre-processing” and “processing” when utilized with respect to data or data sets are collectively referred to herein as “processing.” Processing can render data more amenable to further analysis, and can generate an outcome in some embodiments. In some embodiments, one or more or all processing methods (e.g., normalization methods, portion filtering, mapping, validation, the like or combinations thereof) are performed by a processor, a microprocessor, a computer, in conjunction with memory and/or by a microprocessor-controlled apparatus.

The term “noisy data” as used herein refers to (a) data that has a significant variance between data points when analyzed or plotted, (b) data that has a significant standard deviation (e.g., greater than 3 standard deviations), (c) data that has a significant standard error of the mean, the like, and combinations of the foregoing. Noisy data sometimes occurs due to the quantity and/or quality of starting material (e.g., nucleic acid sample), and sometimes occurs as part of processes for preparing or replicating DNA used to generate sequence reads. In certain embodiments, noise results from certain sequences being overrepresented when prepared using PCR-based methods. Methods described herein can reduce or eliminate the contribution of noisy data, and therefore reduce the effect of noisy data on the provided outcome.

The terms “uninformative data,” “uninformative portions of a reference genome,” and “uninformative portions” as used herein refer to portions, or data derived therefrom, having a numerical value that is significantly different from a predetermined threshold value or falls outside a predetermined cutoff range of values. The terms “threshold” and “threshold value” herein refer to any number that is calculated using a qualifying data set and serves as a limit of diagnosis of a genetic variation or genetic alteration (e.g., a copy number alteration, an aneuploidy, a microduplication, a microdeletion, a chromosomal aberration, and the like). In certain embodiments, a threshold is exceeded by results obtained by methods described herein and a subject is diagnosed with a copy number alteration. A threshold value or range of values often is calculated by mathematically and/or statistically manipulating sequence read data (e.g., from a reference and/or subject), in some embodiments, and in certain embodiments, sequence read data manipulated to generate a threshold value or range of values is sequence read data (e.g., from a reference and/or subject). In some embodiments, an uncertainty value is determined. An uncertainty value generally is a measure of variance or error and can be any suitable measure of variance or error. In some embodiments, an uncertainty value is a standard deviation, standard error, calculated variance, p-value, or mean absolute deviation (MAD). In some embodiments, an uncertainty value can be calculated according to a formula described herein.

Any suitable procedure can be utilized for processing data sets described herein. Non-limiting examples of procedures suitable for use for processing data sets include filtering, normalizing, weighting, monitoring peak heights, monitoring peak areas, monitoring peak edges, peak level analysis, peak width analysis, peak edge location analysis, peak lateral tolerances, determining area ratios, mathematical processing of data, statistical processing of data, application of statistical algorithms, analysis with fixed variables, analysis with optimized variables, plotting data to identify patterns or trends for additional processing, the like and combinations of the foregoing. In some embodiments, data sets are processed based on various features (e.g., GC content, redundant mapped reads, centromere regions, telomere regions, the like and combinations thereof) and/or variables (e.g., subject gender, subject age, subject ploidy, percent contribution of cancer cell nucleic acid, fetal gender, maternal age, maternal ploidy, percent contribution of fetal nucleic acid, the like or combinations thereof). In certain embodiments, processing data sets as described herein can reduce the complexity and/or dimensionality of large and/or complex data sets. A non-limiting example of a complex data set includes sequence read data generated from one or more test subjects and a plurality of reference subjects of different ages and ethnic backgrounds. In some embodiments, data sets can include from thousands to millions, or millions to billions of sequence reads for each test and/or reference subject.

Data processing can be performed in any number of steps, in certain embodiments. For example, data may be processed using only a single processing procedure in some embodiments, and in certain embodiments, data may be processed using 1 or more, 5 or more, 10 or more or 20 or more processing steps (e.g., 1 or more processing steps, 2 or more processing steps, 3 or more processing steps, 4 or more processing steps, 5 or more processing steps, 6 or more processing steps, 7 or more processing steps, 8 or more processing steps, 9 or more processing steps, 10 or more processing steps, 11 or more processing steps, 12 or more processing steps, 13 or more processing steps, 14 or more processing steps, 15 or more processing steps, 16 or more processing steps, 17 or more processing steps, 18 or more processing steps, 19 or more processing steps, or 20 or more processing steps). In some embodiments, processing steps may be the same step repeated two or more times (e.g., filtering two or more times, normalizing two or more times), and in certain embodiments, processing steps may be two or more different processing steps (e.g., filtering, normalizing; normalizing, monitoring peak heights and edges; filtering, normalizing, normalizing to a reference, statistical manipulation to determine p-values, and the like), carried out simultaneously or sequentially. In some embodiments, any suitable number and/or combination of the same or different processing steps can be utilized to process sequence read data to facilitate providing an outcome. In certain embodiments, processing data sets by the criteria described herein may reduce the complexity and/or dimensionality of a data set.

In some embodiments, one or more processing steps can comprise one or more normalization steps. Normalization can be performed by a suitable method described herein or known in the art. In certain embodiments, normalization comprises adjusting values measured on different scales to a notionally common scale. In certain embodiments, normalization comprises a sophisticated mathematical adjustment to bring probability distributions of adjusted values into alignment. In some embodiments, normalization comprises aligning distributions to a normal distribution. In certain embodiments, normalization comprises mathematical adjustments that allow comparison of corresponding normalized values for different datasets in a way that eliminates the effects of certain gross influences (e.g., error and anomalies). In certain embodiments, normalization comprises scaling. Normalization sometimes comprises division of one or more data sets by a predetermined variable or formula. Normalization sometimes comprises subtraction of one or more data sets by a predetermined variable or formula. Non-limiting examples of normalization methods include portion-wise normalization, normalization by GC content, median count (median bin count, median portion count) normalization, linear and nonlinear least squares regression, locally estimated scatterplot smoothing (LOESS), GC LOESS, LOWESS (locally weighted scatterplot smoothing), principal component normalization, repeat masking (RM), GC-normalization and repeat masking (GCRM), conditional quantile normalization (cQn), and/or combinations thereof. In some embodiments, the determination of a presence or absence of a copy number alteration (e.g., an aneuploidy, a microduplication, a microdeletion) utilizes a normalization method (e.g., portion-wise normalization, normalization by GC content, median count (median bin count, median portion count) normalization, linear and nonlinear least squares regression, LOESS, GC LOESS, LOWESS (locally weighted scatterplot smoothing), principal component normalization, repeat masking (RM), GC-normalization and repeat masking (GCRM), cQn, a normalization method known in the art and/or a combination thereof). Described in greater detail hereafter are certain examples of normalization processes that can be utilized, such as LOESS normalization, principal component normalization, and hybrid normalization methods, for example. Aspects of certain normalization processes also are described, for example, in International Patent Application Publication No. WO 2013/052913 and International Patent Application Publication No. WO 2015/051163, the entire content of which is incorporated herein by reference for all purposes.

Any suitable number of normalizations can be used. In some embodiments, data sets can be normalized 1 or more, 5 or more, 10 or more or even 20 or more times. Data sets can be normalized to values (e.g., normalizing value) representative of any suitable feature or variable (e.g., sample data, reference data, or both). Non-limiting examples of types of data normalizations that can be used include normalizing raw count data for one or more selected test or reference portions to the total number of counts mapped to the chromosome or the entire genome on which the selected portion or sections are mapped; normalizing raw count data for one or more selected portions to a median reference count for one or more portions or the chromosome on which a selected portion is mapped; normalizing raw count data to previously normalized data or derivatives thereof; and normalizing previously normalized data to one or more other predetermined normalization variables. Normalizing a data set sometimes has the effect of isolating statistical error, depending on the feature or property selected as the predetermined normalization variable. Normalizing a data set sometimes also allows comparison of data characteristics of data having different scales, by bringing the data to a common scale (e.g., predetermined normalization variable). In some embodiments, one or more normalizations to a statistically derived value can be utilized to minimize data differences and diminish the importance of outlying data. Normalizing portions, or portions of a reference genome, with respect to a normalizing value sometimes is referred to as “portion-wise normalization.”

In certain embodiments, a processing step can comprise one or more mathematical and/or statistical manipulations. Any suitable mathematical and/or statistical manipulation, alone or in combination, may be used to analyze and/or manipulate a data set described herein. Any suitable number of mathematical and/or statistical manipulations can be used. In some embodiments, a data set can be mathematically and/or statistically manipulated 1 or more, 5 or more, 10 or more or 20 or more times. Non-limiting examples of mathematical and statistical manipulations that can be used include addition, subtraction, multiplication, division, algebraic functions, least squares estimators, curve fitting, differential equations, rational polynomials, double polynomials, orthogonal polynomials, z-scores, p-values, chi values, phi values, analysis of peak levels, determination of peak edge locations, calculation of peak area ratios, analysis of median chromosomal level, calculation of mean absolute deviation, sum of squared residuals, mean, standard deviation, standard error, the like or combinations thereof. A mathematical and/or statistical manipulation can be performed on all or a portion of sequence read data, or processed products thereof. Non-limiting examples of data set variables or features that can be statistically manipulated include raw counts, filtered counts, normalized counts, peak heights, peak widths, peak areas, peak edges, lateral tolerances, P-values, median levels, mean levels, count distribution within a genomic region, relative representation of nucleic acid species, the like or combinations thereof.

In some embodiments, a processing step can comprise the use of one or more statistical algorithms. Any suitable statistical algorithm, alone or in combination, may be used to analyze and/or manipulate a data set described herein. Any suitable number of statistical algorithms can be used. In some embodiments, a data set can be analyzed using 1 or more, 5 or more, 10 or more or 20 or more statistical algorithms. Non-limiting examples of statistical algorithms suitable for use with methods described herein include principal component analysis, decision trees, counternulls, multiple comparisons, omnibus test, Behrens-Fisher problem, bootstrapping, Fisher's method for combining independent tests of significance, null hypothesis, type I error, type II error, exact test, one-sample Z test, two-sample Z test, one-sample t-test, paired t-test, two-sample pooled t-test having equal variances, two-sample unpooled t-test having unequal variances, one-proportion z-test, two-proportion z-test pooled, two-proportion z-test unpooled, one-sample chi-square test, two-sample F test for equality of variances, confidence interval, credible interval, significance, meta analysis, simple linear regression, robust linear regression, the like or combinations of the foregoing. Non-limiting examples of data set variables or features that can be analyzed using statistical algorithms include raw counts, filtered counts, normalized counts, peak heights, peak widths, peak edges, lateral tolerances, P-values, median levels, mean levels, count distribution within a genomic region, relative representation of nucleic acid species, the like or combinations thereof.

In certain embodiments, a data set can be analyzed by utilizing multiple (e.g., 2 or more) statistical algorithms (e.g., least squares regression, principal component analysis, linear discriminant analysis, quadratic discriminant analysis, bagging, neural networks, support vector machine models, random forests, classification tree models, K-nearest neighbors, logistic regression and/or smoothing) and/or mathematical and/or statistical manipulations (e.g., referred to herein as manipulations). The use of multiple manipulations can generate an N-dimensional space that can be used to provide an outcome, in some embodiments. In certain embodiments, analysis of a data set by utilizing multiple manipulations can reduce the complexity and/or dimensionality of the data set. For example, the use of multiple manipulations on a reference data set can generate an N-dimensional space (e.g., probability plot) that can be used to represent the presence or absence of a genetic variation/genetic alteration and/or copy number alteration, depending on the status of the reference samples (e.g., positive or negative for a selected copy number alteration). Analysis of test samples using a substantially similar set of manipulations can be used to generate an N-dimensional point for each of the test samples. The complexity and/or dimensionality of a test subject data set sometimes is reduced to a single value or N-dimensional point that can be readily compared to the N-dimensional space generated from the reference data. Test sample data that fall within the N-dimensional space populated by the reference subject data are indicative of a genetic status substantially similar to that of the reference subjects. Test sample data that fall outside of the N-dimensional space populated by the reference subject data are indicative of a genetic status substantially dissimilar to that of the reference subjects. In some embodiments, references are euploid or do not otherwise have a genetic variation/genetic alteration and/or copy number alteration and/or medical condition.

After data sets have been counted, optionally filtered, normalized, and optionally weighted, the processed data sets can be further manipulated by one or more filtering and/or normalizing and/or weighting procedures, in some embodiments. A data set that has been further manipulated by one or more filtering and/or normalizing and/or weighting procedures can be used to generate a profile, in certain embodiments. The one or more filtering and/or normalizing and/or weighting procedures sometimes can reduce data set complexity and/or dimensionality, in some embodiments. An outcome can be provided based on a data set of reduced complexity and/or dimensionality. In some embodiments, a profile plot of processed data further manipulated by weighting, for example, is generated to facilitate classification and/or providing an outcome. An outcome can be provided based on a profile plot of weighted data, for example.

Filtering or weighting of portions can be performed at one or more suitable points in an analysis. For example, portions may be filtered or weighted before or after sequence reads are mapped to portions of a reference genome. Portions may be filtered or weighted before or after an experimental bias for individual genome portions is determined in some embodiments. In certain embodiments, portions may be filtered or weighted before or after levels are calculated.

After data sets have been counted, optionally filtered, normalized, and optionally weighted, the processed data sets can be manipulated by one or more mathematical and/or statistical (e.g., statistical functions or statistical algorithm) manipulations, in some embodiments. In certain embodiments, processed data sets can be further manipulated by calculating Z-scores for one or more selected portions, chromosomes, or portions of chromosomes. In some embodiments, processed data sets can be further manipulated by calculating P-values. In certain embodiments, mathematical and/or statistical manipulations include one or more assumptions pertaining to ploidy and/or fraction of a minority species (e.g., fraction of cancer cell nucleic acid; fetal fraction). In some embodiments, a profile plot of processed data further manipulated by one or more statistical and/or mathematical manipulations is generated to facilitate classification and/or providing an outcome. An outcome can be provided based on a profile plot of statistically and/or mathematically manipulated data. An outcome provided based on a profile plot of statistically and/or mathematically manipulated data often includes one or more assumptions pertaining to ploidy and/or fraction of a minority species (e.g., fraction of cancer cell nucleic acid; fetal fraction).

In some embodiments, analysis and processing of data can include the use of one or more assumptions. A suitable number or type of assumptions can be utilized to analyze or process a data set. Non-limiting examples of assumptions that can be used for data processing and/or analysis include subject ploidy, cancer cell contribution, maternal ploidy, fetal contribution, prevalence of certain sequences in a reference population, ethnic background, prevalence of a selected medical condition in related family members, parallelism between raw count profiles from different patients and/or runs after GC-normalization and repeat masking (e.g., GCRM), identical matches represent PCR artifacts (e.g., identical base position), assumptions inherent in a nucleic acid quantification assay (e.g., fetal quantifier assay (FQA)), assumptions regarding twins (e.g., if 2 twins and only 1 is affected the effective fetal fraction is only 50% of the total measured fetal fraction (similarly for triplets, quadruplets and the like)), cell free DNA (e.g., cfDNA) uniformly covers the entire genome, the like and combinations thereof.

In those instances where the quality and/or depth of mapped sequence reads does not permit an outcome prediction of the presence or absence of a genetic variation/genetic alteration and/or copy number alteration at a desired confidence level (e.g., 95% or higher confidence level), based on the normalized count profiles, one or more additional mathematical manipulation algorithms and/or statistical prediction algorithms, can be utilized to generate additional numerical values useful for data analysis and/or providing an outcome. The term “normalized count profile” as used herein refers to a profile generated using normalized counts. Examples of methods that can be used to generate normalized counts and normalized count profiles are described herein. As noted, mapped sequence reads that have been counted can be normalized with respect to test sample counts or reference sample counts. In some embodiments, a normalized count profile can be presented as a plot.

Described in greater detail hereafter are non-limiting examples of processing steps and normalization methods that can be utilized, such as normalizing to a window (static or sliding), weighting, determining bias relationship, LOESS normalization, principal component normalization, hybrid normalization, generating a profile and performing a comparison.

### Normalizing to a Window (Static or Sliding)

In certain embodiments, a processing step comprises normalizing to a static window, and in some embodiments, a processing step comprises normalizing to a moving or sliding window. The term “window” as used herein refers to one or more portions chosen for analysis, and sometimes is used as a reference for comparison (e.g., used for normalization and/or other mathematical or statistical manipulation). The term “normalizing to a static window” as used herein refers to a normalization process using one or more portions selected for comparison between a test subject and reference subject data set. In some embodiments, the selected portions are utilized to generate a profile. A static window generally includes a predetermined set of portions that do not change during manipulations and/or analysis. The terms “normalizing to a moving window” and “normalizing to a sliding window” as used herein refer to normalizations performed to portions localized to the genomic region (e.g., immediate surrounding portions, adjacent portion or sections, and the like) of a selected test portion, where one or more selected test portions are normalized to portions immediately surrounding the selected test portion. In certain embodiments, the selected portions are utilized to generate a profile. A sliding or moving window normalization often includes repeatedly moving or sliding to an adjacent test portion, and normalizing the newly selected test portion to portions immediately surrounding or adjacent to the newly selected test portion, where adjacent windows have one or more portions in common. In certain embodiments, a plurality of selected test portions and/or chromosomes can be analyzed by a sliding window process.

In some embodiments, normalizing to a sliding or moving window can generate one or more values, where each value represents normalization to a different set of reference portions selected from different regions of a genome (e.g., chromosome). In certain embodiments, the one or more values generated are cumulative sums (e.g., a numerical estimate of the integral of the normalized count profile over the selected portion, domain (e.g., part of chromosome), or chromosome). The values generated by the sliding or moving window process can be used to generate a profile and facilitate arriving at an outcome. In some embodiments, cumulative sums of one or more portions can be displayed as a function of genomic position. Moving or sliding window analysis sometimes is used to analyze a genome for the presence or absence of microdeletions and/or microduplications. In certain embodiments, displaying cumulative sums of one or more portions is used to identify the presence or absence of regions of copy number alteration (e.g., microdeletion, microduplication).

### Weighting

In some embodiments, a processing step comprises a weighting. The terms “weighted,” “weighting” or “weight function” or grammatical derivatives or equivalents thereof, as used herein, refer to a mathematical manipulation of a portion or all of a data set sometimes utilized to alter the influence of certain data set features or variables with respect to other data set features or variables (e.g., increase or decrease the significance and/or contribution of data contained in one or more portions or portions of a reference genome, based on the quality or usefulness of the data in the selected portion or portions of a reference genome). A weighting function can be used to increase the influence of data with a relatively small measurement variance, and/or to decrease the influence of data with a relatively large measurement variance, in some embodiments. For example, portions of a reference genome with underrepresented or low-quality sequence data can be “down weighted” to minimize the influence on a data set, whereas selected portions of a reference genome can be “up weighted” to increase the influence on a data set. A non-limiting example of a weighting function is [1/(standard deviation) 2]. Weighting portions sometimes removes portion dependencies. In some embodiments, one or more portions are weighted by an eigen function (e.g., an eigenfunction). In some embodiments, an eigen function comprises replacing portions with orthogonal eigen-portions. A weighting step sometimes is performed in a manner substantially similar to a normalizing step. In some embodiments, a data set is adjusted (e.g., divided, multiplied, added, subtracted) by a predetermined variable (e.g., weighting variable). In some embodiments, a data set is divided by a predetermined variable (e.g., weighting variable). A predetermined variable (e.g., minimized target function, Phi) often is selected to weigh different parts of a data set differently (e.g., increase the influence of certain data types while decreasing the influence of other data types).

### Bias Relationships

In some embodiments, a processing step comprises determining a bias relationship. For example, one or more relationships may be generated between local genome bias estimates and bias frequencies. The term “relationship” as use herein refers to a mathematical and/or a graphical relationship between two or more variables or values. A relationship can be generated by a suitable mathematical and/or graphical process. Non-limiting examples of a relationship include a mathematical and/or graphical representation of a function, a correlation, a distribution, a linear or non-linear equation, a line, a regression, a fitted regression, the like or a combination thereof. Sometimes a relationship comprises a fitted relationship. In some embodiments, a fitted relationship comprises a fitted regression. Sometimes a relationship comprises two or more variables or values that are weighted. In some embodiments, a relationship comprises a fitted regression where one or more variables or values of the relationship a weighted. Sometimes a regression is fitted in a weighted fashion. Sometimes a regression is fitted without weighting. In certain embodiments, generating a relationship comprises plotting or graphing.

In certain embodiments, a relationship is generated between GC densities and GC density frequencies. In some embodiments, generating a relationship between (i) GC densities and (ii) GC density frequencies for a sample provides a sample GC density relationship. In some embodiments, generating a relationship between (i) GC densities and (ii) GC density frequencies for a reference provides a reference GC density relationship. In some embodiments, where local genome bias estimates are GC densities, a sample bias relationship is a sample GC density relationship, and a reference bias relationship is a reference GC density relationship. GC densities of a reference GC density relationship and/or a sample GC density relationship are often representations (e.g., mathematical or quantitative representation) of local GC content.

In some embodiments, a relationship between local genome bias estimates and bias frequencies comprises a distribution. In some embodiments, a relationship between local genome bias estimates and bias frequencies comprises a fitted relationship (e.g., a fitted regression). In some embodiments, a relationship between local genome bias estimates and bias frequencies comprises a fitted linear or non-linear regression (e.g., a polynomial regression). In certain embodiments, a relationship between local genome bias estimates and bias frequencies comprises a weighted relationship where local genome bias estimates and/or bias frequencies are weighted by a suitable process. In some embodiments, a weighted fitted relationship (e.g., a weighted fitting) can be obtained by a process comprising a quantile regression, parameterized distributions or an empirical distribution with interpolation. In certain embodiments, a relationship between local genome bias estimates and bias frequencies for a test sample, a reference or part thereof, comprises a polynomial regression where local genome bias estimates are weighted. In some embodiments, a weighed fitted model comprises weighting values of a distribution. Values of a distribution can be weighted by a suitable process. In some embodiments, values located near tails of a distribution are provided less weight than values closer to the median of the distribution. For example, for a distribution between local genome bias estimates (e.g., GC densities) and bias frequencies (e.g., GC density frequencies), a weight is determined according to the bias frequency for a given local genome bias estimate, where local genome bias estimates comprising bias frequencies closer to the mean of a distribution are provided greater weight than local genome bias estimates comprising bias frequencies further from the mean.

In some embodiments, a processing step comprises normalizing sequence read counts by comparing local genome bias estimates of sequence reads of a test sample to local genome bias estimates of a reference (e.g., a reference genome, or part thereof). In some embodiments, counts of sequence reads are normalized by comparing bias frequencies of local genome bias estimates of a test sample to bias frequencies of local genome bias estimates of a reference. In some embodiments, counts of sequence reads are normalized by comparing a sample bias relationship and a reference bias relationship, thereby generating a comparison.

Counts of sequence reads may be normalized according to a comparison of two or more relationships. In certain embodiments, two or more relationships are compared thereby providing a comparison that is used for reducing local bias in sequence reads (e.g., normalizing counts). Two or more relationships can be compared by a suitable method. In some embodiments, a comparison comprises adding, subtracting, multiplying and/or dividing a first relationship from a second relationship. In certain embodiments, comparing two or more relationships comprises a use of a suitable linear regression and/or a non-linear regression. In certain embodiments, comparing two or more relationships comprises a suitable polynomial regression (e.g., a 3^{rd} order polynomial regression). In some embodiments, a comparison comprises adding, subtracting, multiplying and/or dividing a first regression from a second regression. In some embodiments, two or more relationships are compared by a process comprising an inferential framework of multiple regressions. In some embodiments, two or more relationships are compared by a process comprising a suitable multivariate analysis. In some embodiments, two or more relationships are compared by a process comprising a basis function (e.g., a blending function, e.g., polynomial bases, Fourier bases, or the like), splines, a radial basis function and/or wavelets.

In certain embodiments, a distribution of local genome bias estimates comprising bias frequencies for a test sample and a reference is compared by a process comprising a polynomial regression where local genome bias estimates are weighted. In some embodiments, a polynomial regression is generated between (i) ratios, each of which ratios comprises bias frequencies of local genome bias estimates of a reference and bias frequencies of local genome bias estimates of a sample and (ii) local genome bias estimates. In some embodiments, a polynomial regression is generated between (i) a ratio of bias frequencies of local genome bias estimates of a reference to bias frequencies of local genome bias estimates of a sample and (ii) local genome bias estimates. In some embodiments, a comparison of a distribution of local genome bias estimates for reads of a test sample and a reference comprises determining a log ratio (e.g., a log 2 ratio) of bias frequencies of local genome bias estimates for the reference and the sample. In some embodiments, a comparison of a distribution of local genome bias estimates comprises dividing a log ratio (e.g., a log 2 ratio) of bias frequencies of local genome bias estimates for the reference by a log ratio (e.g., a log 2 ratio) of bias frequencies of local genome bias estimates for the sample.

Normalizing counts according to a comparison typically adjusts some counts and not others. Normalizing counts sometimes adjusts all counts and sometimes does not adjust any counts of sequence reads. A count for a sequence read sometimes is normalized by a process that comprises determining a weighting factor and sometimes the process does not include directly generating and utilizing a weighting factor. Normalizing counts according to a comparison sometimes comprises determining a weighting factor for each count of a sequence read. A weighting factor is often specific to a sequence read and is applied to a count of a specific sequence read. A weighting factor is often determined according to a comparison of two or more bias relationships (e.g., a sample bias relationship compared to a reference bias relationship). A normalized count is often determined by adjusting a count value according to a weighting factor. Adjusting a count according to a weighting factor sometimes includes adding, subtracting, multiplying and/or dividing a count for a sequence read by a weighting factor. A weighting factor and/or a normalized count sometimes are determined from a regression (e.g., a regression line). A normalized count is sometimes obtained directly from a regression line (e.g., a fitted regression line) resulting from a comparison between bias frequencies of local genome bias estimates of a reference (e.g., a reference genome) and a test sample. In some embodiments, each count of a read of a sample is provided a normalized count value according to a comparison of (i) bias frequencies of a local genome bias estimates of reads compared to (ii) bias frequencies of a local genome bias estimates of a reference. In certain embodiments, counts of sequence reads obtained for a sample are normalized and bias in the sequence reads is reduced.

### LOESS Normalization

In some embodiments, a processing step comprises a LOESS normalization. LOESS is a regression modeling method known in the art that combines multiple regression models in a k-nearest-neighbor-based meta-model. LOESS is sometimes referred to as a locally weighted polynomial regression. GC LOESS, in some embodiments, applies a LOESS model to the relationship between fragment count (e.g., sequence reads, counts) and GC composition for portions of a reference genome. Plotting a smooth curve through a set of data points using LOESS is sometimes called a LOESS curve, particularly when each smoothed value is given by a weighted quadratic least squares regression over the span of values of the y-axis scattergram criterion variable. For each point in a data set, the LOESS method fits a low-degree polynomial to a subset of the data, with explanatory variable values near the point whose response is being estimated. The polynomial is fitted using weighted least squares, giving more weight to points near the point whose response is being estimated and less weight to points further away. The value of the regression function for a point is then obtained by evaluating the local polynomial using the explanatory variable values for that data point. The LOESS fit is sometimes considered complete after regression function values have been computed for each of the data points. Many of the details of this method, such as the degree of the polynomial model and the weights, are flexible.

### Principal Component Analysis

In some embodiments, a processing step comprises a principal component analysis (PCA). In some embodiments, sequence read counts (e.g., sequence read counts of a test sample) is adjusted according to a principal component analysis (PCA). In some embodiments, a read density profile (e.g., a read density profile of a test sample) is adjusted according to a principal component analysis (PCA). A read density profile of one or more reference samples and/or a read density profile of a test subject can be adjusted according to a PCA. Removing bias from a read density profile by a PCA related process is sometimes referred to herein as adjusting a profile. A PCA can be performed by a suitable PCA method, or a variation thereof. Non-limiting examples of a PCA method include a canonical correlation analysis (CCA), a Karhunen-Loève transform (KLT), a Hotelling transform, a proper orthogonal decomposition (POD), a singular value decomposition (SVD) of X, an eigenvalue decomposition (EVD) of XTX, a factor analysis, an Eckart-Young theorem, a Schmidt-Mirsky theorem, empirical orthogonal functions (EOF), an empirical eigenfunction decomposition, an empirical component analysis, quasiharmonic modes, a spectral decomposition, an empirical modal analysis, the like, variations or combinations thereof. A PCA often identifies and/or adjusts for one or more biases in a read density profile. A bias identified and/or adjusted for by a PCA is sometimes referred to herein as a principal component. In some embodiments, one or more biases can be removed by adjusting a read density profile according to one or more principal component using a suitable method. A read density profile can be adjusted by adding, subtracting, multiplying and/or dividing one or more principal components from a read density profile. In some embodiments, one or more biases can be removed from a read density profile by subtracting one or more principal components from a read density profile. Although bias in a read density profile is often identified and/or quantitated by a PCA of a profile, principal components are often subtracted from a profile at the level of read densities. A PCA often identifies one or more principal components. In some embodiments, a PCA identifies a 1^{st}, 2^{nd}, 3^{rd}, 4^{th}, 5^{th}, 6^{th}, 7^{th}, 8^{th}, 9^{th}, and a 10^{th} or more principal components. In certain embodiments, 1, 2, 3, 4, 5, 6, 7, 8, 9, 10 or more principal components are used to adjust a profile. In certain embodiments, 5 principal components are used to adjust a profile. Often, principal components are used to adjust a profile in the order of appearance in a PCA. For example, where three principal components are subtracted from a read density profile, a 1^{st}, 2^{nd} and 3^{rd} principal component are used. Sometimes a bias identified by a principal component comprises a feature of a profile that is not used to adjust a profile. For example, a PCA may identify a copy number alteration (e.g., an aneuploidy, microduplication, microdeletion, deletion, translocation, insertion) and/or a gender difference as a principal component. Thus, in some embodiments, one or more principal components are not used to adjust a profile. For example, sometimes a 1^{st}, 2^{nd} and 4^{th} principal component are used to adjust a profile where a 3^{rd} principal component is not used to adjust a profile.

A principal component can be obtained from a PCA using any suitable sample or reference. In some embodiments, principal components are obtained from a test sample (e.g., a test subject). In some embodiments, principal components are obtained from one or more references (e.g., reference samples, reference sequences, a reference set). In certain instances, a PCA is performed on a median read density profile obtained from a training set comprising multiple samples resulting in the identification of a 1^{st} principal component and a 2^{nd} principal component. In some embodiments, principal components are obtained from a set of subjects devoid of a copy number alteration in question. In some embodiments, principal components are obtained from a set of known euploids. Principal components are often identified according to a PCA performed using one or more read density profiles of a reference (e.g., a training set). One or more principal components obtained from a reference are often subtracted from a read density profile of a test subject thereby providing an adjusted profile.

### Hybrid Normalization

In some embodiments, a processing step comprises a hybrid normalization method. A hybrid normalization method may reduce bias (e.g., GC bias), in certain instances. A hybrid normalization, in some embodiments, comprises (i) an analysis of a relationship of two variables (e.g., counts and GC content) and (ii) selection and application of a normalization method according to the analysis. A hybrid normalization, in certain embodiments, comprises (i) a regression (e.g., a regression analysis) and (ii) selection and application of a normalization method according to the regression. In some embodiments, counts obtained for a first sample (e.g., a first set of samples) are normalized by a different method than counts obtained from another sample (e.g., a second set of samples). In some embodiments, counts obtained for a first sample (e.g., a first set of samples) are normalized by a first normalization method and counts obtained from a second sample (e.g., a second set of samples) are normalized by a second normalization method. For example, in certain embodiments, a first normalization method comprises use of a linear regression and a second normalization method comprises use of a non-linear regression (e.g., a LOESS, GC-LOESS, LOWESS regression, LOESS smoothing).

In some embodiments, a hybrid normalization method is used to normalize sequence reads mapped to portions of a genome or chromosome (e.g., counts, mapped counts, mapped reads). In certain embodiments, raw counts are normalized and, in some embodiments, adjusted, weighted, filtered or previously normalized counts are normalized by a hybrid normalization method. In certain embodiments, levels or Z-scores are normalized. In some embodiments, counts mapped to selected portions of a genome or chromosome are normalized by a hybrid normalization approach. Counts can refer to a suitable measure of sequence reads mapped to portions of a genome, non-limiting examples of which include raw counts (e.g., unprocessed counts), normalized counts (e.g., normalized by LOESS, principal component, or a suitable method), portion levels (e.g., average levels, mean levels, median levels, or the like), Z-scores, the like, or combinations thereof. The counts can be raw counts or processed counts from one or more samples (e.g., a test sample, a sample from a pregnant female). In some embodiments, counts are obtained from one or more samples obtained from one or more subjects.

In some embodiments, a normalization method (e.g., the type of normalization method) is selected according to a regression (e.g., a regression analysis) and/or a correlation coefficient. A regression analysis refers to a statistical technique for estimating a relationship among variables (e.g., counts and GC content). In some embodiments, a regression is generated according to counts and a measure of GC content for each portion of multiple portions of a reference genome.

Any suitable measure of GC content can be used, non-limiting examples of which include a measure of guanine (G), cytosine (C), adenine (A), thymine (T), purine (GC), or pyrimidine (AT or ATU) content, melting temperature (Tm) (e.g., denaturation temperature, annealing temperature, hybridization temperature), a measure of free energy, the like or combinations thereof. A measure of guanine (G), cytosine (C), adenine (A), thymine (T), purine (GC), or pyrimidine (AT or ATU) content can be expressed as a ratio or a percentage. In some embodiments, any suitable ratio or percentage is used, non-limiting examples of which include GC/AT, GC/total nucleotide, GC/A, GC/T, AT/total nucleotide, AT/GC, AT/G, AT/C, G/A, C/A, G/T, G/A, G/AT, C/T, the like or combinations thereof. In some embodiments, a measure of GC content is a ratio or percentage of GC to total nucleotide content. In some embodiments, a measure of GC content is a ratio or percentage of GC to total nucleotide content for sequence reads mapped to a portion of reference genome. In certain embodiments, the GC content is determined according to and/or from sequence reads mapped to each portion of a reference genome and the sequence reads are obtained from a sample. In some embodiments, a measure of GC content is not determined according to and/or from sequence reads. In certain embodiments, a measure of GC content is determined for one or more samples obtained from one or more subjects.

In some embodiments, generating a regression comprises generating a regression analysis or a correlation analysis. A suitable regression can be used, non-limiting examples of which include a regression analysis, (e.g., a linear regression analysis), a goodness of fit analysis, a Pearson's correlation analysis, a rank correlation, a fraction of variance unexplained, Nash-Sutcliffe model efficiency analysis, regression model validation, proportional reduction in loss, root mean square deviation, the like or a combination thereof. In some embodiments, a regression line is generated. In certain embodiments, generating a regression comprises generating a linear regression. In certain embodiments, generating a regression comprises generating a non-linear regression (e.g., a LOESS regression, a LOWESS regression).

In some embodiments, a regression determines the presence or absence of a correlation (e.g., a linear correlation), for example between counts and a measure of GC content. In some embodiments, a regression (e.g., a linear regression) is generated and a correlation coefficient is determined. In some embodiments, a suitable correlation coefficient is determined, non-limiting examples of which include a coefficient of determination, an R² value, a Pearson's correlation coefficient, or the like.

In some embodiments, goodness of fit is determined for a regression (e.g., a regression analysis, a linear regression). Goodness of fit sometimes is determined by visual or mathematical analysis. An assessment sometimes includes determining whether the goodness of fit is greater for a non-linear regression or for a linear regression. In some embodiments, a correlation coefficient is a measure of a goodness of fit. In some embodiments, an assessment of a goodness of fit for a regression is determined according to a correlation coefficient and/or a correlation coefficient cutoff value. In some embodiments, an assessment of a goodness of fit comprises comparing a correlation coefficient to a correlation coefficient cutoff value. In some embodiments, an assessment of a goodness of fit for a regression is indicative of a linear regression. For example, in certain embodiments, a goodness of fit is greater for a linear regression than for a non-linear regression and the assessment of the goodness of fit is indicative of a linear regression. In some embodiments, an assessment is indicative of a linear regression and a linear regression is used to normalize the counts. In some embodiments, an assessment of a goodness of fit for a regression is indicative of a non-linear regression. For example, in certain embodiments, a goodness of fit is greater for a non-linear regression than for a linear regression and the assessment of the goodness of fit is indicative of a non-linear regression. In some embodiments, an assessment is indicative of a non-linear regression and a non-linear regression is used to normalize the counts.

In some embodiments, an assessment of a goodness of fit is indicative of a linear regression when a correlation coefficient is equal to or greater than a correlation coefficient cutoff. In some embodiments, an assessment of a goodness of fit is indicative of a non-linear regression when a correlation coefficient is less than a correlation coefficient cutoff. In some embodiments, a correlation coefficient cutoff is pre-determined. In some embodiments, a correlation coefficient cutoff is about 0.5 or greater, about 0.55 or greater, about 0.6 or greater, about 0.65 or greater, about 0.7 or greater, about 0.75 or greater, about 0.8 or greater, about 0.85 or greater, about 0.9 or greater, or about 0.95 or greater.

In some embodiments, a specific type of regression is selected (e.g., a linear or non-linear regression) and, after the regression is generated, counts are normalized by subtracting the regression from the counts. In some embodiments, subtracting a regression from the counts provides normalized counts with reduced bias (e.g., GC bias). In some embodiments, a linear regression is subtracted from the counts. In some embodiments, a non-linear regression (e.g., a LOESS, GC-LOESS, LOWESS regression) is subtracted from the counts. Any suitable method can be used to subtract a regression line from the counts. For example, if counts x are derived from portion i (e.g., a portion i) comprising a GC content of 0.5 and a regression line determines counts y at a GC content of 0.5, then x-y=normalized counts for portion i. In some embodiments, counts are normalized prior to and/or after subtracting a regression. In some embodiments, counts normalized by a hybrid normalization approach are used to generate levels, Z-scores, levels and/or profiles of a genome or a part thereof. In certain embodiments, counts normalized by a hybrid normalization approach are analyzed by methods described herein to determine the presence or absence of a genetic variation or genetic alteration (e.g., copy number alteration).

In some embodiments, a hybrid normalization method comprises filtering or weighting one or more portions before or after normalization. A suitable method of filtering portions, including methods of filtering portions (e.g., portions of a reference genome) described herein can be used. In some embodiments, portions (e.g., portions of a reference genome) are filtered prior to applying a hybrid normalization method. In some embodiments, only counts of sequencing reads mapped to selected portions (e.g., portions selected according to count variability) are normalized by a hybrid normalization. In some embodiments, counts of sequencing reads mapped to filtered portions of a reference genome (e.g., portions filtered according to count variability) are removed prior to utilizing a hybrid normalization method. In some embodiments, a hybrid normalization method comprises selecting or filtering portions (e.g., portions of a reference genome) according to a suitable method (e.g., a method described herein). In some embodiments, a hybrid normalization method comprises selecting or filtering portions (e.g., portions of a reference genome) according to an uncertainty value for counts mapped to each of the portions for multiple test samples. In some embodiments, a hybrid normalization method comprises selecting or filtering portions (e.g., portions of a reference genome) according to count variability. In some embodiments, a hybrid normalization method comprises selecting or filtering portions (e.g., portions of a reference genome) according to GC content, repetitive elements, repetitive sequences, introns, exons, the like or a combination thereof.

### Profiles

In some embodiments, a processing step comprises generating one or more profiles (e.g., profile plot) from various aspects of a data set or derivation thereof (e.g., product of one or more mathematical and/or statistical data processing steps known in the art and/or described herein). The term “profile” as used herein refers to a product of a mathematical and/or statistical manipulation of data that can facilitate identification of patterns and/or correlations in large quantities of data. A “profile” often includes values resulting from one or more manipulations of data or data sets, based on one or more criteria. A profile often includes multiple data points. Any suitable number of data points may be included in a profile depending on the nature and/or complexity of a data set. In certain embodiments, profiles may include 2 or more data points, 3 or more data points, 5 or more data points, 10 or more data points, 24 or more data points, 25 or more data points, 50 or more data points, 100 or more data points, 500 or more data points, 1000 or more data points, 5000 or more data points, 10,000 or more data points, or 100,000 or more data points.

In some embodiments, a profile is representative of the entirety of a data set, and in certain embodiments, a profile is representative of a part or subset of a data set. That is, a profile sometimes includes or is generated from data points representative of data that has not been filtered to remove any data, and sometimes a profile includes or is generated from data points representative of data that has been filtered to remove unwanted data. In some embodiments, a data point in a profile represents the results of data manipulation for a portion. In certain embodiments, a data point in a profile includes results of data manipulation for groups of portions. In some embodiments, groups of portions may be adjacent to one another, and in certain embodiments, groups of portions may be from different parts of a chromosome or genome.

Data points in a profile derived from a data set can be representative of any suitable data categorization. Non-limiting examples of categories into which data can be grouped to generate profile data points include: portions based on size, portions based on sequence features (e.g., GC content, AT content, position on a chromosome (e.g., short arm, long arm, centromere, telomere), and the like), levels of expression, chromosome, the like or combinations thereof. In some embodiments, a profile may be generated from data points obtained from another profile (e.g., normalized data profile renormalized to a different normalizing value to generate a renormalized data profile). In certain embodiments, a profile generated from data points obtained from another profile reduces the number of data points and/or complexity of the data set. Reducing the number of data points and/or complexity of a data set often facilitates interpretation of data and/or facilitates providing an outcome.

A profile (e.g., a genomic profile, a chromosome profile, a profile of a part of a chromosome) often is a collection of normalized or non-normalized counts for two or more portions. A profile often includes at least one level, and often comprises two or more levels (e.g., a profile often has multiple levels). A level generally is for a set of portions having about the same counts or normalized counts. Levels are described in greater detail herein. In certain embodiments, a profile comprises one or more portions, which portions can be weighted, removed, filtered, normalized, adjusted, averaged, derived as a mean, added, subtracted, processed or transformed by any combination thereof. A profile often comprises normalized counts mapped to portions defining two or more levels, where the counts are further normalized according to one of the levels by a suitable method. Often counts of a profile (e.g., a profile level) are associated with an uncertainty value.

A profile comprising one or more levels is sometimes padded (e.g., hole padding). Padding (e.g., hole padding) refers to a process of identifying and adjusting levels in a profile that are due to copy number alterations (e.g., microduplications or microdeletions in a patient's genome, maternal microduplications or microdeletions). In some embodiments, levels are padded that are due to microduplications or microdeletions in a tumor or a fetus. Microduplications or microdeletions in a profile can, in some embodiments, artificially raise or lower the overall level of a profile (e.g., a profile of a chromosome) leading to false positive or false negative determinations of a chromosome aneuploidy (e.g., a trisomy). In some embodiments, levels in a profile that are due to microduplications and/or deletions are identified and adjusted (e.g., padded and/or removed) by a process sometimes referred to as padding or hole padding.

A profile comprising one or more levels can include a first level and a second level. In some embodiments, a first level is different (e.g., significantly different) than a second level. In some embodiments, a first level comprises a first set of portions, a second level comprises a second set of portions and the first set of portions is not a subset of the second set of portions. In certain embodiments, a first set of portions is different than a second set of portions from which a first and second level are determined. In some embodiments, a profile can have multiple first levels that are different (e.g., significantly different, e.g., have a significantly different value) than a second level within the profile. In some embodiments, a profile comprises one or more first levels that are significantly different than a second level within the profile and one or more of the first levels are adjusted. In some embodiments, a first level within a profile is removed from the profile or adjusted (e.g., padded). A profile can comprise multiple levels that include one or more first levels significantly different than one or more second levels and often the majority of levels in a profile are second levels, which second levels are about equal to one another. In some embodiments, greater than 50%, greater than 60%, greater than 70%, greater than 80%, greater than 90% or greater than 95% of the levels in a profile are second levels.

A profile sometimes is displayed as a plot. For example, one or more levels representing counts (e.g., normalized counts) of portions can be plotted and visualized. Non-limiting examples of profile plots that can be generated include raw count (e.g., raw count profile or raw profile), normalized count, portion-weighted, z-score, p-value, area ratio versus fitted ploidy, median level versus ratio between fitted and measured minority species fraction, principal components, the like, or combinations thereof. Profile plots allow visualization of the manipulated data, in some embodiments. In certain embodiments, a profile plot can be utilized to provide an outcome (e.g., area ratio versus fitted ploidy, median level versus ratio between fitted and measured minority species fraction, principal components). The terms “raw count profile plot” or “raw profile plot” as used herein refer to a plot of counts in each portion in a region normalized to total counts in a region (e.g., genome, portion, chromosome, chromosome portions of a reference genome or a part of a chromosome). In some embodiments, a profile can be generated using a static window process, and in certain embodiments, a profile can be generated using a sliding window process.

A profile generated for a test subject sometimes is compared to a profile generated for one or more reference subjects, to facilitate interpretation of mathematical and/or statistical manipulations of a data set and/or to provide an outcome. In some embodiments, a profile is generated based on one or more starting assumptions, e.g., assumptions described herein. In certain embodiments, a test profile often centers around a predetermined value representative of the absence of a copy number alteration, and often deviates from a predetermined value in areas corresponding to the genomic location in which the copy number alteration is located in the test subject, if the test subject possessed the copy number alteration. In test subjects at risk for, or suffering from a medical condition associated with a copy number alteration, the numerical value for a selected portion is expected to vary significantly from the predetermined value for non-affected genomic locations. Depending on starting assumptions (e.g., fixed ploidy or optimized ploidy, fixed fraction of cancer cell nucleic acid or optimized fraction of cancer cell nucleic acid, fixed fetal fraction or optimized fetal fraction, or combinations thereof) the predetermined threshold or cutoff value or threshold range of values indicative of the presence or absence of a copy number alteration can vary while still providing an outcome useful for determining the presence or absence of a copy number alteration. In some embodiments, a profile is indicative of and/or representative of a phenotype.

In some embodiments, the use of one or more reference samples that are substantially free of a copy number alteration in question can be used to generate a reference count profile (e.g., a reference median count profile), which may result in a predetermined value representative of the absence of the copy number alteration, and often deviates from a predetermined value in areas corresponding to the genomic location in which the copy number alteration is located in the test subject, if the test subject possessed the copy number alteration. In test subjects at risk for, or suffering from a medical condition associated with a copy number alteration, the numerical value for the selected portion or sections is expected to vary significantly from the predetermined value for non-affected genomic locations. In certain embodiments, the use of one or more reference samples known to carry the copy number alteration in question can be used to generate a reference count profile (a reference median count profile), which may result in a predetermined value representative of the presence of the copy number alteration, and often deviates from a predetermined value in areas corresponding to the genomic location in which a test subject does not carry the copy number alteration. In test subjects not at risk for, or suffering from a medical condition associated with a copy number alteration, the numerical value for the selected portion or sections is expected to vary significantly from the predetermined value for affected genomic locations.

By way of a non-limiting example, normalized sample and/or reference count profiles can be obtained from raw sequence read data by (a) calculating reference median counts for selected chromosomes, portions or parts thereof from a set of references known not to carry a copy number alteration, (b) removal of uninformative portions from the reference sample raw counts (e.g., filtering); (c) normalizing the reference counts for all remaining portions of a reference genome to the total residual number of counts (e.g., sum of remaining counts after removal of uninformative portions of a reference genome) for the reference sample selected chromosome or selected genomic location, thereby generating a normalized reference subject profile; (d) removing the corresponding portions from the test subject sample and (e) normalizing the remaining test subject counts for one or more selected genomic locations to the sum of the residual reference median counts for the chromosome or chromosomes containing the selected genomic locations, thereby generating a normalized test subject profile. In certain embodiments, an additional normalizing step with respect to the entire genome, reduced by the filtered portions in (b), can be included between (c) and (d).

In some embodiments, a read density profile is determined. In some embodiments, a read density profile comprises at least one read density, and often comprises two or more read densities (e.g., a read density profile often comprises multiple read densities). In some embodiments, a read density profile comprises a suitable quantitative value (e.g., a mean, a median, a Z-score, or the like). A read density profile often comprises values resulting from one or more read densities. A read density profile sometimes comprises values resulting from one or more manipulations of read densities based on one or more adjustments (e.g., normalizations). In some embodiments, a read density profile comprises unmanipulated read densities. In some embodiments, one or more read density profiles are generated from various aspects of a data set comprising read densities, or a derivation thereof (e.g., product of one or more mathematical and/or statistical data processing steps known in the art and/or described herein). In certain embodiments, a read density profile comprises normalized read densities. In some embodiments, a read density profile comprises adjusted read densities. In certain embodiments, a read density profile comprises raw read densities (e.g., unmanipulated, not adjusted or normalized), normalized read densities, weighted read densities, read densities of filtered portions, z-scores of read densities, p-values of read densities, integral values of read densities (e.g., area under the curve), average, mean or median read densities, principal components, the like, or combinations thereof. Often read densities of a read density profile and/or a read density profile is associated with a measure of uncertainty (e.g., a MAD). In certain embodiments, a read density profile comprises a distribution of median read densities. In some embodiments, a read density profile comprises a relationship (e.g., a fitted relationship, a regression, or the like) of a plurality of read densities. For example, sometimes a read density profile comprises a relationship between read densities (e.g., read densities value) and genomic locations (e.g., portions, portion locations). In some embodiments, a read density profile is generated using a static window process, and in certain embodiments, a read density profile is generated using a sliding window process. In some embodiments, a read density profile is sometimes printed and/or displayed (e.g., displayed as a visual representation, e.g., a plot or a graph).

In some embodiments, a read density profile corresponds to a set of portions (e.g., a set of portions of a reference genome, a set of portions of a chromosome or a subset of portions of a part of a chromosome). In some embodiments, a read density profile comprises read densities and/or counts associated with a collection (e.g., a set, a subset) of portions. In some embodiments, a read density profile is determined for read densities of portions that are contiguous. In some embodiments, contiguous portions comprise gaps comprising regions of a reference sequence and/or sequence reads that are not included in a density profile (e.g., portions removed by a filtering). Sometimes portions (e.g., a set of portions) that are contiguous represent neighboring regions of a genome or neighboring regions of a chromosome or gene. For example, two or more contiguous portions, when aligned by merging the portions end to end, can represent a sequence assembly of a DNA sequence longer than each portion. For example, two or more contiguous portions can represent an intact genome, chromosome, gene, intron, exon or part thereof. Sometimes a read density profile is determined from a collection (e.g., a set, a subset) of contiguous portions and/or non-contiguous portions. In some cases, a read density profile comprises one or more portions, which portions can be weighted, removed, filtered, normalized, adjusted, averaged, derived as a mean, added, subtracted, processed or transformed by any combination thereof.

A read density profile is often determined for a sample and/or a reference (e.g., a reference sample). A read density profile is sometimes generated for an entire genome, one or more chromosomes, or for a part of a genome or a chromosome. In some embodiments, one or more read density profiles are determined for a genome or part thereof. In some embodiments, a read density profile is representative of the entirety of a set of read densities of a sample, and in certain embodiments, a read density profile is representative of a part or subset of read densities of a sample. That is, sometimes a read density profile comprises or is generated from read densities representative of data that has not been filtered to remove any data, and sometimes a read density profile includes or is generated from data points representative of data that has been filtered to remove unwanted data.

In some embodiments, a read density profile is determined for a reference (e.g., a reference sample, a training set). A read density profile for a reference is sometimes referred to herein as a reference profile. In some embodiments, a reference profile comprises a read densities obtained from one or more references (e.g., reference sequences, reference samples). In some embodiments, a reference profile comprises read densities determined for one or more (e.g., a set of) known euploid samples. In some embodiments, a reference profile comprises read densities of filtered portions. In some embodiments, a reference profile comprises read densities adjusted according to the one or more principal components.

At block 130, the processed sequence read data is analyzed using one or more bioinformatics subsystems. In certain instances, the one or more bioinformatics subsystems comprises one or more machine learning models. For example, the processed sequence read data may be analyzed using one or more bioinformatics subsystems (e.g., a Chromosomal Aberration Decision Tree (CADET)) to detect a genome event (e.g., a presence or absence of a genetic variation). The one or more bioinformatics subsystems may also comprise a comparator and a transformer. In some instances, decision analysis is performed by the one or more bioinformatics subsystems.

### Performing a Comparison

In some embodiments, the one or more bioinformatics subsystems comprise a comparator to preform a comparison (e.g., comparing a test profile to a reference profile). Two or more data sets, two or more relationships and/or two or more profiles can be compared by a suitable method. Non-limiting examples of statistical methods suitable for comparing data sets, relationships and/or profiles include Behrens-Fisher approach, bootstrapping, Fisher's method for combining independent tests of significance, Neyman-Pearson testing, confirmatory data analysis, exploratory data analysis, exact test, F-test, Z-test, T-test, calculating and/or comparing a measure of uncertainty, a null hypothesis, counternulls and the like, a chi-square test, omnibus test, calculating and/or comparing level of significance (e.g., statistical significance), a meta analysis, a multivariate analysis, a regression, simple linear regression, robust linear regression, the like or combinations of the foregoing. In certain embodiments, comparing two or more data sets, relationships and/or profiles comprises determining and/or comparing a measure of uncertainty. A “measure of uncertainty” as used herein refers to a measure of significance (e.g., statistical significance), a measure of error, a measure of variance, a measure of confidence, the like or a combination thereof. A measure of uncertainty can be a value (e.g., a threshold) or a range of values (e.g., an interval, a confidence interval, a Bayesian confidence interval, a threshold range). Non-limiting examples of a measure of uncertainty include p-values, a suitable measure of deviation (e.g., standard deviation, sigma, absolute deviation, mean absolute deviation, the like), a suitable measure of error (e.g., standard error, mean squared error, root mean squared error, the like), a suitable measure of variance, a suitable standard score (e.g., standard deviations, cumulative percentages, percentile equivalents, Z-scores, T-scores, R-scores, standard nine (stanine), percent in stanine, the like), the like or combinations thereof. In some embodiments, determining the level of significance comprises determining a measure of uncertainty (e.g., a p-value). In certain embodiments, two or more data sets, relationships and/or profiles can be analyzed and/or compared by utilizing multiple (e.g., 2 or more) statistical methods (e.g., least squares regression, principal component analysis, linear discriminant analysis, quadratic discriminant analysis, bagging, neural networks, support vector machine models, random forests, classification tree models, K-nearest neighbors, logistic regression and/or loss smoothing) and/or any suitable mathematical and/or statistical manipulations (e.g., referred to herein as manipulations).

In some embodiments, a processing step comprises a comparison of two or more profiles (e.g., two or more read density profiles). Comparing profiles may comprise comparing profiles generated for a selected region of a genome. For example, a test profile may be compared to a reference profile where the test and reference profiles were determined for a region of a genome (e.g., a reference genome) that is substantially the same region. Comparing profiles sometimes comprises comparing two or more subsets of portions of a profile (e.g., a read density profile). A subset of portions of a profile may represent a region of a genome (e.g., a chromosome, or region thereof). A profile (e.g., a read density profile) can comprise any amount of subsets of portions. Sometimes a profile (e.g., a read density profile) comprises two or more, three or more, four or more, or five or more subsets. In certain embodiments, a profile (e.g., a read density profile) comprises two subsets of portions where each portion represents regions of a reference genome that are adjacent. In some embodiments, a test profile can be compared to a reference profile where the test profile and reference profile both comprise a first subset of portions and a second subset of portions where the first and second subsets represent different regions of a genome. Some subsets of portions of a profile may comprise copy number alterations and other subsets of portions are sometimes substantially free of copy number alterations. Sometimes all subsets of portions of a profile (e.g., a test profile) are substantially free of a copy number alteration. Sometimes all subsets of portions of a profile (e.g., a test profile) comprise a copy number alteration. In some embodiments, a test profile can comprise a first subset of portions that comprise a copy number alteration and a second subset of portions that are substantially free of a copy number alteration.

In certain embodiments, comparing two or more profiles comprises determining and/or comparing a measure of uncertainty for two or more profiles. Profiles (e.g., read density profiles) and/or associated measures of uncertainty are sometimes compared to facilitate interpretation of mathematical and/or statistical manipulations of a data set and/or to provide an outcome. A profile (e.g., a read density profile) generated for a test subject sometimes is compared to a profile (e.g., a read density profile) generated for one or more references (e.g., reference samples, reference subjects, and the like). In some embodiments, an outcome is provided by comparing a profile (e.g., a read density profile) from a test subject to a profile (e.g., a read density profile) from a reference for a chromosome, portions or parts thereof, where a reference profile is obtained from a set of reference subjects known not to possess a copy number alteration (e.g., a reference). In some embodiments, an outcome is provided by comparing a profile (e.g., a read density profile) from a test subject to a profile (e.g., a read density profile) from a reference for a chromosome, portions or parts thereof, where a reference profile is obtained from a set of reference subjects known to possess a specific copy number alteration (e.g., a chromosome aneuploidy, a microduplication, a microdeletion).

In certain embodiments, a profile (e.g., a read density profile) of a test subject is compared to a predetermined value representative of the absence of a copy number alteration, and sometimes deviates from a predetermined value at one or more genomic locations (e.g., portions) corresponding to a genomic location in which a copy number alteration is located. For example, in test subjects (e.g., subjects at risk for, or suffering from a medical condition associated with a copy number alteration), profiles are expected to differ significantly from profiles of a reference (e.g., a reference sequence, reference subject, reference set) for selected portions when a test subject comprises a copy number alteration in question. Profiles (e.g., read density profiles) of a test subject are often substantially the same as profiles (e.g., read density profiles) of a reference (e.g., a reference sequence, reference subject, reference set) for selected portions when a test subject does not comprise a copy number alteration in question. Profiles (e.g., read density profiles) may be compared to a predetermined threshold and/or threshold range. The term “threshold” as used herein refers to any number that is calculated using a qualifying data set and serves as a limit of diagnosis of a copy number alteration (e.g., an aneuploidy, a microduplication, a microdeletion, and the like). In certain embodiments, a threshold is exceeded by results obtained by methods described herein and a subject is diagnosed with a copy number alteration. In some embodiments, a threshold value or range of values may be calculated by mathematically and/or statistically manipulating sequence read data (e.g., from a reference and/or subject). A predetermined threshold or threshold range of values indicative of the presence or absence of a copy number alteration can vary while still providing an outcome useful for determining the presence or absence of a copy number alteration. In certain embodiments, a profile (e.g., a read density profile) comprising normalized read densities and/or normalized counts is generated to facilitate classification and/or providing an outcome. An outcome can be provided based on a plot of a profile (e.g., a read density profile) comprising normalized counts (e.g., using a plot of such a read density profile).

### Decision Analysis

In some embodiments, a determination of an outcome (e.g., making a call) or a determination of the presence or absence of a copy number alteration (e.g., chromosome aneuploidy, microduplication, microdeletion) is made according to a decision analysis. Certain decision analysis features are described in International Patent Application Publication No. WO 2014/190286, the entire content of which is incorporated herein by reference for all purposes. For example, a decision analysis sometimes comprises applying one or more methods that produce one or more results, an evaluation of the results, and a series of decisions based on the results, evaluations and/or the possible consequences of the decisions and terminating at some juncture of the process where a final decision is made. In some embodiments, a decision analysis is a decision tree. A decision analysis, in some embodiments, comprises coordinated use of one or more processes (e.g., process steps, e.g., algorithms). A decision analysis can be performed by a person, a system, an apparatus, software (e.g., a subsystem), a computer, a processor (e.g., a microprocessor), the like or a combination thereof. In some embodiments, a decision analysis comprises a method of determining the presence or absence of a copy number alteration (e.g., chromosome aneuploidy, microduplication or microdeletion) with reduced false negative and reduced false positive determinations, compared to an instance in which no decision analysis is utilized (e.g., a determination is made directly from normalized counts). In some embodiments, a decision analysis comprises determining the presence or absence of a condition associated with one or more copy number alterations.

In some embodiments, a decision analysis comprises generating a profile for a genome or a region of a genome (e.g., a chromosome or part thereof). A profile can be generated by any suitable method, known or described herein. In some embodiments, a decision analysis comprises a segmenting process. Segmenting can modify and/or transform a profile thereby providing one or more decomposition renderings of a profile. A profile subjected to a segmenting process often is a profile of normalized counts mapped to portions in a reference genome or part thereof. As addressed herein, raw counts mapped to the portions can be normalized by one or more suitable normalization processes (e.g., LOESS, GC-LOESS, principal component normalization, or combination thereof) to generate a profile that is segmented as part of a decision analysis. A decomposition rendering of a profile is often a transformation of a profile. A decomposition rendering of a profile is sometimes a transformation of a profile into a representation of a genome, chromosome or part thereof.

In certain embodiments, a segmenting process utilized for the segmenting locates and identifies one or more levels within a profile that are different (e.g., substantially or significantly different) than one or more other levels within a profile. A level identified in a profile according to a segmenting process that is different than another level in the profile, and has edges that are different than another level in the profile, is referred to herein as a level for a discrete segment. A segmenting process can generate, from a profile of normalized counts or levels, a decomposition rendering in which one or more discrete segments can be identified. A discrete segment generally covers fewer portions than what is segmented (e.g., chromosome, chromosomes, autosomes).

In some embodiments, segmenting locates and identifies edges of discrete segments within a profile. In certain embodiments, one or both edges of one or more discrete segments are identified. For example, a segmentation process can identify the location (e.g., genomic coordinates, e.g., portion location) of the right and/or the left edges of a discrete segment in a profile. A discrete segment often comprises two edges. For example, a discrete segment can include a left edge and a right edge. In some embodiments, depending upon the representation or view, a left edge can be a 5′-edge and a right edge can be a 3′-edge of a nucleic acid segment in a profile. In some embodiments, a left edge can be a 3′-edge and a right edge can be a 5′-edge of a nucleic acid segment in a profile. Often the edges of a profile are known prior to segmentation and therefore, in some embodiments, the edges of a profile determine which edge of a level is a 5′-edge and which edge is 3′-edge. In some embodiments, one or both edges of a profile and/or discrete segment is an edge of a chromosome.

In some embodiments, the edges of a discrete segment are determined according to a decomposition rendering generated for a reference sample (e.g., a reference profile). In some embodiments, a null edge height distribution is determined according to a decomposition rendering of a reference profile (e.g., a profile of a chromosome or part thereof). In certain embodiments, the edges of a discrete segment in a profile are identified when the level of the discrete segment is outside a null edge height distribution. In some embodiments, the edges of a discrete segment in a profile are identified according to a Z-score calculated according to a decomposition rendering for a reference profile.

In some instances, segmenting generates two or more discrete segments (e.g., two or more fragmented levels, two or more fragmented segments) in a profile. In some embodiments, a decomposition rendering derived from a segmenting process is over-segmented or fragmented and comprises multiple discrete segments. Sometimes discrete segments generated by segmenting are substantially different and sometimes discrete segments generated by segmenting are substantially similar. Substantially similar discrete segments (e.g., substantially similar levels) often refers to two or more adjacent discrete segments in a segmented profile each having a level that differs by less than a predetermined level of uncertainty. In some embodiments, substantially similar discrete segments are adjacent to each other and are not separated by an intervening segment. In some embodiments, substantially similar discrete segments are separated by one or more smaller segments. In some embodiments, substantially similar discrete segments are separated by about 1 to about 20, about 1 to about 15, about 1 to about 10 or about 1 to about 5 portions where one or more of the intervening portions have a level significantly different than the level of each of the substantially similar discrete segments. In some embodiments, the level of substantially similar discrete segments differs by less than about 3 times, less than about 2 times, less than about 1 time or less than about 0.5 times a level of uncertainty. Substantially similar discrete segments, in some embodiments, comprise a median level that differs by less than 3 MAD (e.g., less than 3 sigma), less than 2 MAD, less than 1 MAD or less than about 0.5 MAD, where a MAD is calculated from a median level of each of the segments. Substantially different discrete segments, in some embodiments, are not adjacent or are separated by 10 or more, 15 or more or 20 or more portions. Substantially different discrete segments generally have substantially different levels. In certain embodiments, substantially different discrete segments comprise levels that differ by more than about 2.5 times, more than about 3 times, more than about 4 times, more than about 5 times, more than about 6 times a level of uncertainty. Substantially different discrete segments, in some embodiments, comprise a median level that differs by more than 2.5 MAD (e.g., more than 2.5 sigma), more than 3 MAD, more than 4 MAD, more than about 5 MAD or more than about 6 MAD, where a MAD is calculated from a median level of each of the discrete segments.

In some embodiments, a segmentation process comprises determining (e.g., calculating) a level (e.g., a quantitative value, e.g., a mean or median level), a level of uncertainty (e.g., an uncertainty value), Z-score, Z-value, p-value, the like or combinations thereof for one or more discrete segments in a profile or part thereof. In some embodiments, a level (e.g., a quantitative value, e.g., a mean or median level), a level of uncertainty (e.g., an uncertainty value), Z-score, Z-value, p-value, the like or combinations thereof are determined (e.g., calculated) for a discrete segment.

Segmenting can be performed, in full or in part, by one or more decomposition generating processes. A decomposition generating process may provide, for example, a decomposition rendering of a profile. Any decomposition generating process described herein or known in the art may be used. Non-limiting examples of a decomposition generating process include circular binary segmentation (CBS) (see e.g., Olshen et al. (2004) Biostatistics 5 (4): 557-72; Venkatraman, E S, Olshen, A B (2007) Bioinformatics 23 (6): 657-63); Haar wavelet segmentation (see e.g., Haar, Alfred (1910) Mathematische Annalen 69 (3): 331-371); maximal overlap discrete wavelet transform (MODWT) (see e.g., Hsu et al. (2005) Biostatistics 6 (2): 211-226); stationary wavelet (SWT) (see e.g., Y. Wang and S. Wang (2007) International Journal of Bioinformatics Research and Applications 3 (2): 206-222); dual-tree complex wavelet transform (DTCWT) (see e.g., Nguyen et al. (2007) Proceedings of the 7th IEEE International Conference, Boston Mass., on Oct. 14-17, 2007, pages 137-144); maximum entropy segmentation, convolution with edge detection kernel, Jensen Shannon Divergence, Kullback-Leibler divergence, Binary Recursive Segmentation, a Fourier transform, the like or combinations thereof.

In some embodiments, segmenting is accomplished by a process that comprises one process or multiple sub-processes, non-limiting examples of which include a decomposition generating process, thresholding, leveling, smoothing, polishing, the like or combination thereof. Thresholding, leveling, smoothing, polishing and the like can be performed in conjunction with a decomposition generating process, for example.

In some embodiments, a decision analysis comprises identifying a candidate segment in a decomposition rendering. A candidate segment is determined as being the most significant discrete segment in a decomposition rendering. A candidate segment may be the most significant in terms of the number of portions covered by the segment and/or in terms of the absolute value of the level of normalized counts for the segment. A candidate segment sometimes is larger and sometimes substantially larger than other discrete segments in a decomposition rendering. A candidate segment can be identified by a suitable method. In some embodiments, a candidate segment is identified by an area under the curve (AUC) analysis. In certain embodiments, where a first discrete segment has a level and/or covers a number of portions substantially larger than for another discrete segment in a decomposition rendering, the first segment comprises a larger AUC. Where a level is analyzed for AUC, an absolute value of a level often is utilized (e.g., a level corresponding to normalized counts can have a negative value for a deletion and a positive value for a duplication). In certain embodiments, an AUC is determined as an absolute value of a calculated AUC (e.g., a resulting positive value). In certain embodiments, a candidate segment, once identified (e.g., by an AUC analysis or by a suitable method) and optionally after it is validated, is selected for a z-score calculation, or the like, to determine if the candidate segment represents a genetic variation or genetic alteration (e.g., an aneuploidy, microdeletion or microduplication).

In some embodiments, a decision analysis comprises a comparison. In some embodiments, a comparison comprises comparing at least two decomposition renderings. In some embodiments, a comparison comprises comparing at least two candidate segments. In certain embodiments, each of the at least two candidate segments is from a different decomposition rendering. For example, a first candidate segment can be from a first decomposition rendering and a second candidate segment can be from a second decomposition rendering. In some embodiments, a comparison comprises determining if two decomposition renderings are substantially the same or different. In some embodiments, a comparison comprises determining if two candidate segments are substantially the same or different. Two candidate segments can be determined as substantially the same or different by a suitable comparison method, non-limiting examples of which include by visual inspection, by comparing levels or Z-scores of the two candidate segments, by comparing the edges of the two candidate segments, by overlaying either the two candidate segments or their corresponding decomposition renderings, the like or combinations thereof.

### Transformations

As noted above, data sometimes is transformed from one form into another form. The terms “transformed,” “transformation,” and grammatical derivations or equivalents thereof, as used herein refer to an alteration of data from a physical starting material (e.g., test subject and/or reference subject sample nucleic acid) into a digital representation of the physical starting material (e.g., sequence read data), and in some embodiments, includes a further transformation into one or more numerical values or graphical representations of the digital representation that can be utilized to provide an outcome. In certain embodiments, the one or more numerical values and/or graphical representations of digitally represented data can be utilized to represent the appearance of a t′st subject's physical genome (e.g., virtually represent or visually represent the presence or absence of a genomic insertion, duplication or deletion; represent the presence or absence of a variation in the physical amount of a sequence associated with medical conditions). A virtual representation sometimes is further transformed into one or more numerical values or graphical representations of the digital representation of the starting material. These methods can transform physical starting material into a numerical value or graphical representation, or a representation of the physical appearance of a test subject's nucleic acid.

In some embodiments, transformation of a data set facilitates providing an outcome by reducing data complexity and/or data dimensionality. Data set complexity sometimes is reduced during the process of transforming a physical starting material into a virtual representation of the starting material (e.g., sequence reads representative of physical starting material). A suitable feature or variable can be utilized to reduce data set complexity and/or dimensionality. Non-limiting examples of features that can be chosen for use as a target feature for data processing include GC content, fetal gender prediction, fragment size (e.g., length of CCF fragments, reads or a suitable representation thereof (e.g., FRS)), fragment sequence, identification of a copy number alteration, identification of chromosomal aneuploidy, identification of particular genes or proteins, identification of cancer, diseases, inherited genes/traits, chromosomal abnormalities, a biological category, a chemical category, a biochemical category, a category of genes or proteins, a gene ontology, a protein ontology, co-regulated genes, cell signaling genes, cell cycle genes, proteins pertaining to the foregoing genes, gene variants, protein variants, co-regulated genes, co-regulated proteins, amino acid sequence, nucleotide sequence, protein structure data and the like, and combinations of the foregoing. Non-limiting examples of data set complexity and/or dimensionality reduction include; reduction of a plurality of sequence reads to profile plots, reduction of a plurality of sequence reads to numerical values (e.g., normalized values, Z-scores, p-values); reduction of multiple analysis methods to probability plots or single points; principal component analysis of derived quantities; and the like or combinations thereof.

### Classifications

Methods described herein can provide an outcome indicative of presence or absence of genomic instability for a test sample. Methods described herein sometimes provide an outcome indicative of a genotype and/or presence or absence of a genetic variation/alteration in a genomic region for a test sample (e.g., providing an outcome determinative of the presence or absence of a genetic variation). Methods described herein sometimes provide an outcome indicative of a phenotype and/or presence or absence of a medical condition for a test sample (e.g., providing an outcome determinative of the presence or absence of a medical condition and/or phenotype). An outcome often is part of a classification process, and a classification (e.g., classification of presence or absence of genomic instability, a genotype, a phenotype, a genetic variation and/or a medical condition for a test sample) sometimes is based on and/or includes an outcome. An outcome and/or classification sometimes is based on and/or includes a result of data processing for a test sample that facilitates determining presence or absence of genomic instability, a genotype, a phenotype, a genetic variation, genetic alteration, and/or a medical condition in a classification process (e.g., a statistic value (e.g., standard score (e.g., z-score)). An outcome and/or classification sometimes includes or is based on a score determinative of, or a call of, presence or absence of genomic instability, a genotype, a phenotype, a genetic variation, genetic alteration, and/or a medical condition. In certain embodiments, an outcome and/or classification includes a conclusion that predicts and/or determines presence or absence of genomic instability, a genotype, a phenotype, a genetic variation, genetic alteration, and/or a medical condition in a classification process. In some instances, the outcome and/or classification are the output of one or more machine learning models trained for predicting a presence or absence of genomic instability for a test sample.

A genotype and/or genetic variation often includes a gain, a loss and/or alteration of a region comprising one or more nucleotides (e.g., duplication, deletion, fusion, insertion, short tandem repeat (STR), mutation, single nucleotide alteration, reorganization, substitution or aberrant methylation) that results in a detectable change in the genome or genetic information for a test sample. A genotype and/or genetic variation often is in a particular genomic region (e.g., chromosome, portion of a chromosome (i.e., sub-chromosome region), STR, polymorphic region, translocated region, altered nucleotide sequence, the like or combinations of the foregoing). A genetic variation sometimes is a copy number alteration for a particular region, such as a trisomy or monosomy for chromosome region, or a microduplication or microdeletion event for a particular region (e.g., gain or loss of a region of about 10 megabases or less (e.g., about 9 megabases or less, 8 megabases or less, 7 megabases or less, 6 megabases or less, 5 megabases or less, 4 megabases or less, 3 megabases or less, 2 megabases or less or 1 megabase or less)), for example. A copy number alteration sometimes is expressed as having no copy or one, two, three or four or more copies of a particular region (e.g., chromosome, sub-chromosome, STR, microduplication or microdeletion region).

Presence or absence of genomic instability, a genotype, a phenotype, a genetic variation and/or a medical condition can be determined by transforming, analyzing and/or manipulating sequence reads that have been mapped to genomic portions (e.g., counts, counts of genomic portions of a reference genome). In certain embodiments, an outcome and/or classification is determined according to normalized counts, read densities, read density profiles, and the like, and can be determined by a method described herein. An outcome and/or classification sometimes includes one or more scores and/or calls that refer to the probability that genomic instability, or a particular genotype, phenotype, genetic variation, or medical condition, is present or absent for a test sample. The value of a score may be used to determine, for example, a variation, difference, or ratio of mapped sequence reads that may correspond to genomic instability, a genotype, phenotype, genetic variation, or medical condition. For example, calculating a positive score for genomic instability, or a selected genotype, phenotype, genetic variation, or medical condition, from a data set, with respect to a reference genome, can lead to a classification of genomic instability, or the genotype, phenotype, genetic variation, or medical condition, for a test sample.

Any suitable expression of an outcome and/or classification can be provided. An outcome and/or classification sometimes is based on and/or includes one or more numerical values generated using a processing method described herein in the context of one or more considerations of probability. Non-limiting examples of values that can be utilized include a sensitivity, specificity, standard deviation, median absolute deviation (MAD), measure of certainty, measure of confidence, measure of certainty or confidence that a value obtained for a test sample is inside or outside a particular range of values, measure of uncertainty, measure of uncertainty that a value obtained for a test sample is inside or outside a particular range of values, coefficient of variation (CV), confidence level, confidence interval (e.g., about 95% confidence interval), standard score (e.g., z-score), chi value, phi value, result of a t-test, p-value, ploidy value, fitted minority species fraction, area ratio, median level, the like or combination thereof. In some embodiments, an outcome and/or classification comprises a read density, a read density profile and/or a plot (e.g., a profile plot). In certain embodiments, multiple values are analyzed together, sometimes in a profile for such values (e.g., z-score profile, p-value profile, chi value profile, phi value profile, result of a t-test, value profile, the like, or combination thereof). A consideration of probability can facilitate determining whether a subject is at risk of having, or has, genomic instability, a genotype, phenotype, genetic variation and/or medical condition, and an outcome and/or classification determinative of the foregoing sometimes includes such a consideration.

In certain embodiments, an outcome and/or classification is based on and/or includes a conclusion that predicts and/or determines a risk or probability of the presence or absence of genomic instability, a genotype, phenotype, genetic variation and/or medical condition for a test sample. A conclusion sometimes is based on a value determined from a data analysis method described herein (e.g., a statistics value indicative of probability, certainty and/or uncertainty (e.g., standard deviation, median absolute deviation (MAD), measure of certainty, measure of confidence, measure of certainty or confidence that a value obtained for a test sample is inside or outside a particular range of values, measure of uncertainty, measure of uncertainty that a value obtained for a test sample is inside or outside a particular range of values, coefficient of variation (CV), confidence level, confidence interval (e.g., about 95% confidence interval), standard score (e.g., z-score), chi value, phi value, result of a t-test, p-value, sensitivity, specificity, the like or combination thereof). An outcome and/or classification sometimes is expressed in a laboratory test report (described in greater detail hereafter) for particular test sample as a probability (e.g., odds ratio, p-value), likelihood, or risk factor, associated with the presence or absence of genomic instability, a genotype, phenotype, genetic variation and/or medical condition. An outcome and/or classification for a test sample sometimes is provided as “positive” or “negative” with respect a genomic instability, or a particular genotype, phenotype, genetic variation and/or medical condition. For example, an outcome and/or classification sometimes is designated as “positive” in a laboratory test report for a particular test sample where presence of genomic instability, a genotype, phenotype, genetic variation and/or medical condition is determined, and sometimes an outcome and/or classification is designated as “negative” in a laboratory test report for a particular test sample where absence of genomic instability, a genotype, phenotype, genetic variation and/or medical condition is determined. An outcome and/or classification sometimes is determined and sometimes includes an assumption used in data processing.

An outcome and/or classification sometimes is based on or is expressed as a value in or out of a cluster, value over or under a threshold value, value within a range (e.g., a threshold range), and/or a value with a measure of variance or confidence. In some embodiments, an outcome and/or classification is based on or is expressed as a value above or below a predetermined threshold or cutoff value and/or a measure of uncertainty, confidence level or confidence interval associated with the value. In certain embodiments, a predetermined threshold or cutoff value is an expected level or an expected level range. In some embodiments, a value obtained for a test sample is a standard score (e.g., z-score), where presence of genomic instability, a genotype, phenotype, genetic variation and/or medical condition is determined when the absolute value of the score is greater than a particular score threshold (e.g., threshold between about 2 and about 5; between about 3 and about 4), and where the absence of genomic instability, a genotype, phenotype, genetic variation and/or medical condition is determined when the absolute value of the score is less than the particular score threshold. In certain embodiments, an outcome and/or classification is based on or is expressed as a value that falls within or outside a predetermined range of values (e.g., a threshold range) and the associated uncertainty or confidence level for that value being inside or outside the range. In some embodiments, an outcome and/or classification comprises a value that is equal to a predetermined value (e.g., equal to 1, equal to zero), or is equal to a value within a predetermined value range, and its associated uncertainty or confidence level for that value being equal or within or outside the range. An outcome and/or classification sometimes is graphically represented as a plot (e.g., profile plot). An outcome and/or classification sometimes comprises use of a reference value or reference profile, and sometimes a reference value or reference profile is obtained from one or more reference samples (e.g., reference sample(s) euploid for a selected part of a genome (e.g., region)).

In some embodiments, an outcome and/or classification is based on or includes use of a measure of uncertainty between a test value or profile and a reference value or profile for a selected region. In some embodiments, a determination of the presence or absence of genomic instability, a genotype, phenotype, genetic variation and/or medical condition is according to the number of deviations (e.g., sigma) between a test value or profile and a reference value or profile for a selected region (e.g., a chromosome, or part thereof). A measure of deviation often is an absolute value or absolute measure of deviation (e.g., mean absolute deviation or median absolute deviation (MAD)). In some embodiments, the presence of genomic instability, a genotype, phenotype, genetic variation and/or medical condition is determined when the number of deviations between a test value or profile and a reference value or profile is about 1 or greater (e.g., about 1.5, 2, 2.5, 2.6, 2.7, 2.8, 2.9, 3, 3.1, 3.2, 3.3, 3.4, 3.5, 3.6, 3.7, 3.8, 3.9, 4, 5 or 6 deviations or greater). In certain embodiments, presence of genomic instability, a genotype, phenotype, genetic variation and/or medical condition is determined when a test value or profile and a reference value or profile differ by about 2 to about 5 measures of deviation (e.g., sigma, MAD), or more than 3 measures of deviation (e.g., 3 sigma, 3 MAD). A deviation of greater than three between a test value or profile and a reference value or profile often is indicative of a non-euploid test subject (e.g., presence of a genetic variation (e.g., presence of trisomy, monosomy, microduplication, microdeletion) for a selected region. Test values or profiles significantly above a reference profile, which reference profile is indicative of euploidy, sometimes are determinative of a trisomy, sub-chromosome duplication or microduplication. Test values or profiles significantly below a reference profile, which reference profile is indicative of euploidy, sometimes are determinative of a monosomy, sub-chromosome deletion or microdeletion. In some embodiments, the absence of genomic instability, a genotype, phenotype, genetic variation and/or medical condition is determined when the number of deviations between a test value or profile and reference value or profile for a selected region of a genome is about 3.5 or less (e.g., about less than about 3.4, 3.3, 3.2, 3.1, 3, 2.9, 2.8, 2.7, 2.6, 2.5, 2.4, 2.3, 2.2, 2.1, 2, 1.9, 1.8, 1.7, 1.6, 1.5, 1.4, 1.3, 1.2, 1.1, 1 or less). In certain embodiments, absence of genomic instability, a genotype, phenotype, genetic variation and/or medical condition is determined when a test value or profile differs from a reference value or profile by less than three measures of deviation (e.g., 3 sigma, 3 MAD). In some embodiments, a measure of deviation of less than three between a test value or profile and reference value or profile (e.g., 3-sigma for standard deviation) often is indicative of a region that is euploid (e.g., absence of a genetic variation). A measure of deviation between a test value or profile for a test sample and a reference value or profile for one or more reference subjects can be plotted and visualized (e.g., z-score plot).

In some embodiments, an outcome and/or classification is determined according to a call zone. In certain embodiments, a call is made (e.g., a call determining presence or absence of genomic instability, a genotype, phenotype, genetic variation and/or medical condition) when a value (e.g., a profile, a read density profile and/or a measure of uncertainty) or collection of values falls within a pre-defined range (e.g., a zone, a call zone). In some embodiments, a call zone is defined according to a collection of values (e.g., profiles, read density profiles, measures or determination of probability and/or measures of uncertainty) obtained from a particular group of samples. In certain embodiments, a call zone is defined according to a collection of values that are derived from the same chromosome or part thereof. In some embodiments, a call zone for determining presence or absence of genomic instability, a genotype, phenotype, genetic variation and/or medical condition is defined according a measure of uncertainty (e.g., high level of confidence or low measure of uncertainty) and/or a quantification of a minority nucleic acid species (e.g., about 1% minority species or greater (e.g., about 2, 3, 4, 5, 6, 7, 8, 9, 10% or more minority nucleic acid species)) determined for a test sample. A minority nucleic acid species quantification sometimes is a fraction or percent of cancer cell nucleic acid or fetal nucleic acid (i.e., fetal fraction) ascertained for a test sample. In some embodiments, a call zone is defined by a confidence level or confidence interval (e.g., a confidence interval for 95% level of confidence). A call zone sometimes is defined by a confidence level, or confidence interval based on a particular confidence level, of about 90% or greater (e.g., about 91, 92, 93, 94, 95, 96, 97, 98, 99, 99.1, 99.2, 99.3, 99.4, 99.5, 99.6, 99.7, 99.8, 99.9% or greater). In some embodiments, a call is made using a call zone and additional data or information. In some embodiments, a call is made without using a call zone. In some embodiments, a call is made based on a comparison without the use of a call zone. In some embodiments, a call is made based on visual inspection of a profile (e.g., visual inspection of read densities).

In some embodiments, a classification or call is not provided for a test sample when a test value or profile is in a no-call zone. In some embodiments, a no-call zone is defined by a value (e.g., collection of values) or profile that indicates low accuracy, high risk, high error, low level of confidence, high measure of uncertainty, the like or combination thereof. In some embodiments, a no-call zone is defined, in part, by a minority nucleic acid species quantification (e.g., a minority nucleic acid species of about 10% or less (e.g., about 9%, 8%, 7%, 6%, 5%, 4%, 3%, 2%, 1.5%, 1% or less minority nucleic acid species)). An outcome and/or classification generated for determining presence or absence of genomic instability, a genotype, phenotype, genetic variation and/or medical condition sometimes includes a null result. A null result sometimes is a data point between two clusters; sometimes is a numerical value with a standard deviation that encompasses values for both the presence and absence of genomic instability, a genotype, phenotype, genetic variation and/or medical condition; and sometimes is a data set with a profile plot that is not similar to profile plots for subjects having or free from genomic instability, or a genotype, phenotype, genetic variation or medical condition under investigation. In some embodiments, an outcome and/or classification indicative of a null result is considered a determinative result, and the determination can include a conclusion of the need for additional information and/or a repeat of data generation and/or analysis for determining the presence or absence of genomic instability, a genotype, phenotype, genetic variation and/or medical condition.

In some instances, four types of classifications generated in a classification process: true positive, false positive, true negative and false negative. The term “true positive” as used herein refers to presence of genomic instability, a genotype, phenotype, genetic variation, or medical condition correctly determined for a test sample. The term “false positive” as used herein refers to presence of genomic instability, a genotype, phenotype, genetic variation, or medical condition incorrectly determined for a test sample. The term “true negative” as used herein refers to absence of genomic instability, a genotype, phenotype, genetic variation, or medical condition correctly determined for a test sample. The term “false negative” as used herein refers to absence of genomic instability, a genotype, phenotype, genetic variation, or medical condition incorrectly determined for a test sample. Two measures of performance for a classification process can be calculated based on the ratios of these occurrences: (i) a sensitivity value, which generally is the fraction of predicted positives that are correctly identified as being positives; and (ii) a specificity value, which generally is the fraction of predicted negatives correctly identified as being negative.

### Genetic Variations/Genetic Alterations and Medical Conditions

The presence or absence of a genetic variation can be determined using a method or apparatus described herein. A genetic variation also may be referred to as a genetic alteration, and the terms are often used interchangeably herein and in the art. In certain instances, “genetic alteration” may be used to describe a somatic alteration whereby the genome in a subset of cells in a subject contains the alteration (such as, for example, in tumor or cancer cells). In certain instances, “genetic variation” may be used to describe a variation inherited from one or both parents (such as, for example, a genetic variation in a fetus).

In certain embodiments, the presence or absence of one or more genetic variations or genetic alterations is determined according to an outcome provided by methods and apparatuses described herein. A genetic variation generally is a particular genetic phenotype present in certain individuals, and often a genetic variation is present in a statistically significant subpopulation of individuals. In some embodiments, a genetic variation or genetic alteration is a chromosome abnormality or copy number alteration (e.g., aneuploidy, duplication of one or more chromosomes, loss of one or more chromosomes, partial chromosome abnormality or mosaicism (e.g., loss or gain of one or more regions of a chromosome), translocation, inversion, each of which is described in greater detail herein). Non-limiting examples of genetic variations/genetic alterations include one or more copy number alterations/variations, deletions (e.g., microdeletions), duplications (e.g., microduplications), insertions, mutations (e.g., single nucleotide variations, single nucleotide alterations), polymorphisms (e.g., single-nucleotide polymorphisms), fusions, repeats (e.g., short tandem repeats), distinct methylation sites, distinct methylation patterns, the like and combinations thereof. An insertion, repeat, deletion, duplication, mutation or polymorphism can be of any length, and in some embodiments, is about 1 base or base pair (bp) to about 250 megabases (Mb) in length. In some embodiments, an insertion, repeat, deletion, duplication, mutation or polymorphism is about 1 base or base pair (bp) to about 50,000 kilobases (kb) in length (e.g., about 10 bp, 50 bp, 100 bp, 500 bp, 1 kb, 5 kb, 10 kb, 50 kb, 100 kb, 500 kb, 1000 kb, 5000 kb or 10,000 kb in length).

A genetic variation or genetic alteration is sometime a deletion. In certain instances, a deletion is a mutation (e.g., a genetic aberration) in which a part of a chromosome or a sequence of DNA is missing. A deletion is often the loss of genetic material. Any number of nucleotides can be deleted. A deletion can comprise the deletion of one or more entire chromosomes, a region of a chromosome, an allele, a gene, an intron, an exon, any non-coding region, any coding region, a part thereof or combination thereof. A deletion can comprise a microdeletion. A deletion can comprise the deletion of a single base.

A genetic variation or genetic alteration is sometimes a duplication. In certain instances, a duplication is a mutation (e.g., a genetic aberration) in which a part of a chromosome or a sequence of DNA is copied and inserted back into the genome. In certain embodiments, a genetic duplication (e.g., duplication) is any duplication of a region of DNA. In some embodiments, a duplication is a nucleic acid sequence that is repeated, often in tandem, within a genome or chromosome. In some embodiments, a duplication can comprise a copy of one or more entire chromosomes, a region of a chromosome, an allele, a gene, an intron, an exon, any non-coding region, any coding region, part thereof or combination thereof. A duplication can comprise a microduplication. A duplication sometimes comprises one or more copies of a duplicated nucleic acid. A duplication sometimes is characterized as a genetic region repeated one or more times (e.g., repeated 1, 2, 3, 4, 5, 6, 7, 8, 9 or 10 times). Duplications can range from small regions (thousands of base pairs) to whole chromosomes in some instances. Duplications frequently occur as the result of an error in homologous recombination or due to a retrotransposon event. Duplications have been associated with certain types of proliferative diseases. Duplications can be characterized using genomic microarrays or comparative genetic hybridization (CGH).

A genetic variation or genetic alteration is sometimes an insertion. An insertion is sometimes the addition of one or more nucleotide base pairs into a nucleic acid sequence. An insertion is sometimes a microinsertion. In certain embodiments, an insertion comprises the addition of a region of a chromosome into a genome, chromosome, or part thereof. In certain embodiments, an insertion comprises the addition of an allele, a gene, an intron, an exon, any non-coding region, any coding region, part thereof or combination thereof into a genome or part thereof. In certain embodiments, an insertion comprises the addition (e.g., insertion) of nucleic acid of unknown origin into a genome, chromosome, or part thereof. In certain embodiments, an insertion comprises the addition (e.g., insertion) of a single base.

As used herein a “copy number alteration” generally is a class or type of genetic variation, genetic alteration or chromosomal aberration. A copy number alteration also may be referred to as a copy number variation, and the terms are often used interchangeably herein and in the art. In certain instances, “copy number alteration” may be used to describe a somatic alteration whereby the genome in a subset of cells in a subject contains the alteration (such as, for example, in tumor or cancer cells). In certain instances, “copy number variation” may be used to describe a variation inherited from one or both parents (such as, for example, a copy number variation in a fetus). A copy number alteration can be a deletion (e.g., microdeletion), duplication (e.g., a microduplication) or insertion (e.g., a microinsertion). Often, the prefix “micro” as used herein sometimes is a region of nucleic acid less than 5 Mb in length. A copy number alteration can include one or more deletions (e.g., microdeletion), duplications and/or insertions (e.g., a microduplication, microinsertion) of a part of a chromosome. In certain embodiments, a duplication comprises an insertion. In certain embodiments, an insertion is a duplication. In certain embodiments, an insertion is not a duplication.

In some embodiments, a copy number alteration is a copy number alteration from a tumor or cancer cell. In some embodiments, a copy number alteration is a copy number alteration from a non-cancer cell. In certain embodiments, a copy number alteration is a copy number alteration within the genome of a subject (e.g., a cancer patient) and/or within the genome of a cancer cell or tumor in a subject. A copy number alteration can be a heterozygous copy number alteration where the variation (e.g., a duplication or deletion) is present on one allele of a genome. A copy number alteration can be a homozygous copy number alteration where the alteration is present on both alleles of a genome. In some embodiments, a copy number alteration is a heterozygous or homozygous copy number alteration. In some embodiments, a copy number alteration is a heterozygous or homozygous copy number alteration from a cancer cell or non-cancer cell. A copy number alteration sometimes is present in a cancer cell genome and a non-cancer cell genome, a cancer cell genome and not a non-cancer cell genome, or a non-cancer cell genome and not a cancer cell genome.

In some embodiments, a copy number alteration is a fetal copy number alteration. Often, a fetal copy number alteration is a copy number alteration in the genome of a fetus. In some embodiments, a copy number alteration is a maternal and/or fetal copy number alteration. In certain embodiments, a maternal and/or fetal copy number alteration is a copy number alteration within the genome of a pregnant female (e.g., a female subject bearing a fetus), a female subject that gave birth or a female capable of bearing a fetus. A copy number alteration can be a heterozygous copy number alteration where the alteration (e.g., a duplication or deletion) is present on one allele of a genome. A copy number alteration can be a homozygous copy number alteration where the alteration is present on both alleles of a genome. In some embodiments, a copy number alteration is a heterozygous or homozygous fetal copy number alteration. In some embodiments, a copy number alteration is a heterozygous or homozygous maternal and/or fetal copy number alteration. A copy number alteration sometimes is present in a maternal genome and a fetal genome, a maternal genome and not a fetal genome, or a fetal genome and not a maternal genome.

“Ploidy” is a reference to the number of chromosomes present in a subject. In certain embodiments, “ploidy” is the same as “chromosome ploidy.” In humans, for example, autosomal chromosomes are often present in pairs. For example, in the absence of a genetic variation or genetic alteration, most humans have two of each autosomal chromosome (e.g., chromosomes 1-22). The presence of the normal complement of 2 autosomal chromosomes in a human is often referred to as euploid or diploid. “Microploidy” is similar in meaning to ploidy. “Microploidy” often refers to the ploidy of a part of a chromosome. The term “microploidy” sometimes is a reference to the presence or absence of a copy number alteration (e.g., a deletion, duplication and/or an insertion) within a chromosome (e.g., a homozygous or heterozygous deletion, duplication, or insertion, the like or absence thereof).

A genetic variation or genetic alteration for which the presence or absence is identified for a subject is associated with a medical condition in certain embodiments. Thus, technology described herein can be used to identify the presence or absence of one or more genetic variations or genetic alterations that are associated with a medical condition or medical state. Non-limiting examples of medical conditions include those associated with intellectual disability (e.g., Down Syndrome), aberrant cell-proliferation (e.g., cancer), presence of a micro-organism nucleic acid (e.g., virus, bacterium, fungus, yeast), and preeclampsia.

Non-limiting examples of genetic variations/genetic alterations, medical conditions and states are described hereafter.

### Chromosome Abnormalities

In some embodiments, the presence or absence of a chromosome abnormality can be determined by using a method and/or apparatus described herein. Chromosome abnormalities include, without limitation, copy number alterations, and a gain or loss of an entire chromosome or a region of a chromosome comprising one or more genes. Chromosome abnormalities include monosomies, trisomies, polysomies, loss of heterozygosity, translocations, deletions and/or duplications of one or more nucleotide sequences (e.g., one or more genes), including deletions and duplications caused by unbalanced translocations. The term “chromosomal abnormality” or “aneuploidy” as used herein refer to a deviation between the structure of the subject chromosome and a normal homologous chromosome. The term “normal” refers to the predominate karyotype or banding pattern found in healthy individuals of a particular species, for example, a euploid genome (e.g., diploid in humans, e.g., 46,XX or 46,XY). As different organisms have widely varying chromosome complements, the term “aneuploidy” does not refer to a particular number of chromosomes, but rather to the situation in which the chromosome content within a given cell or cells of an organism is abnormal. In some embodiments, the term “aneuploidy” herein refers to an imbalance of genetic material caused by a loss or gain of a whole chromosome, or part of a chromosome. An “aneuploidy” can refer to one or more deletions and/or insertions of a region of a chromosome. The term “euploid,” in some embodiments, refers a normal complement of chromosomes.

The term “monosomy” as used herein refers to lack of one chromosome of the normal complement. Partial monosomy can occur in unbalanced translocations or deletions, in which only a part of the chromosome is present in a single copy. Monosomy of sex chromosomes (45, X) causes Turner syndrome, for example. The term “disomy” refers to the presence of two copies of a chromosome. For organisms such as humans that have two copies of each chromosome (those that are diploid or “euploid”), disomy is the normal condition. For organisms that normally have three or more copies of each chromosome (those that are triploid or above), disomy is an aneuploid chromosome state. In uniparental disomy, both copies of a chromosome come from the same parent (with no contribution from the other parent).

The term “trisomy” as used herein refers to the presence of three copies, instead of two copies, of a particular chromosome. The presence of an extra chromosome 21, which is found in human Down syndrome, is referred to as “Trisomy 21.” Trisomy 18 and Trisomy 13 are two other human autosomal trisomies. Trisomy of sex chromosomes can be seen in females (e.g., 47, XXX in Triple X Syndrome) or males (e.g., 47, XXY in Klinefelter's Syndrome; or 47,XYY in Jacobs Syndrome). In some embodiments, a trisomy is a duplication of most or all of an autosome. In certain embodiments, a trisomy is a whole chromosome aneuploidy resulting in three instances (e.g., three copies) of a particular type of chromosome (e.g., instead of two instances (e.g., a pair) of a particular type of chromosome for a euploid).

The terms “tetrasomy” and “pentasomy” as used herein refer to the presence of four or five copies of a chromosome, respectively. Although rarely seen with autosomes, sex chromosome tetrasomy and pentasomy have been reported in humans, including XXXX, XXXY, XXYY, XYYY, XXXXX, XXXXY, XXXYY, XXYYY and XYYYY.

In some embodiments, the disclosure provides a method of determining whether a test sample comprises trisomies, e.g., trisomy 21, trisomy 18 or trisomy 13. The method comprises providing a set of genomic portions each coupled to a copy number alteration quantification for the test sample. The genomic portions comprise portions of a reference genome to which sequence reads obtained for sample nucleic acid from the test subject have been mapped, and the copy number alteration quantification coupled to each genomic portion has been determined from a quantification of sequence reads mapped to the genomic portion. The method further comprises filtering, by a computing device, from the set of genomic portions: (i) a first subset of portions associated with copy number alterations consistently presented in a reference set of samples, and/or (ii) a second subset of portions other than representative portions derived from groups of portions identified by a clustering process applied to a reference set of samples, thereby generating a set of filtered genomic portions not containing the first subset of portions and/or the second subset of portions. The method then transforms the set of filtered genomic portions to a reduced set of parameters by performing a principal component transformation of the set of filtered genomic portions that yields principal components for the test sample. The produced principal components can be represented in a principal component space, which contains a common principal component origin. Trisomy samples are typically grouped together and follow distinct patterns or directions, e.g., they form a vector in a two-dimensional principal component space, a plane in a three-dimensional principal component space, or a hyperplane in a n-dimension principal component space.

### Medical Disorders and Medical Conditions

Methods described herein can be applicable to any suitable medical disorder or medical condition. Non-limiting examples of medical disorders and medical conditions include cell proliferative disorders and conditions, wasting disorders and conditions, degenerative disorders and conditions, autoimmune disorders and conditions, pre-eclampsia, chemical or environmental toxicity, liver damage or disease, kidney damage or disease, vascular disease, high blood pressure, and myocardial infarction.

In some embodiments, a cell proliferative disorder or condition sometimes is a cancer, tumor, neoplasm, metastatic disease, the like or combination thereof. A cell proliferative disorder or condition sometimes is a disorder or condition of the liver, lung, spleen, pancreas, colon, skin, bladder, eye, brain, esophagus, head, neck, ovary, testes, prostate, the like or combination thereof. Non-limiting examples of cancers include hematopoietic neoplastic disorders, which are diseases involving hyperplastic/neoplastic cells of hematopoietic origin (e.g., arising from myeloid, lymphoid or erythroid lineages, or precursor cells thereof), and can arise from poorly differentiated acute leukemias (e.g., erythroblastic leukemia and acute megakaryoblastic leukemia). Certain myeloid disorders include, but are not limited to, acute promyeloid leukemia (APML), acute myelogenous leukemia (AML) and chronic myelogenous leukemia (CML). Certain lymphoid malignancies include, but are not limited to, acute lymphoblastic leukemia (ALL), which includes B-lineage ALL and T-lineage ALL, chronic lymphocytic leukemia (CLL), prolymphocytic leukemia (PLL), hairy cell leukemia (HLL) and Waldenstrom's macroglobulinemia (WM). Certain forms of malignant lymphomas include, but are not limited to, non-Hodgkin lymphoma and variants thereof, peripheral T cell lymphomas, adult T cell leukemia/lymphoma (ATL), cutaneous T-cell lymphoma (CTCL), large granular lymphocytic leukemia (LGLL), Hodgkin's disease and Reed-Sternberg disease. A cell proliferative disorder sometimes is a non-endocrine tumor or endocrine tumor. Illustrative examples of non-endocrine tumors include, but are not limited to, adenocarcinomas, acinar cell carcinomas, adenosquamous carcinomas, giant cell tumors, intraductal papillary mucinous neoplasms, mucinous cystadenocarcinomas, pancreatoblastomas, serous cystadenomas, solid and pseudopapillary tumors. An endocrine tumor sometimes is an islet cell tumor.

In some embodiments, the disclosure provides a method to determine if a test sample comprising a cancer. The method comprises providing a set of genomic portions each coupled to a copy number alteration quantification for the test sample. The genomic portions comprise portions of a reference genome to which sequence reads obtained for sample nucleic acid from the test subject have been mapped, and the copy number alteration quantification coupled to each genomic portion has been determined from a quantification of sequence reads mapped to the genomic portion. The method further comprises filtering, by a computing device, from the set of genomic portions: (i) a first subset of portions associated with copy number alterations consistently presented in a reference set of samples, and/or (ii) a second subset of portions other than representative portions derived from groups of portions identified by a clustering process applied to a reference set of samples, thereby generating a set of filtered genomic portions not containing the first subset of portions and/or the second subset of portions. The method then transforms the set of filtered genomic portions to a reduced set of parameters by performing a principal component transformation of the set of filtered genomic portions that yields principal components for the test sample. The produced principal components can be represented in a principal component space, which contains a common principal component origin. The distance between the principal components of the test sample from the common principal component origin can be determined and compared with a predetermined threshold. These tumor samples with anomalous CNA events demonstrated outlier behavior lying outside of the central cloud and thus the distance as determined above are typically greater than the predetermined threshold. In some cases, the distance is a Mahalanobis distance and the predetermined threshold greater than 300, e.g., greater than 400, greater than 450, greater than 500, or about 500 (i.e., cutoff log 10 Mahalanobis distance between 2 and 3).

In some embodiments, a wasting disorder or condition, or degenerative disorder or condition, is cirrhosis, amyotrophic lateral sclerosis (ALS), Alzheimer's disease, Parkinson's disease, multiple system atrophy, atherosclerosis, progressive supranuclear palsy, Tay-Sachs disease, diabetes, heart disease, keratoconus, inflammatory bowel disease (IBD), prostatitis, osteoarthritis, osteoporosis, rheumatoid arthritis, Huntington's disease, chronic traumatic encephalopathy, chronic obstructive pulmonary disease (COPD), tuberculosis, chronic diarrhea, acquired immune deficiency syndrome (AIDS), superior mesenteric artery syndrome, the like or combination thereof.

In some embodiments, an autoimmune disorder or condition is acute disseminated encephalomyelitis (ADEM), Addison's disease, alopecia areata, ankylosing spondylitis, antiphospholipid antibody syndrome (APS), autoimmune hemolytic anemia, autoimmune hepatitis, autoimmune inner ear disease, bullous pemphigoid, celiac disease, Chagas disease, chronic obstructive pulmonary disease, Crohns Disease (a type of idiopathic inflammatory bowel disease “IBD”), dermatomyositis, diabetes mellitus type 1, endometriosis, Goodpasture's syndrome, Graves' disease, Guillain-Barré syndrome (GBS), Hashimoto's disease, hidradenitis suppurativa, idiopathic thrombocytopenia purpura, interstitial cystitis, Lupus erythematosus, mixed connective tissue disease, morphea, multiple sclerosis (MS), myasthenia gravis, narcolepsy, euromyotonia, pemphigus vulgaris, pernicious anaemia, polymyositis, primary biliary cirrhosis, rheumatoid arthritis, schizophrenia, scleroderma, Sjögren's syndrome, temporal arteritis (also known as “giant cell arteritis”), ulcerative colitis (a type of idiopathic inflammatory bowel disease “IBD”), vasculitis, vitiligo, Wegener's granulomatosis, the like or combination thereof.

### Preeclampsia

In some embodiments, the presence or absence of preeclampsia is determined by using a method or apparatus described herein. Preeclampsia is a condition in which hypertension arises in pregnancy (e.g., pregnancy-induced hypertension) and is associated with significant amounts of protein in the urine. In certain instances, preeclampsia may be associated with elevated levels of extracellular nucleic acid and/or alterations in methylation patterns. For example, a positive correlation between extracellular fetal-derived hypermethylated RASSF1A levels and the severity of pre-eclampsia has been observed. In certain instances, increased DNA methylation is observed for the H19 gene in preeclamptic placentas compared to normal controls.

### Pathogens

In some embodiments, the presence or absence of a pathogenic condition is determined by a method or apparatus described herein. A pathogenic condition can be caused by infection of a host by a pathogen including, but not limited to, a bacterium, virus or fungus. Since pathogens typically possess nucleic acid (e.g., genomic DNA, genomic RNA, mRNA) that can be distinguishable from host nucleic acid, methods, machines and apparatus provided herein can be used to determine the presence or absence of a pathogen. Often, pathogens possess nucleic acid with characteristics unique to a particular pathogen such as, for example, epigenetic state and/or one or more sequence variations, duplications and/or deletions. Thus, methods provided herein may be used to identify a particular pathogen or pathogen variant (e.g., strain).

### Use of Cell Free Nucleic Acid

In certain instances, nucleic acid from abnormal or diseased cells associated with a particular condition or disorder is released from the cells as circulating cell-free nucleic acid (CCF-NA). For example, cancer cell nucleic acid is present in CCF-NA, and analysis of CCF-NA using methods provided herein can be used to determining whether a subject has, or is at risk of having, cancer. Analysis of the presence or absence of cancer cell nucleic acid in CCF-NA can be used for cancer screening, for example. In certain instances, levels of CCF-NA in serum can be elevated in patients with various types of cancer compared with healthy patients. Patients with metastatic diseases, for example, can sometimes have serum DNA levels approximately twice as high as non-metastatic patients. Accordingly, methods described herein can provide an outcome by processing sequencing read counts obtained from CCF-NA extracted from a sample from a subject (e.g., a subject having, suspected of having, predisposed to, or suspected as being predisposed to, a particular condition or disease).

### Markers

In certain instances, a polynucleotide in abnormal or diseased cells is modified with respect to nucleic acid in normal or non-diseased cells (e.g., single nucleotide alteration, single nucleotide variation, copy number alteration, copy number variation). In some instances, a polynucleotide is present in abnormal or diseased cells and not present in normal or non-diseased cells, and sometimes a polynucleotide is not present in abnormal or diseased cells and is present in normal or non-diseased cells. Thus, a marker sometimes is a single nucleotide alteration/variation and/or a copy number alteration/variation (e.g., a differentially expressed DNA or RNA (e.g., mRNA)). For example, patients with metastatic diseases may be identified by cancer-specific markers and/or certain single nucleotide polymorphisms or short tandem repeats, for example. Non-limiting examples of cancer types that may be positively correlated with elevated levels of circulating DNA include breast cancer, colorectal cancer, gastrointestinal cancer, hepatocellular cancer, lung cancer, melanoma, non-Hodgkin lymphoma, leukemia, multiple myeloma, bladder cancer, hepatoma, cervical cancer, esophageal cancer, pancreatic cancer, and prostate cancer. Various cancers can possess, and can sometimes release into the bloodstream, nucleic acids with characteristics that are distinguishable from nucleic acids from non-cancerous healthy cells, such as, for example, epigenetic state and/or sequence variations, duplications and/or deletions. Such characteristics can, for example, be specific to a particular type of cancer. Accordingly, methods described herein sometimes provide an outcome based on determining the presence or absence of a particular marker, and sometimes an outcome is presence or absence of a particular type of condition (e.g., a particular type of cancer or simply the presence or absence of cancer (any type)).

At block 135, the results of the analysis may be filtered using a filter subsystem. In some instances, the results of the analysis are filtered by strength and/or significance. In certain instances, the results of the analysis are filtered by strength and/or significance of the identified genomic instability. The strength and/or significance may be determined using one or more processes as described herein. For example, a filtering step can comprise the use of one or more statistical algorithms, decision analysis, comparisons, and/or the like processing steps, as described in detail herein.

In some embodiments, the results of the analysis can be filtered using any suitable number of statistical algorithms. Non-limiting examples of statistical algorithms suitable for use with filtering described herein include principal component analysis, decision trees, counternulls, multiple comparisons, omnibus test, Behrens-Fisher problem, bootstrapping, Fisher's method for combining independent tests of significance, null hypothesis, type I error, type II error, exact test, one-sample Z test, two-sample Z test, one-sample t-test, paired t-test, two-sample pooled t-test having equal variances, two-sample unpooled t-test having unequal variances, one-proportion z-test, two-proportion z-test pooled, two-proportion z-test unpooled, one-sample chi-square test, two-sample F test for equality of variances, confidence interval, credible interval, significance, meta analysis, simple linear regression, robust linear regression, the like or combinations of the foregoing.

In some embodiments, the results of the analysis can be filtered by preforming a comparison (e.g., comparing a test profile to a reference profile). Two or more data sets, two or more relationships and/or two or more profiles can be compared by a suitable method. Non-limiting examples of statistical methods suitable for comparing data sets, relationships and/or profiles include Behrens-Fisher approach, bootstrapping, Fisher's method for combining independent tests of significance, Neyman-Pearson testing, confirmatory data analysis, exploratory data analysis, exact test, F-test, Z-test, T-test, calculating and/or comparing a measure of uncertainty, a null hypothesis, counternulls and the like, a chi-square test, omnibus test, calculating and/or comparing level of significance (e.g., statistical significance), a meta analysis, a multivariate analysis, a regression, simple linear regression, robust linear regression, the like or combinations of the foregoing. In certain embodiments, comparing two or more data sets, relationships and/or profiles comprises determining and/or comparing a measure of uncertainty.

At block 140, a laboratory test report is generated for the results of the analysis and/or the filtered results of the analysis using a report subsystem. In some instances, the filtering and/or laboratory test report process includes a measure of test performance (e.g., sensitivity and/or specificity) and/or a measure of confidence (e.g., a confidence level, confidence interval). A measure of test performance and/or confidence sometimes may be obtained from a clinical validation study performed prior to performing a laboratory test for a test sample. In certain embodiments, one or more of sensitivity, specificity and/or confidence are expressed as a percentage. In some embodiments, a percentage expressed independently for each of sensitivity, specificity, or confidence level, is greater than about 90% (e.g., about 90, 91, 92, 93, 94, 95, 96, 97, 98 or 99%, or greater than 99% (e.g., about 99.5%, or greater, about 99.9% or greater, about 99.95% or greater, about 99.99% or greater)). A confidence interval expressed for a particular confidence level (e.g., a confidence level of about 90% to about 99.9% (e.g., about 95%)) can be expressed as a range of values, and sometimes is expressed as a range or sensitivities and/or specificities for a particular confidence level. Coefficient of variation (CV) in some embodiments is expressed as a percentage, and sometimes the percentage is about 10% or less (e.g., about 10, 9, 8, 7, 6, 5, 4, 3, 2 or 1%, or less than 1% (e.g., about 0.5% or less, about 0.1% or less, about 0.05% or less, about 0.01% or less)). A probability (e.g., that a particular outcome and/or classification is not due to chance) in certain embodiments is expressed as a standard score (e.g., z-score), a p-value, or result of a t-test. In some embodiments, a measured variance, confidence level, confidence interval, sensitivity, specificity and the like (e.g., referred to collectively as confidence parameters) for an outcome and/or classification can be generated using one or more data processing manipulations described herein. Specific examples of generating an outcome and/or classification and associated confidence levels are described, for example, in International Patent Application Publication Nos. WO 2013/052913, WO 2014/190286, and WO 2015/051163, the entire content of which is incorporated herein by reference for all purposes.

An outcome and/or classification for a test sample often is ordered by, and often is provided to, a health care professional or other qualified individual (e.g., physician or assistant) who transmits an outcome and/or classification to a subject from whom the test sample is obtained. In certain embodiments, an outcome and/or classification is provided using a suitable visual medium (e.g., a peripheral or component of a machine, e.g., a printer or display). A classification and/or outcome often is provided to a healthcare professional or qualified individual in the form of a report. A report typically comprises a display of an outcome and/or classification (e.g., a value, or an assessment or probability of presence or absence of genomic instability, a genotype, phenotype, genetic variation and/or medical condition), sometimes includes an associated confidence parameter, and sometimes includes a measure of performance for a test used to generate the outcome and/or classification. A report sometimes includes a recommendation for a follow-up procedure (e.g., a procedure that confirms the outcome or classification). A report sometimes includes a visual representation of a chromosome or portion thereof (e.g., a chromosome ideogram or karyogram), and sometimes shows a visualization of a duplication and/or deletion region for a chromosome (e.g., a visualization of a whole chromosome for a chromosome deletion or duplication; a visualization of a whole chromosome with a deleted region or duplicated region shown; a visualization of a portion of chromosome duplicated or deleted; a visualization of a portion of a chromosome remaining in the event of a deletion of a portion of a chromosome) identified for a test sample.

A report can be displayed in a suitable format that facilitates determination of presence or absence of genomic instability, a genotype, a phenotype, a genetic variation and/or a medical condition by a health professional or other qualified individual. Non-limiting examples of formats suitable for use for generating a report include digital data, a graph, a 2D graph, a 3D graph, and 4D graph, a picture (e.g., a jpg, bitmap (e.g., bmp), pdf, tiff, gif, raw, png, the like or suitable format), a pictograph, a chart, a table, a bar graph, a pie graph, a diagram, a flow chart, a scatter plot, a map, a histogram, a density chart, a function graph, a circuit diagram, a block diagram, a bubble map, a constellation diagram, a contour diagram, a cartogram, spider chart, Venn diagram, nomogram, and the like, or combination of the foregoing.

A report may be generated by a computer and/or by human data entry, and can be transmitted and communicated using a suitable electronic medium (e.g., via the internet, via computer, via facsimile, from one network location to another location at the same or different physical sites), or by another method of sending or receiving data (e.g., mail service, courier service and the like). Non-limiting examples of communication media for transmitting a report include auditory file, computer readable file (e.g., pdf file), paper file, laboratory file, medical record file, or any other medium described in the previous paragraph. A laboratory file or medical record file may be in tangible form or electronic form (e.g., computer readable form), in certain embodiments. After a report is generated and transmitted, a report can be received by obtaining, via a suitable communication medium, a written and/or graphical representation comprising an outcome and/or classification, which upon review allows a healthcare professional or other qualified individual to make a determination as to presence or absence of genomic instability, a genotype, phenotype, genetic variation and/or medical condition for a test sample.

An outcome and/or classification may be provided by and obtained from a laboratory (e.g., obtained from a laboratory file). A laboratory file can be generated by a laboratory that carries out one or more tests for determining presence or absence of genomic instability, a genotype, a phenotype, a genetic variation and/or a medical condition for a test sample. Laboratory personnel (e.g., a laboratory manager) can analyze information associated with test samples (e.g., test profiles, reference profiles, test values, reference values, level of deviation, patient information) underlying an outcome and/or classification. For calls pertaining to presence or absence of genomic instability, a genotype, a phenotype, a genetic variation and/or a medical condition that are close or questionable, laboratory personnel can re-run the same procedure using the same (e.g., aliquot of the same sample) or different test sample from a test subject. A laboratory may be in the same location or different location (e.g., in another country) as personnel assessing presence or absence of genomic instability, phenotype, genetic variation and/or medical condition from the laboratory file. For example, a laboratory file can be generated in one location and transmitted to another location in which the information for a test sample therein is assessed by a healthcare professional or other qualified individual, and optionally, transmitted to the subject from which the test sample was obtained. A laboratory sometimes generates and/or transmits a laboratory report containing a classification of presence or absence of genomic instability, a genotype, phenotype, a genetic variation and/or a medical condition for a test sample. A laboratory generating a laboratory test report sometimes is a certified laboratory, and sometimes is a laboratory certified under the Clinical Laboratory Improvement Amendments (CLIA).

An outcome and/or classification sometimes is a component of a diagnosis for a subject, and sometimes an outcome and/or classification is utilized and/or assessed as part of providing a diagnosis for a test sample. For example, a healthcare professional or other qualified individual may analyze an outcome and/or classification and provide a diagnosis based on, or based in part on, the outcome and/or classification. In some embodiments, determination, detection or diagnosis of a medical condition, disease, syndrome or abnormality comprises use of an outcome and/or classification determinative of presence or absence of genomic instability, a genotype, a phenotype, a genetic variation and/or a medical condition. In some embodiments, an outcome and/or classification based on counted mapped sequence reads, normalized counts and/or transformations thereof is determinative of presence or absence of genomic instability, a genotype and/or a genetic variation. In certain embodiments, a diagnosis comprises a determination of presence or absence of a condition, syndrome or abnormality. In certain instances, a diagnosis comprises a determination of a genetic variation as the nature and/or cause of genomic instability and/or a medical condition, disease, syndrome or abnormality. Thus, provided herein are methods for diagnosing presence or absence of genomic instability, a genotype, phenotype, a genetic variation and/or a medical condition for a test sample according to an outcome or classification generated by methods described herein, and optionally according to generating and transmitting a laboratory report that includes a classification for presence or absence of genomic instability, a genotype, phenotype, a genetic variation and/or a medical condition for the test sample.

An outcome and/or classification sometimes is a component of health care and/or treatment of a subject. An outcome and/or classification sometimes is utilized and/or assessed as part of providing a treatment for a subject from whom a test sample was obtained. For example, an outcome and/or classification indicative of presence or absence of genomic instability, a genotype, a phenotype, a genetic variation, and/or a medical condition is a component of health care and/or treatment of a subject from whom a test sample was obtained. Medical care, treatment and or diagnosis can be in any suitable area of health, such as medical treatment of subjects for prenatal care, cell proliferative conditions, cancer and the like, for example. An outcome and/or classification determinative of presence or absence of genomic instability, a genotype, a phenotype, a genetic variation, and/or a medical condition, disease, syndrome or abnormality by methods described herein sometimes is independently verified by further testing. Any suitable type of further test to verify an outcome and/or classification can be utilized, non-limiting examples of which include blood level test (e.g., serum test), biopsy, scan (e.g., CT scan, MRI scan), invasive sampling (e.g., amniocentesis or chorionic villus sampling), karyotyping, microarray assay, ultrasound, sonogram, and the like, for example.

A healthcare professional or qualified individual can provide a suitable healthcare recommendation based on the outcome and/or classification provided in a laboratory report. In some embodiments, a recommendation is dependent on the outcome and/or classification provided (e.g., cancer, stage and/or type of cancer, Down's syndrome, Turner syndrome, medical conditions associated with genetic variations in T13, medical conditions associated with genetic variations in T18). Non-limiting examples of recommendations that can be provided based on an outcome or classification in a laboratory report includes, without limitation, surgery, radiation therapy, chemotherapy, genetic counseling, after-birth treatment solutions (e.g., life planning, long term assisted care, medicaments, symptomatic treatments), pregnancy termination, organ transplant, blood transfusion, further testing described in the previous paragraph, the like or combinations of the foregoing. Thus, methods for treating a subject and methods for providing health care to a subject sometimes include generating a classification for presence or absence of genomic instability, a genotype, phenotype, a genetic variation and/or a medical condition for a test sample by a method described herein, and optionally generating and transmitting a laboratory report that includes a classification of presence or absence of genomic instability, a genotype, phenotype, a genetic variation and/or a medical condition for the test sample.

Generating an outcome and/or classification can be viewed as a transformation of nucleic acid sequence reads from a test sample into a representation ′f a subject's cellular nucleic acid. For example, transmuting sequence reads of nucleic acid from a subject by a method described herein, and generating an outcome and/or classification can be viewed as a transformation of relatively small sequence read fragments to a representation of relatively large and complex structure of nucleic acid in the subject. In some embodiments, an outcome and/or classification results from a transformation of sequence reads from a subject into a representation of an existing nucleic acid structure present in the subject (e.g., a genome, a chromosome, chromosome segment, mixture of circulating cell-free nucleic acid fragments in the subject).

In some embodiments, a method herein comprises treating a subject when the presence of a genetic alteration or genetic variation is determined for a test sample from the subject. In some embodiments, treating a subject comprises performing a medical procedure when the presence of a genetic alteration or genetic variation is determined for a test sample. In some embodiments, a medical procedure includes an invasive diagnostic procedure such as, for example, amniocentesis, chorionic villus sampling, biopsy, and the like. For example, a medical procedure comprising amniocentesis or chorionic villus sampling may be performed when the presence of a fetal aneuploidy is determined for a test sample from a pregnant female. In another example, a medical procedure comprising a biopsy may be performed when presence of a genetic alteration indicative of or associated with the presence of cancer is determined for a test sample from a subject. An invasive diagnostic procedure may be performed to confirm a determination of the presence of a genetic alteration or genetic variation and/or may be performed to further characterize a medical condition associated with a genetic alteration or genetic variation, for example. In some embodiments, a medical procedure may be performed as a treatment of a medical condition associated with a genetic alteration or genetic variation. Treatments may include one or more of surgery, radiation therapy, chemotherapy, pregnancy termination, organ transplant, cell transplant, blood transfusion, medicaments, symptomatic treatments, and the like, for example.

In some embodiments, a method herein comprises treating a subject when the absence of a genetic alteration or genetic variation is determined for a test sample from the subject. In some embodiments, treating a subject comprises performing a medical procedure when the absence of a genetic alteration or genetic variation is determined for a test sample. For example, when the absence of a genetic alteration or genetic variation is determined for a test sample, a medical procedure may include health monitoring, retesting, further screening, follow-up examinations, and the like. In some embodiments, a method herein comprises treating a subject consistent with a euploid pregnancy or normal pregnancy when the absence of a fetal aneuploidy, genetic variation or genetic alteration is determined for a test sample from a pregnant female. For example, a medical procedure consistent with a euploid pregnancy or normal pregnancy may be performed when the absence of a fetal aneuploidy, genetic variation or genetic alteration is determined for a test sample from a pregnant female. A medical procedure consistent with a euploid pregnancy or normal pregnancy may include one or more procedures performed as part of monitoring health of the fetus and/or the mother, or monitoring feto-maternal well-being. A medical procedure consistent with a euploid pregnancy or normal pregnancy may include one or more procedures for treating symptoms of pregnancy which may include, for example, one or more of nausea, fatigue, breast tenderness, frequent urination, back pain, abdominal pain, leg cramps, constipation, heartburn, shortness of breath, hemorrhoids, urinary incontinence, varicose veins and sleeping problems. A medical procedure consistent with a euploid pregnancy or normal pregnancy may include one or more procedures performed throughout the course of prenatal care for assessing potential risks, treating complications, addressing preexisting medical conditions (e.g., hypertension, diabetes), and monitoring the growth and development of the fetus, for example. Medical procedures consistent with a euploid pregnancy or normal pregnancy may include, for example, complete blood count (CBC) monitoring, Rh antibody testing, urinalysis, urine culture monitoring, rubella screening, hepatitis B and hepatitis C screening, sexually transmitted infection (STI) screening (e.g., screening for syphilis, chlamydia, gonorrhea), human immunodeficiency virus (HIV) screening, tuberculosis (TB) screening, alpha-fetoprotein screening, fetal heart rate monitoring (e.g., using an ultrasound transducer), uterine activity monitoring (e.g., using toco transducer), genetic screening and/or diagnostic testing for genetic disorders (e.g., cystic fibrosis, sickle cell anemia, hemophilia A), glucose screening, glucose tolerance testing, treatment of gestational diabetes, treatment of prenatal hypertension, treatment of preeclampsia, group B streptococci (GBS) blood type screening, group B strep culture, treatment of group B strep (e.g., with antibiotics), ultrasound monitoring (e.g., routine ultrasound monitoring, level II ultrasound monitoring, targeted ultrasound monitoring), non-stress test monitoring, biophysical profile monitoring, amniotic fluid index monitoring, serum testing (e.g., plasma protein-A (PAPP-A), alpha-fetoprotein (AFP), human chorionic gonadotropin (hCG), unconjugated estriol (uE3), and inhibin-A (inhA) testing), genetic testing, amniocentesis diagnostic testing and chorionic villus sampling (CVS) diagnostic testing.

In some embodiments, a method herein comprises treating a subject consistent with having no cancer when the absence of a genetic variation or genetic alteration is determined for a test sample from a subject. In certain embodiments, a medical procedure consistent with a healthy prognosis may be performed when absence of a genetic alteration or genetic variation associated with cancer is determined for a test sample. For example, medical procedures consistent with a healthy prognosis include without limitation monitoring health of the subject from whom a test sample was tested, performing a secondary test (e.g., a secondary screening test), performing a confirmatory test, monitoring one or more biomarkers associated with cancer (e.g., prostate specific antigen (PSA) in males), monitoring blood cells (e.g., red blood cells, white blood cells, platelets), monitoring one or more vital signs (e.g., heart rate, blood pressure), and/or monitoring one or more blood metabolites (e.g., total cholesterol, HDL (high-density lipoprotein), LDL (low-density lipo-protein), triglycerides, total cholesterol/HDL ratio, glucose, fibrinogen, hemoglobin, dehydroepiandrosterone (DHEA), homocysteine, C-reactive protein, hormones (e.g., thyroid stimulating hormone, testosterone, estrogen, estradiol), creatine, salt (e.g., potassium, calcium), and the like). In some embodiments, a method herein comprises performing no medical procedure, and sometimes no medical procedure that includes invasive sampling, when the absence of a genetic alteration or genetic variation is determined for a test sample.

### III. WGS PAN-CANCER SCREENING

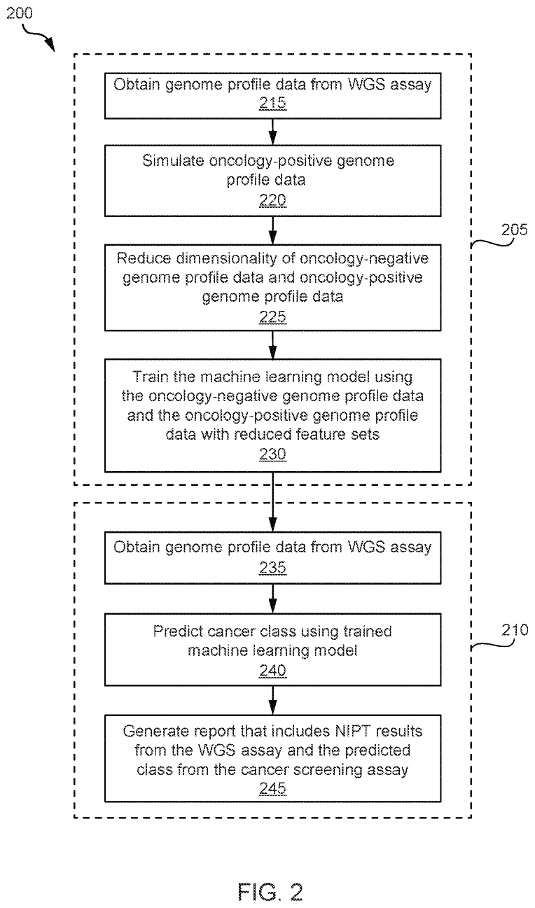

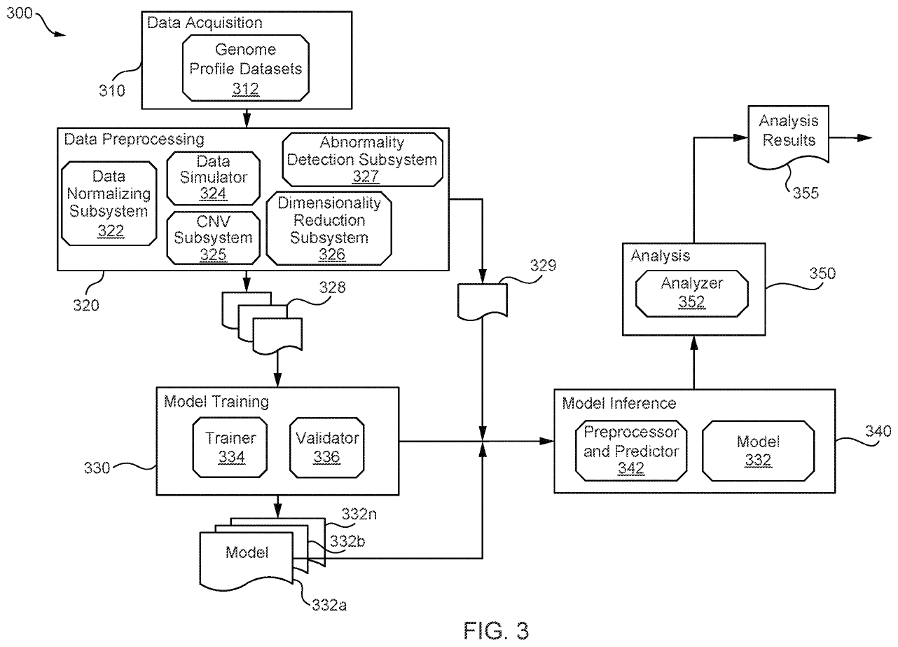

FIG. 2 shows an overview of a pan-cancer screening approach 200 that takes processed sequence read data (e.g., WGS sequence read data) from the clinical testing pipeline 100 and implements a pan-cancer screening procedure or assay for predicting whether a subject does or does not have cancer. The pan-cancer screening approach 200 generates and uses machine learning models for the predicting in two phases: a training phase 205 and an inference phase 210. The training phase 205 comprises step 215 where genome profile data is collected for multiple subjects from a WGS assay such as MaterniT® GENOME test. In some instances, the genome profile data is normalized using LOESS and principal component analysis, as described in detail herein with respect to FIG. 3. However, genome profile data collected from oncology subjects using a WGS assay is not representative of a typical pregnant subject's background DNA variability. Moreover, genome profile data collected from NIPT subjects using a WGS assay typically yields only a handful of oncology-positive samples (confirmed oncology in pregnant subject)—this being the preferable source of data but very scarce and not sufficient for training a machine learning model. Therefore, there is a challenge in acquiring oncology-positive training data such that the machine learning model training is not biased towards an oncology-negative class. To overcome this challenge, at step 220 synthetic oncology-positive genome profile data is simulated from the oncology-negative genome profile data, as described in detail herein with respect to FIG. 3.

Another challenge is that a training data set comprised of oncology-negative training data (genome profile data collected from NIPT subjects processed through a WGS assay) and oncology-positive training data (genome profile data synthetically generated from the oncology-negative training data) has a high number of variables or features (about 50,000 or more features=high dimensionality), which needs to be simplified in order to better fit a classification machine learning model. To overcome this challenge, at step 225, the training data set is pre-processed to reduce its dimensionality using one or both of the following approaches: onco-gene feature selection and PCA feature transformation, as described in detail herein with respect to FIG. 3. At step 230, the pre-processed training data set comprised of oncology-negative training data and oncology-positive training data with reduced features sets are used to train a machine learning model. In some instances, an indicator of systematic abnormality (e.g., GIN) is calculated for each example of the training data set and used as an additional feature to train the machine learning model.

At step 235, genome profile data is collected for one or more subjects from a WGS assay such as MaterniT® GENOME test. In some instances, the genome profile data is preprocessed including standardization and/or normalization. At step 240, the genome profile data for each subject is input into the machine learning model trained in step 230, and a prediction of the cancer class (e.g., oncology-positive or oncology-negative) of each subject is made by the machine learning model using the genome profile data from the WGS assay, as described in detail herein with respect to FIG. 3. In some instances, an indicator of systematic abnormality (e.g., GIN) is calculated for the genome profile data and used as an additional feature for prediction of the cancer class. Testing of the machine learning model has demonstrated 82% sensitivity on test set and 80% on extra test set with 99% specificity. Consequently, approaches described herein have been demonstrated to improve upon the sensitivity of both conventional expert analysis and statistical measures of abnormality. At step 245, a report is generated for each subject that includes both the NIPT results of the WGS assay and the predicted cancer class of the subject from the cancer screening assay.

FIG. 3 is a block diagram illustrating a system 300 for preprocessing genome profile datasets for subjects, using the preprocessed genome profile datasets to train a machine learning model, and using the trained machine learning model to predict a cancer class for a subject from genome profile data, in accordance with various embodiments. In various instances, the techniques and functionalities described in this disclosure may be implemented by a system (optionally within a distributed environment) using a set of integrated subsystems that input, output, process, and store data and information. As shown in FIG. 3, the system 300 in this example includes several subsystems: a data acquisition subsystem 310, a data preprocessing subsystem 320, a model training subsystem 330, a model inference subsystem 340, and an analysis subsystem 350.

The data acquisition subsystem 310 is configured to obtain, access, and store genome profile datasets 312 for subjects. The genome profile datasets 312 may be generated from one or more WGS assays (a comprehensive method for analyzing entire genomes) used for NIPT such as the MaterniT® GENOME test. The genome profile datasets 312 comprise raw sequence read data from the entirety, or substantially the entirety, of a subject's genome (e.g., the raw sequence read data obtained from sequence processing subsystem 120 described with respect to FIG. 1), processed sequence read data from the entirety, or substantially the entirety, of a subject's genome (e.g., the processed sequence read data obtained from data processing subsystem 125 described with respect to FIG. 1), or any combination thereof. In some instances, the genome profile datasets 312 are acquired from a clinical laboratory or health care system (e.g., NIPT assay system, a patient record system, clinical trial testing system, and the like). In some instances, the genome profile datasets 312 are acquired from a data storage structure such as a database, a laboratory or hospital information system, or the like associated with the one or more modalities for acquiring WGS assay results for subjects. In other instances, the genome profile datasets 312 are acquired directly from an NIPT assay system or clinical trial testing system such as the DRAGEN™ Bio-IT Platform from Illumina. The data acquisition subsystem 310 is further configured to provide the genome profile datasets 312 to the data preprocessing subsystem 320.

The data preprocessing subsystem 320 includes a data standardization and normalizing subsystem 322, a data simulator 324, a copy-number variation (CNV) subsystem 325, a dimensionality reduction subsystem 326, and an abnormality detection subsystem 327. The data preprocessing subsystem 320 is configured to preprocess the genome profile datasets 312 for both the training phase and the inference phase. With respect to the training phase, the data preprocessing subsystem 320 obtains the genome profile datasets 312 and performs standardization, normalization, augmentation, abnormality detection, dimensionality reduction, or any combination thereof to generate sets of training and test data 328 for training and validating one or more machine learning models 332a-n (‘n’ represents any natural number) (which may be referred to herein individually as a model 332 or collectively as the models 332). With respect to the inference phase, the data preprocessing subsystem 320 obtains the genome profile datasets 312 and performs standardization, normalization, abnormality detection, dimensionality reduction, or any combination thereof to generate sample data 329 as input for a machine learning model 332.

The data standardization and normalizing subsystem 322 standardizes and normalizes the genome profile datasets 312. Specifically, machine sequencing of samples produces a large number of short reads deposited in a file with associated quality scores (e.g., FASTQ). These reads are typically aligned to a reference sequence, such as a reference genome, and the results are deposited in an alignment file (e.g., BAM). Variants are called and their properties relevant to the sequence (e.g., type of variant) are annotated and deposited in a variant file (e.g., variant call format (VCF)). The data in the variant file are further analyzed to determine what findings are clinically relevant and reportable to a health care provider to inform medical decision making. These file specifications have been adopted in general by the clinical laboratory community, but their inherent flexibility can result in variation among assays and laboratories with respect to how data is represented within the files. Accordingly, data standardization may be implemented to create standards and transform the data taken from different sources and/or through different processes into a consistent format that adheres to the standards (e.g., placing dissimilar features on the same scale). The data standardization may be executed using any known processes. For example, standardization may include Z-score scaling, which shifts the distribution of each attribute to have a mean of zero and a standard deviation of one (unit variance).

The data normalization is performed based on LOESS and PCA. Sequencing reads aligned to a reference genome (e.g., the Hg19 reference genome or other version of the human reference genome) may be bucketed into bins (e.g., predetermined base pair length bins such as 50 Kbp consecutive bins), and the raw read counts for each bin are computed, as described herein. In some instances, after mapping and counting, the bins with low mappability and high variability are filtered or removed (see, e.g., Jensen T J, Zwiefelhofer T, Tim R C, Dzakula Z, Kim S K, Mazloom A R, et al. High-throughput massively parallel sequencing for fetal aneuploidy detection from maternal plasma. PLOS One 2013; 8: e57381, the entire content of which is incorporated herein by reference for all purposes). Bin count normalization of bins not removed and included within the genome profile datasets may be performed in two steps. First, GC-bias is accounted for by smoothing the bin count profiles with LOESS (see, e.g., Alkan C, Kidd J M, Marques-Bonet T, Aksay G, Antonacci F, Hormozdiari F, et al. Personalized copy number and segmental duplication maps using next-generation sequencing. Nat Genet 2009; 41:1061-7, the entire content of which is incorporated herein by reference for all purposes). Principal component analysis (PCA) is then used to remove higher-order artifacts for a population-based correction (see, e.g., Price A L, Patterson N J, Plenge R M, Weinblatt M E, Shadick N A, Reich D. Principal components analysis corrects for stratification in genome-wide association studies. Nat Genet 2006; 38:904-9; and Leek J T, Storey J D. Capturing heterogeneity in gene expression studies by surrogate variable analysis. PLOS Genet 2007; 3:1724-35, the entire content each is incorporated herein by reference for all purposes). The first few principal components (PC) of the euploid sample-bin count covariance matrix represent large portion of variability of a healthy genome. To reduce attenuation of the real signal in a tested sample, this background variability is accounted for by regressing LOESS-smoothed bin counts to the first few PCs and extracting the residuals. The data normalization procedure is described in more detail in Chen Zhao, John Tynan, Mathias Ehrich, Gregory Hannum, Ron Mccullough, Juan-Sebastian Saldivar, Paul Oeth, Dirk van den Boom, Cosmin Deciu, Detection of Fetal Subchromosomal Abnormalities by Sequencing Circulating Cell-Free DNA from Maternal Plasma, Clinical Chemistry, Volume 61, Issue 4, 1 Apr. 2015, Pages 608-616, https://doi.org/10.1373/clinchem. 2014.233312, the entire content of which is incorporated herein by reference for all purposes. Herein, the normalized data is referred to as “SPCA profiles” or “SPCA bin counts”.

As described herein, because the genome profile datasets 312 are acquired from WGS assays performed for NIPT, there is a high likelihood that there are very few oncology-positive examples within the genome profile datasets 312. To make a training set complete with positive cases, the data simulator 324 generates synthetic oncology-positive examples from the SPCA profiles (i.e., normalized datasets). The original sequencing results of samples from cancer patients are not adequate for this purpose for two reasons. First, they would have been processed in a different pipeline with DNA libraries different from the current WGS/NIPT pipeline data from which the genome profile datasets 312 are comprised. Second, and probably more important, reason is that the majority of samples from cancer patients are not from pregnant subjects and thus do not represent background variability of the typical sample subjected to a NIPT screening procedure. To fill this gap, a data simulation approach was developed to take results from a CNV subsystem 325 that processed the original positive sequencing data of samples from cancer patients and augment one or more euploid SPCA profiles with the signal corresponding to the CNV results statistical distribution. In some instances, the one or more euploid SPCA profiles are generated directly from examples of data from the normalized genome profile datasets 312 or a subset thereof that are classified as negative for cancer. In other instances, the one or more euploid SPCA profiles are generated by pooling together examples of data from the normalized genome profile datasets 312 or a subset thereof that are classified as negative for cancer. The cancer classification of the examples of data from the normalized genome profile datasets 312 or a subset thereof may be provided by a separate assay (e.g., oncology WGS panel, liquid biopsy, tumor biopsy, etc.), separate model, and/or medical examination (e.g., MRI or other imaging modality).

For a given oncology-positive sample, the CNV subsystem 325 identifies segments with a signal significantly deviated from a reference profile called “events”. The magnitude of an event is expressed by the z-score thus presenting a dimensionless strength of the signal. The empirical distribution of event z-scores along with the distributions of their positional features (number of events in a single subject, their spread across autosomes, relative position within chromosome) is used to form an empirical population of genomic events characteristic of cancer patients. Event features together with z-scores are subsequently drawn from this population to augment a euploid SPCA profile. Positional features define a total number of events per SPCA profile, host chromosomes, event lengths, and their relative position. Z-scores are mapped to SPCA profile augmentations with a customized mapping function (see below). Therefore, the produced population of augmented SPCA profiles presents the same statistical distribution of CNV events as the original positive data cohort and can be used to train a robust machine learning model.

### Z-Score—Bin Count Augmentation Mapping Function

After simulating positional features for a genomic event, the task is to simulate the bin counts within the affected segment [yₛₜ . . . yₑₙ], (st, en) being its start, end genomic coordinates, corresponding to the simulated segment's z-scorez_{seg}. The affected segment bin counts are modeled as the sum of background bin counts of the euploid SPCA profile and bin count augmentations:

$$[ {y}_{st} … {y}_{en} ] = [ {y}_{st}^{b} … {y}_{en}^{b} ] + [ Δ {y}_{st} … Δ {y}_{en} ] .$$

To map a profile-agnostic z-score to this particular SPCA profile bin count augmentations [Δyₛₜ . . . Δyₑₙ], a mapping function is designed. The affected segment z-score is formulated as Equation (1):

$${z}_{seg} = \frac{{Θ}_{seg} - {MEDref}_{seg}}{{MADref}_{seg}} , \qquad ( 1 )$$

where MADref_{seg} and MEDref_{seg} are median absolute deviation and median, respectively, calculated for the segment position from the reference data frame comprised of about 700 euploid samples; θ_{seg} is the augmented segment signal formulated as Equation (2):

$${Θ}_{seg} = \frac{{S}_{seg}}{{S}_{tot}} , \qquad ( 2 )$$

where

$${S}_{tot} = {\sum }_{i = 1}^{N} {y}_{i} \approx {\sum }_{i = 1}^{N} {y}_{i}^{b}$$

is the sum of the affected profile bin counts, approximated as the sum of unaffected bin counts, and

$${S}_{seg} = {\sum }_{i = st}^{en} {y}_{i}$$

is the sum of affected segment bin counts. Since the latter is not known, it is calculated from the segment signal using Equation (3):

$${S}_{seg} = {\sum }_{i = st}^{en} {y}_{i} = {Θ}_{seg} {S}_{tot} , \qquad ( 3 )$$

that, in turn, is found from the simulated z-score using Equation (4):

$${Θ}_{seg} = {z}_{seg} {MADref}_{seg} + {MEDref}_{seg} . \qquad ( 4 )$$

Next, the mean of the segment bin count augmentations and the mean of unaffected segment bin counts are found using Equation (5):

$${\overline{y}}_{seg} = \frac{{S}_{seg}}{{len}_{seg}} , \qquad ( 5 )$$

$${\overline{{y}^{b}}}_{seg} = \frac{{\sum }_{i = st}^{en} {y}_{i}^{b}}{{len}_{seg}} ,$$

where len_{seg} is the segment length measured as the number of its bins. This is sufficient to finding the mean of segment bin count augmentations Δ̅y̅_{seg}=y̅_{seg}−y̅^̅{̅b̅}̅_{seg} The standard deviation of segment bin count augmentations is approximated as the standard deviation of segment bin counts Δσ_{seg}≈σ_{seg} and is drawn from its empirical distribution. Finally, the sought-after bin count augmentations for the given segment are sampled from the Gaussian distribution [Δyₛₜ . . . Δyₑₙ]~ [symbol] (Δ̅y̅_{seg}, Δσ_{seg}). The given set of equations (1)-(5) comprises the function mapping z-scores to genomic event bin count level.

The abnormality detection subsystem 327 calculates an indicator of the systematic abnormality in a subject's genomic composition from an SPCA profile (i.e., normalized datasets) and/or synthetically augmented SPCA profile. In some instance, a process employing the GIN as an indicator of the systematic abnormality is implemented by the abnormality detection subsystem 327. The GIN is a nonnegative, continuous value calculated as the absolute deviation of observed normalized sequencing read coverage from expected normalized read coverage summed across all autosomal segments (e.g., 50,034 segments). Observed normalized sequencing read coverage is defined for each genomic segment by an autosome-specific LOESS fit of the normalized data. The data can be represented as Equation (6):

$$GIN = {\sum }_{i = 1}^{A} \vert {fit}_{i} - {exp}_{i} \vert \qquad ( 6 )$$

where the GIN is defined as the sum across all autosomal bins, from i=1 to i=A (e.g., A=50,034), of the absolute deviation of LOESS fit of the normalized genomic representation of a sample, fitᵢ, to the expected normalized genomic representation of a sample without CNAs present, expᵢ. Increasing values of GIN were observed to be indicative of increasing deviation relative to an expected normal genomic profile (see, e.g., Jensen T J, Goodman A M, Kato S, Ellison C K, Daniels G A, Kim L, Nakashe P, McCarthy E, Mazloom A R, McLennan G, Grosu D S, Ehrich M, Kurzrock R. Genome-Wide Sequencing of Cell-Free DNA Identifies Copy-Number Alterations That Can Be Used for Monitoring Response to Immunotherapy in Cancer Patients. Mol Cancer Ther. 2019 February; 18 (2): 448-458. doi: 10.1158/1535-7163.MCT-18-0535. Epub 2018 Dec. 6. PMID: 30523049, the entire content of which is incorporated herein by reference for all purposes). An empirical threshold may be used to separate negative and positive samples. The GIN has been reported to have sensitivity for the positive samples around 50-55% with a 100% specificity rate.

In some instances, the basic method implementing the GIN may be modified to either replace the GIN with an estimate of tumor content or alternatively use the estimate of tumor content in combination with the GIN as an indicator of the systematic abnormality in a subject's genomic composition. The estimate of the tumor content may be determined using a Hidden Markov Model (HMM) implemented in an ichorCNA model (see, e.g., Adalsteinsson V A, Ha G, Freeman S S, Choudhury A D, Stover D G, Parsons H A, et al. Scalable whole-exome sequencing of cell-free DNA reveals high concordance with metastatic tumors. Nat Commun 2017; 8:1324, the entire content of which is incorporated herein by reference for all purposes). The model quantifies tumor content in cfDNA (i.e., estimates the tumor fraction in cfDNA) from low-coverage WGS data without prior knowledge of tumor mutations. Application of ichorCNA on WGS data from a variety of studies has shown true positive rate (or sensitivity) in the range of 40-60% with maintained high true negative rate (or specificity) ~99% (see, e.g., S. Manier, J. Park, M. Capelletti, et al. Whole-exome sequencing of cell-free DNA and circulating tumor cells in multiple myeloma. Nat. Commun., 9 (1) (2018), p. 1691; and Xiaoji Chen, Ching-Wei Chang, Jill M. Spoerke et al. Low-pass Whole-genome Sequencing of Circulating Cell-free DNA Demonstrates Dynamic Changes in Genomic Copy Number in a Squamous Lung Cancer Clinical Cohort. Clinical Cancer Research (2019). DOI: 10.1158/1078-0432.CCR-18-1593, the entire contents of which are incorporated herein by reference for all purposes).

The oncology screening problem addressed herein can be thought of as a binary classification problem. The input data for classification is presented in a form of bin count profiles, where normalized read counts in each bin (e.g., n≈50,000 bins) are the model attributes and the labels (e.g., negative and positive) are the response variable. The GIN method described herein utilizes integral characteristics of the model attributes but does not by itself take into account the effect of individual model attributes on the response variable. To fill this gap, a supervised machine learning approach is used for building a machine learning model in the multi-dimensional space of attributes. The machine learning model is trained to take into consideration the GIN and/or tumor fraction as an indicator of the systematic abnormality in a patient genomic composition in conjunction with the effect of individual attributes on the response variable to predict whether a subject does or does not have cancer.

However, given a disparity between the training data size m (e.g., around 2500 samples) and the feature space size n being two orders of magnitude larger, the binary classification problem is ill-defined because of the data dimensionality. To address this challenge and others, the supervised machine learning approach is partially implemented by the dimensionality reduction subsystem 326 using one or both of the following approaches to reduce the data dimensionality: onco-gene feature selection and PCA feature transformation.

### Onco-Gene Feature Selection.

A hypothesis is made that variations in bin counts at the genomic regions associated with known cancers will be especially indicative of the oncology-induced mutations. Therefore, a subset b of the bins (e.g., b≈230) are selected to overlap with a predetermined number (e.g., 95) of onco-genes and SPCA-normalized counts are used at those bins for the new model attributes. With b<m the classification problem becomes well-defined and can be approached from a standard supervised machine learning standpoint. The onco-gene transformed features may be used as input information to one or more of the following algorithms: logistic regression (LR), support vector machines (SVM), and the random forest (RF) for training the machine learning model.

### PCA Feature Transformation

Another approach for reducing the data dimensionality is to map the original data onto a low-dimensional orthogonal space. PCA being well-suited for this task finds new dimensions (principal components) in the original data that best explain variability and co-variability between original attributes. The original data being mapped on first k principal components (k<<n), thus reducing the feature space n significantly and rendering the classification problem well-defined (since k<m). The PCA concept used in this approach is the same as in the data normalization step applied to the WGS data described herein but the methodology is substantially different. When reduced dimensionality is performed on the WGS data in block 205, the principal components “removed” from the data represent variability coming from healthy samples, and the data dimensionality remained the same. In contrast, PCA feature transformation performs PCA on the data comprised of both healthy and oncology samples to learn meaningful features discriminating between negative and positive classes thus reducing data redundancy and dimensionality. The PCA transformed features may be used as input information to one or more algorithms such as logistic regression (LR), support vector machines (SVM), and the random forest (RF) for training the machine learning model. To further improve the model discrimination skill, GIN may be used as an extra feature along with the oncogene transformed features and/or the PCA transformed features.

The model training subsystem 330 includes a trainer 334 and a validator 336. In the model training subsystem 330, models 332 are trained using sets of training and test data 328 provided by data preprocessing subsystem 320. The model training subsystem 330 trains models 332 to be used by the other subsystems for a given task or endpoint. A model 332 can be a machine learning model, such as (i) a convolutional neural network (“CNN”), e.g., an inception neural network, a residual neural network (“Resnet”), a U-Net, a V-Net, a single shot multibox detector (“SSD”) network, or a recurrent neural network (“RNN”), e.g., long short-term memory (“LSTM”) models or gated recurrent units (“GRUs”) models, or any combination thereof, (ii) a linear model, (iii) a regression model, (iv) a support vector machine, (v) a naïve Bayes model, (vi) a random forest model, or (vii) a decision tree model. The model can also be any other suitable machine learning model trained for a given task or endpoint, such as a three-dimensional CNN (“3DCNN”), a dynamic time warping (“DTW”) technique, a hidden Markov model (“HMM”), etc., or combinations of one or more of such techniques—e.g., CNN-HMM or MCNN (Multi-Scale Convolutional Neural Network). In certain instances, model 332 is a logistic regression classifier that uses a logistic function to model the dependent variable. The dependent variable is dichotomous in nature, i.e., there could only be two possible classes (e.g.: either the subject has cancer or not). The system 300 may employ the same type of model or different types of models for a given task or endpoint.

To train a model 332 by the model training subsystem 330, the trainer 334 is comprised of three main subsystems or services: dataset preparation, feature engineering, and model training. The dataset preparation facilitates the process of loading datasets, splitting the datasets into training and validation datasets so that the system can train and test models 332, and performing basic machine learning pre-processing (e.g., standardization, normalization, tokenizing data, annotation, augmentation, etc.). In some instances, the datasets are acquired from a data storage structure such as a database, a computing system (e.g., data preprocessing subsystem 320), or the like. The splitting may be performed randomly (e.g., a 90/10% or 70/30%) or the splitting may be performed in accordance with a more complex validation technique such as K-Fold Cross-Validation, Leave-one-out Cross-Validation, Leave-one-group-out Cross-Validation, Nested Cross-Validation, or the like to minimize sampling bias and overfitting.

Annotation can be performed manually by one or more humans (annotators such as oncologists or geneticist) confirming characteristics of each dataset and providing labels to the datasets. In some instances, the datasets may be transmitted to an annotator device to be included within a training data set. Input may be provided (e.g., by a oncologists) to the annotator device using (for example) a mouse, track pad, stylus and/or keyboard that indicates (for example) the ground truth result for a given dataset. In some instances, the dataset preparation includes obtaining a first set of the training and test data 328 as training data. For example, the training data may comprise (i) a first set of outcome predictor datasets including genome profile results for subjects that tested normal or absence of cancer (e.g., NIPT oncology-negative examples), and (ii) a second set of outcome predictor datasets including genome profile results for subjects that tested abnormal or presence of cancer (e.g., the synthetic oncology-positive examples). The training data can be annotated with labels corresponding to the first set of outcome predictor datasets and second set of outcome predictor datasets as a matrix or table of values. For example, for each predictor dataset, an indication of whether a subject tested normal or abnormal (i.e., absence or presence of cancer) may be provided as ground truth information for labels. The behavior of a model can then be adapted (e.g., through back-propagation) to minimize the difference between the generated inferences and the ground truth information.

The feature engineering includes transforming the training and test data 328 (e.g., outcome predictor datasets) into feature vectors based on features of the datasets (e.g., sequence data and indicator of the systematic abnormality). A feature vector is an ordered list of numerical properties of the observed phenomena. The feature vector represents the input features to a machine learning model that makes a prediction based on those features. In some instances, a vector is a sequence of n numbers each of which is indexed by its position in the sequence. Given some number m of objects, each of which is described by an n-component vector, a set of vectors may be organized as an m×n matrix. The training and test data 328 may be translated into vectors and matrices using one or more encoding processes such as one-hot encoding, term frequency-inverse document frequency (TF-IDF), Word2Vec, FastText, and the like, which may be implemented using pre-trained embedding models. A one-hot encoding is a representation of categorical variables as binary vectors. Each integer value is represented as a binary vector that is all zero values except the index of the integer, which is marked with a 1. TF-IDF is a statistical measure used to determine the mathematical significance of words in documents. The vectorization process is similar to one hot Encoding. Alternatively, the value corresponding to the word is assigned a TF-IDF value instead of 1. The TF-IDF value is obtained by multiplying the TF and IDF values. In Word2Vec the entire dataset is scanned, and the vector creation process is performed by determining which words the target word occurs with more often. In this way, the semantic closeness of the words to each other is also revealed. The working logic of FastText algorithm is similar to Word2Vec, but the biggest difference is that it also uses N-grams of words during training. This gives the model the ability to predict different variations of words,

The model training includes selecting hyperparameters for the model 332 and using an optimization algorithm (e.g., a stochastic gradient descent algorithm or a variant thereof such as batch gradient descent or minibatch gradient descent) to find the model parameters that correspond to the best fit between predicted and actual outputs. The hyperparameters are settings that can be tuned or optimized to control the behavior of the model 332. Most models explicitly define hyperparameters that control different aspects of the models such as memory or cost of execution. However, additional hyperparameters may be defined to adapt a model to a specific scenario. For example, the hyperparameters may include the number of hidden units of a model, the learning rate of a model, the convolution kernel width, or the number of kernels for a model.

During training, error is calculated as the difference between the actual output and the predicted output. The function that is used to compute this error is known as an objective function (e.g., a loss function or a cost function). In some instances, the objective function is a cross-entropy loss function. Error is a function of internal parameters of the model, e.g., weights and bias. For accurate predictions, the error needs to be minimized. In order to minimize the error, the model parameters are incrementally updated by minimizing the objective function over the training examples. The objective function can be constructed to measure the difference between the outputs inferred using the models and the ground truth annotated to the samples using the labels. For example, for a supervised learning-based model, the goal of the training is to learn a function “h(x)” (also sometimes referred to as the hypothesis function) that maps the training input space X to the target value space Y, h: X→Y, such that h(x) is a good predictor for the corresponding value of y. Various different techniques may be used to learn this hypothesis function. In some machine learning algorithms such as a neural network, this is done using back propagation. The current error is typically propagated backwards to a previous layer, where it is used to modify the weights and bias in such a way that the error is minimized. The weights are modified using the optimization function. Optimization functions usually calculate the error gradient, i.e., the partial derivative of the objective function with respect to weights, and the weights are modified in the opposite direction of the calculated error gradient. For example, techniques such as back propagation, random feedback, Direct Feedback Alignment (DFA), Indirect Feedback Alignment (IFA), Hebbian learning, and the like are used update the model parameters in such a manner as to minimize or maximize this objective function. This cycle is repeated until the minima of the objective function is reached.

Once a set of model parameters are identified by the model training, the model 332 has been trained and the validator 336 is configured to validate the model using the validation datasets. The validation process performed by the validator 336 includes iterative operations of inputting the validating datasets into the model 332 using a validation technique such as K-Fold Cross-Validation, Leave-one-out Cross-Validation, Leave-one-group-out Cross-Validation, Nested Cross-Validation, or the like to tune the hyperparameters and ultimately find the optimal set of hyperparameters. Once the optimal set of hyperparameters are obtained, a reserved test set of data from the validating datasets are input into the model 332 to obtain output, and the output is evaluated versus ground truth values using correlation techniques such as Bland-Altman method and the Spearman's rank correlation coefficients and calculating performance metrics such as the error, accuracy, precision, recall, receiver operating characteristic curve (ROC), etc. In some instances, the obtaining, training, and validating data process in the model training subsystem 330 can be repeatedly performed (adjusted) by the trainer 334 and validator 336 until a predetermined condition is satisfied and a set of model parameters can be provided by the model training subsystem 330.

As should be understood, other training/validation mechanisms are contemplated and may be implemented within the system 300. For example, the model 332 may be trained and hyperparameters may be tuned on datasets from the subset of obtained or filtered datasets and the datasets from the subset of obtained or filtered datasets may only be used for testing and evaluating performance of the model 332. Moreover, although the training mechanisms described herein focus on training a new model 332. These training mechanisms can also be utilized to fine tune existing models trained from other datasets. For example, in some instances, a model 332 might have been pre-trained using datasets from different modalities. In those cases, the models 332 can be used for transfer learning and retrained/validated using the training and validating data.

The model training subsystem 330 outputs trained models 332 with an optimized set of model parameters and hyperparameters for use in model inference subsystem 340. The model inference subsystem 340 generates an inference phase prediction using a preprocessor and predictor 342 and the one or more trained models 332. For example, the preprocessor and predictor 342 executes processes for inputting sample data 329 for a subject (e.g., patient) into one or more trained models 332, generating, using the one or more trained models 332, a prediction based on features extracted from sample data 329, and outputting the prediction. The one or more trained models 332 generate the prediction based on features extracted from the subject's genome profile and an indicator of the systematic abnormality such as GIN. In some instances, features of the sample data 329 may be normalized prior to input into the one or more trained models 332, as described herein with respect to data preprocessing subsystem 320. The prediction may be a binary classification of cancer for a subject (e.g., a screening assay for cancer). For example, the one or more trained models 332 may output a positive class for presence of cancer or negative class for absence of cancer (in some instances the class may be output with a confidence value).

The model inference subsystem 340 outputs the prediction or classification (e.g., classification of cancer) for optional use in analysis subsystem 350. The analysis subsystem 350 comprises one or more analyzers 352 configured for performing analysis based on the prediction or classification output by the model inference subsystem 340 and outputting analysis results 355. In some instances, the one or more analyzers 352 are configured to provide a report as part of the analysis results 355 based on the prediction or classification output by the model inference subsystem 340. The report may provide information concerning the subject (e.g., the subject's genome profile and an indicator of the systematic abnormality such as GIN) and the prediction or classification output. In some instances, the report may further include the result of the NIPT procedure or assay such as MaterniT® GENOME from which the genome profile dataset 312 was obtained. In some instances, the one or more analyzers 352 are configured to provide a recommendation as part of the analysis results 355 for a follow-up clinical diagnostic laboratory test or medical procedure based on the prediction or classification output. In some instances, the one or more analyzers 352 are configured to administer or perform a clinical diagnostic laboratory test on a sample from the subject based on the prediction or classification output. In some instances, the prediction and/or analysis results 355 may be used for diagnosing and/or treating the subject. In some instances, the analysis subsystem 350 cooperates or interplays with one or more external system to perform some or all of its analysis functions.

While not explicitly shown, it will be appreciated that the system 300 may further include a developer device associated with a developer. Communications from a developer device to components of the system 300 may indicate what types of input data, measurement data, and/or laboratory test data are to be used for the models, a number and type of models to be used, hyperparameters of each model, for example, learning rate and number of hidden layers, how data requests are to be formatted, which training data is to be used (e.g., and how to gain access to the training data) and which validation technique is to be used, and/or how the controller processes are to be configured.

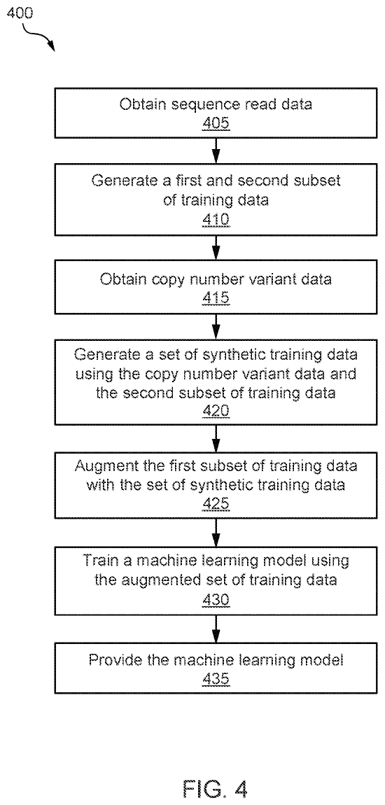

FIG. 4 is a flowchart illustrating a process 400 for preprocessing datasets and training a machine learning model according to various embodiments. The processing depicted in FIG. 4 may be implemented in software (e.g., code, instructions, program) executed by one or more processing units (e.g., processors, cores) of the respective systems, hardware, or combinations thereof (e.g., the intelligent selection machine). The software may be stored on a non-transitory store medium (e.g., on a memory device). The method presented in FIG. 4 and described below is intended to be illustrative and non-limiting. Although FIG. 4 depicts the various processing steps occurring in a particular sequence or order, this is not intended to be limiting. In certain alternative embodiments, the steps may be performed in some different orders, or some steps may also be performed in parallel. In certain embodiments, the processing or a portion of the processing depicted in FIG. 4 may be performed by systems 100 and 300 as described with respect to FIGS. 1 and 3.

At block 405, sequence read data (e.g., NIPT sequence read data) for a first group of samples are obtained (e.g., accessed). The sequence read data may be generated as part of performing a whole genome sequencing assay on samples (e.g., NIPT samples). The sequence read data includes bin count profiles comprising sequence read counts for each bin associated with a segment of a reference genome.

At block 410, a first subset of training data and a second subset of training data are generated based on the sequence read data. In some instances, each example in the first subset of training data is classified as negative for cancer. In some instances, each example in the first subset of training data is classified as negative or positive for cancer. In some instances, each example in the second subset of training data is classified as negative for cancer. The second subset of training data comprises one or more examples of sequence read data and each example in the second subset of training data may be generated directly from examples of data from the sequence read data for the first group samples that are classified as negative for cancer. Alternatively, the second subset of training data comprises one or more examples of sequence read data and each example in the second subset of training data may be generated by pooling together examples of data from the sequence read data for the first group samples that are classified as negative for cancer. The classification for the first subset of training data and the second subset of training data may be provided by a separate assay (e.g., oncology WGS panel, liquid biopsy, tumor biopsy, etc.), separate model, and/or medical examination (e.g., MRI or other imaging modality).

In some embodiments, generating the first subset of training data and the second subset of training data comprises normalizing the sequence read data by: (i) smoothing the bin count profiles using LOESS, (ii) population-based correction of the bin count profiles using principal component analysis, or (iii) both.

At block 415, copy number variant data is obtained (e.g., accessed) for a second group samples. The copy number variant data may be generated as part of performing a copy number variation assay. The copy number variant data includes information on cancer patient derived genomic events, which are copy number counts for one or more segments that deviate from a reference profile.

At block 420, a set of synthetic training data is generated based on the copy number variant data obtained from cancer patients. Generating the set of synthetic training data comprises generating an empirical population of the genomic events characteristic of the copy number variant data obtained from the cancer patients, extracting event features and standard scores from the empirical population of the genomic events, and mapping the event features and standard scores to the bin count profiles for the second subset of training data to generate the set of synthetic training data. Each example in the set of synthetic training data is classified as positive for cancer. The classification for the set of synthetic training data may be provided by a separate assay (e.g., oncology WGS panel, liquid biopsy, tumor biopsy, etc.) that generated the copy number variant data.

At block 425, the first subset of training data is augmented with the set of synthetic training data to generate an augmented set of training data. The set of synthetic training data presents the same statistical distribution of genomic events as presented in the copy number variant data. In some instances, the dimensionality of the augmented set of training data is reduced using another principal component analysis (separate from that performed at block 410 for normalizing data). The principal component analysis comprises mapping the augmented set of training data on first k principal components while discriminating between negative and positive classes, which reduces data redundancy and reduces a feature space n. In other instances, the dimensionality of the augmented set of training data is reduced using an onco-gene feature selection process. The onco-gene feature selection process comprises generating a subset of predetermined number b of bins overlapped with a predetermined number o of onco-genes and use the bin count profiles from the normalized sequence read data at the subset of predetermined number b of bins as new model attributes.

At block 430, a machine learning model is trained, using the augmented set of training data, to classify a sample as being negative for cancer or positive for cancer. The training comprises: iterative operations to find a set of parameters for the machine learning model that minimizes a loss or error function for the machine learning model. Each iteration includes finding the set of parameters for the machine learning model so that a value of the loss or error function using the set of parameters is smaller than a value of the loss or error function using another set of parameters in a previous iteration. The loss or error function is configured to measure a difference between (i) class outputs inferred for each example in the augmented set of training data, and (ii) labels that provide ground truth information for each example in the augmented set of training data, and the ground truth information identifies whether the example is classified as negative for cancer or classified as positive for cancer.

In some instances, prior to the training, an indicator of systematic abnormality is determined for each example of the augmented set of training data. The indicator is a genome instability number, a quantity of tumor content, or both. The indicator may be appended to each respective example of the augmented set of training data, and the machine learning model is trained, using the augmented set of training data with the appended indicator of systematic abnormality, to classify the sample as being negative for cancer or positive for cancer. The machine learning model uses the indicator of systematic abnormality as an additional model feature during training to find the set of parameters. In certain instances, the genome instability number is calculated as an integral of absolute deviations of a LOESS-smoothed normalized autosomal bin count profile from expectation.

At block 435, the trained machine learning model is provided (e.g., deployed in a system). In some embodiments, the performance of the trained machine learning model must meet a predetermined standard to have the trained machine learning model provided. In some embodiments, a set of model parameters is provided together with the trained machine learning model.

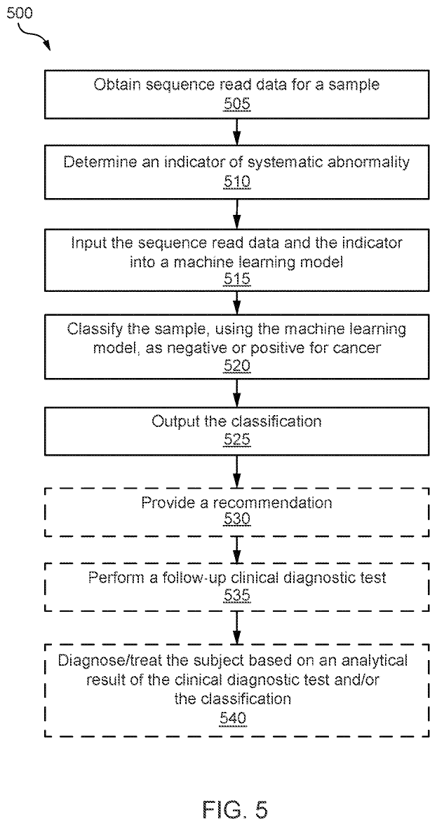

FIG. 5 is a flowchart illustrating a process 500 for using a machine learning model to predict a cancer class for a sample according to various embodiments. The processing depicted in FIG. 5 may be implemented in software (e.g., code, instructions, program) executed by one or more processing units (e.g., processors, cores) of the respective systems, hardware, or combinations thereof (e.g., the intelligent selection machine). The software may be stored on a non-transitory store medium (e.g., on a memory device). The method presented in FIG. 5 and described below is intended to be illustrative and non-limiting. Although FIG. 5 depicts the various processing steps occurring in a particular sequence or order, this is not intended to be limiting. In certain alternative embodiments, the steps may be performed in some different order, or some steps may also be performed in parallel. In certain embodiments, the processing or a portion of the processing depicted in FIG. 5 may be performed by systems 100 and 300 as described with respect to FIGS. 1 and 3.

At block 505, sequence read data (e.g., NIPT sequence read data) is obtained (e.g., accessed) for a sample (e.g., an NIPT sample). The sequence read data may be generated as part of performing a whole genome sequencing assay on a sample. The sequence read data includes a bin count profile comprising sequence read counts for each bin associated with a segment of a reference genome.

At block 510, an indicator of systematic abnormality is determined for the sample based on the sequence read data. The indicator is a genome instability number, a quantity of tumor content, or both. In certain instances, the genome instability number is calculated as an integral of absolute deviations of a LOESS-smoothed normalized autosomal bin count profile from expectation. The genome instability number may be used as an extra feature along with one or both of the data dimensionality reduction approaches: oncogene transformed features and/or the PCA transformed features described herein.

At block 515, the sequence read data and the indicator of systematic abnormality are input into a machine learning model constructed as a binary classifier. The machine learning model may be deployed locally or on an invocable end point within a cloud infrastructure, and use of the machine learning model may further comprise invoking, using the end point, the machine learning model via an application programming interface (API).

At block 520, the sample is classified, using the machine learning model, as being a negative class or positive class for cancer. The machine learning model uses the indicator of systematic abnormality as an additional model feature for predicting the negative class or the positive class. The machine learning model comprises a plurality of parameters trained using a set of augmented training data comprising: a set of original training data and a set of synthetic training data. The set of original training data comprises sequence read data for a group of samples. The sequence read data may be generated as part of performing a whole genome sequencing assay, and the sequence read data includes bin count profiles comprising sequence read counts for each bin associated with a segment of the reference genome. The set of synthetic training data may be generated based on copy number variant data. The copy number variant data may be generated as part of performing a copy number variation assay. The copy number variant data includes information on cancer patient derived genomic events, which are copy number counts for one or more segments that deviate from a reference profile.

In some instances, generating the set of synthetic training data comprises generating an empirical population of the genomic events characteristic of the copy number variant data obtained from the cancer patients, extracting event features and standard scores from the empirical population of the genomic events, and mapping the event features and standard scores to the bin count profiles for the second subset of training data to generate the set of synthetic training data. Each example in the set of synthetic training data is classified as positive for cancer.

At block 525, the negative class or the positive class for cancer is output. In some instances, the outputting comprises generating a report that includes the negative class or the positive class for cancer and a result of the whole genome sequencing assay.

At block 530, a recommendation for the one or more follow-up clinical diagnostic tests is provided based on the negative class or the positive class for cancer. In some instances, the recommendation is provided to a user such as a clinician (e.g., a health care worker associated with the subject).

At block 535, the one or more follow-up clinical diagnostic tests are performed, or performance is facilitated, on a sample from the subject to obtain an analytical result for the one or more follow-up clinical diagnostic tests.

At block 540, the subject is diagnosed and/or treated based on the analytical result for the follow-up clinical diagnostic test and/or the classification for cancer.

### IV. MACHINES, SOFTWARE AND INTERFACES

Certain processes and methods described herein (e.g., mapping, counting, normalizing, range setting, adjusting, categorizing and/or determining sequence reads, counts, levels and/or profiles) are often performed with a computer, microprocessor, software, subsystem or other machine. Methods described herein typically are computer-implemented methods, and one or more portions of a method sometimes are performed by one or more processors (e.g., microprocessors), computers, systems, apparatuses, or machines (e.g., microprocessor-controlled machine).

Computers, systems, apparatuses, machines and computer program products suitable for use often include, or are utilized in conjunction with, computer readable storage media. Non-limiting examples of computer readable storage media include memory, hard disk, CD-ROM, flash memory device and the like. Computer readable storage media generally are computer hardware, and often are non-transitory computer-readable storage media. Computer readable storage media are not computer readable transmission media, the latter of which are transmission signals per se.

Provided herein is a system comprising one or more processors and non-transitory machine readable storage medium; a set of genomic portions stored on the non-transitory machine readable medium, each of the set of genomic portions coupled to a copy number alteration quantification for a test sample. The genomic portions comprise portions of a reference genome to which sequence reads obtained for sample nucleic acid from the test subject have been mapped and a copy number alteration quantification coupled to each genomic portion has been determined from a quantification of sequence reads mapped to the genomic portion. The system further comprises program instructions to determine a presence or absence of genomic instability for the test subject according to the copy number alteration quantifications coupled to the filtered genomic portions, and the program instructions are stored on the non-transitory machine readable storage medium for execution by the one or more processors.

The system of the disclosure may further comprise program instructions to adjust the quantified sequence reads for each of the genomic portions by an adjustment process that reduces experimental bias, wherein the adjustment process generates a normalized quantification of sequence reads for each of the genomic portions wherein the program instructions are stored on the non-transitory machine readable storage medium for execution by the one or more processors.

The system may further comprise program instructions to generate a genomic instability classification for the test sample according to the normalized quantification of sequence reads for each of the genomic portions, wherein the program instructions are stored on the non-transitory machine readable storage medium for execution by the one or more processors.

Provided herein is a system comprising one or more processors and non-transitory machine readable storage medium. In some embodiments, the system comprises a set of genomic portions each coupled to a copy number alteration quantification for a test sample, wherein the genomic portions comprise portions of a reference genome to which sequence reads obtained for sample nucleic acid from the test subject have been mapped, the copy number alteration quantification coupled to each genomic portion has been determined from a quantification of sequence reads mapped to the genomic portion, and the set of genomic portions are stored on the non-transitory machine readable storage medium. The system may comprise program instructions to filter from the set of genomic portions: (i) a first subset of portions associated with copy number alterations consistently presented in a reference set of samples, and/or (ii) a second subset of portions other than representative portions derived from groups of portions identified by a clustering process applied to a reference set of samples, thereby generating a set of filtered genomic portions not containing the first subset of portions and the second subset of portions. The system may further comprise program instructions to determine a presence or absence of genomic instability for the test subject according to the copy number alteration quantifications coupled to the filtered genomic portions, wherein the program instructions are stored on the non-transitory machine readable storage medium for execution by the one or more processors.

In some embodiments, the system further comprises program instructions for transforming the set of filtered genomic portions to a reduced set of parameters, wherein the transforming comprises: performing a principal component transformation that yields principal components for the test sample, and determining a distance from a common principal component origin for the test sample. The system may also comprise program instructions for determining a distance between the principal components of the test sample from a common principal component origin.

Also provided herein is a system comprising: one or more processors and non-transitory machine readable storage medium; a set of genomic portions of a reference genome to which sequence reads obtained for sample nucleic acid from the test subject have been mapped, and for which the sequence reads mapped to the genomic portions have been quantified, wherein the set of genomic portions are stored on the non-transitory machine readable storage medium. The system may further comprise program instructions to adjust the quantified sequence reads for each of the genomic portions by an adjustment process that reduces experimental bias, wherein the adjustment process generates a normalized quantification of sequence reads for each of the genomic portions. The system may further comprise program instructions to generate a genomic instability classification for the test sample according to the normalized quantification of sequence reads for each of the genomic portions. These program instructions may be stored on the non-transitory machine readable storage medium for execution by the one or more processors. In some cases, the program instructions to reduce experimental bias is according to a GC normalized quantification of sequence reads.

Provided herein is a non-transitory machine readable storage medium comprising: a set of genomic portions each coupled to a copy number alteration quantification for a test sample. The genomic portions comprise portions of a reference genome to which sequence reads obtained for sample nucleic acid from the test subject have been mapped, and the copy number alteration quantification coupled to each genomic portion has been determined from a quantification of sequence reads mapped to the genomic portion. The non-transitory machine readable storage medium further comprises program instructions that when executed by one or more processors cause the one or more processors to perform a method comprising determining presence or absence of genomic instability for the test subject according to the copy number alteration qualifications coupled to the genomic portions.

Provided herein are computer readable storage media with an executable program stored thereon, where the program instructs a microprocessor to perform a method described herein. Provided also are computer readable storage media with an executable program subsystem stored thereon, where the program subsystem instructs a microprocessor to perform part of a method described herein. Also provided herein are systems, machines, apparatuses and computer program products that include computer readable storage media with an executable program stored thereon, where the program instructs a microprocessor to perform a method described herein. Provided also are systems, machines and apparatuses that include computer readable storage media with an executable program subsystem stored thereon, where the program subsystem instructs a microprocessor to perform part of a method described herein.

In some embodiments, the disclosure provides a non-transitory machine readable storage medium comprising program instructions that when executed by one or more processors cause the one or more processors to perform a method, the method comprising: filtering from a set of genomic portions, each of which is coupled to a copy number alteration quantification for a test sample, wherein the genomic portions comprise portions of a reference genome to which sequence reads obtained for sample nucleic acid from the test subject have been mapped, and the copy number alteration quantification coupled to each genomic portion has been determined from a quantification of sequence reads mapped to the genomic portion, (i) a first subset of portions associated with copy number alterations consistently presented in a reference set of samples, and/or (ii) a second subset of portions other than representative portions derived from groups of portions identified by a clustering process applied to a reference set of samples, thereby generating a set of filtered genomic portions not containing the first subset of portions and the second subset of portions; and determining presence or absence of genomic instability for the test subject according to the copy number alteration qualifications coupled to the filtered genomic portions.

In some embodiments, the non-transitory machine readable storage medium further comprises: program Instructions for transforming the set of filtered genomic portions to a reduced set of parameters, wherein the transforming comprises: performing a principal component transformation that yields principal components for the test sample, and determining a distance from a common principal component origin for the test sample; and determining a distance between the principal components of the test sample from a common principal component origin.

The non-transitory machine readable storage medium may further comprise program instructions that when executed by one or more processors cause the one or more processors to perform a method comprising: adjusting the quantified sequence reads for each of the genomic portions by an adjustment process that reduces experimental bias, wherein the adjustment process generates a normalized quantification of sequence reads for each of the genomic portions.

Also provided herein is a non-transitory machine readable storage medium comprising: program instructions that when executed by one or more processors cause the one or more processors to perform a method comprising: adjusting the quantified sequence reads for each of a set of genomic portions by an adjustment process that reduces experimental bias, wherein the set of genomic portions of a reference genome to which sequence reads obtained for sample nucleic acid from the test subject have been mapped, and for which the sequence reads mapped to the genomic portions have been quantified, and wherein the adjustment process generates a normalized quantification of sequence reads for each of the genomic portions; and generating a genomic instability classification for the test sample according to the normalized quantification of sequence reads for each of the genomic portions. In some embodiments, the program instructions to reduce experimental bias is according to a GC normalized quantification of sequence reads.

Thus, also provided are computer program products. A computer program product often includes a computer usable medium that includes a computer readable program code embodied therein, the computer readable program code adapted for being executed to implement a method or part of a method described herein. Computer usable media and readable program code are not transmission media (i.e., transmission signals per se). Computer readable program code often is adapted for being executed by a processor, computer, system, apparatus, or machine.

In some embodiments, methods described herein (e.g., quantifying, counting, filtering, normalizing, transforming, clustering and/or determining sequence reads, counts, levels, profiles and/or outcomes) are performed by automated methods. In some embodiments, one or more steps of a method described herein are carried out by a microprocessor and/or computer, and/or carried out in conjunction with memory. In some embodiments, an automated method is embodied in software, subsystems, microprocessors, peripherals and/or a machine comprising the like, that perform methods described herein. As used herein, software refers to computer readable program instructions that, when executed by a microprocessor, perform computer operations, as described herein.

Sequence reads, counts, levels and/or profiles sometimes are referred to as “data” or “data sets.” In some embodiments, data or data sets can be characterized by one or more features or variables (e.g., sequence based (e.g., GC content, specific nucleotide sequence, the like), function specific (e.g., expressed genes, cancer genes, the like), location based (genome specific, chromosome specific, portion or portion-specific), the like and combinations thereof). In certain embodiments, data or data sets can be organized into a matrix having two or more dimensions based on one or more features or variables. Data organized into matrices can be organized using any suitable features or variables. In certain embodiments, data sets characterized by one or more features or variables sometimes are processed after counting.

Machines, software and interfaces may be used to conduct methods described herein. Using machines, software and interfaces, a user may enter, request, query or determine options for using particular information, programs or processes (e.g., mapping sequence reads, processing mapped data and/or providing an outcome), which can involve implementing statistical analysis algorithms, statistical significance algorithms, statistical algorithms, iterative steps, validation algorithms, and graphical representations, for example. In some embodiments, a data set may be entered by a user as input information, a user may download one or more data sets by suitable hardware media (e.g., flash drive), and/or a user may send a data set from one system to another for subsequent processing and/or providing an outcome (e.g., send sequence read data from a sequencer to a computer system for sequence read mapping; send mapped sequence data to a computer system for processing and yielding an outcome and/or report).

A system typically comprises one or more machines. Each machine comprises one or more of memory, one or more microprocessors, and instructions. Where a system includes two or more machines, some or all of the machines may be located at the same location, some or all of the machines may be located at different locations, all of the machines may be located at one location and/or all of the machines may be located at different locations. Where a system includes two or more machines, some or all of the machines may be located at the same location as a user, some or all of the machines may be located at a location different than a user, all of the machines may be located at the same location as the user, and/or all of the machine may be located at one or more locations different than the user.

A system sometimes comprises a computing machine and a sequencing apparatus or machine, where the sequencing apparatus or machine is configured to receive physical nucleic acid and generate sequence reads, and the computing apparatus is configured to process the reads from the sequencing apparatus or machine. The computing machine sometimes is configured to determine a classification outcome from the sequence reads.

A user may, for example, place a query to software which then may acquire a data set via internet access, and in certain embodiments, a programmable microprocessor may be prompted to acquire a suitable data set based on given parameters. A programmable microprocessor also may prompt a user to select one or more data set options selected by the microprocessor based on given parameters. A programmable microprocessor may prompt a user to select one or more data set options selected by the microprocessor based on information found via the internet, other internal or external information, or the like. Options may be chosen for selecting one or more data feature selections, one or more statistical algorithms, one or more statistical analysis algorithms, one or more statistical significance algorithms, iterative steps, one or more validation algorithms, and one or more graphical representations of methods, machines, apparatuses, computer programs or a non-transitory computer-readable storage medium with an executable program stored thereon.

Systems addressed herein may comprise general components of computer systems, such as, for example, network servers, laptop systems, desktop systems, handheld systems, personal digital assistants, computing kiosks, and the like. A computer system may comprise one or more input means such as a keyboard, touch screen, mouse, voice recognition or other means to allow the user to enter data into the system. A system may further comprise one or more outputs, including, but not limited to, a display screen (e.g., CRT or LCD), speaker, FAX machine, printer (e.g., laser, ink jet, impact, black and white or color printer), or other output useful for providing visual, auditory and/or hardcopy output of information (e.g., outcome and/or report).

In a system, input and output components may be connected to a central processing unit which may comprise among other components, a microprocessor for executing program instructions and memory for storing program code and data. In some embodiments, processes may be implemented as a single user system located in a single geographical site. In certain embodiments, processes may be implemented as a multi-user system. In the case of a multi-user implementation, multiple central processing units may be connected by means of a network. The network may be local, encompassing a single department in one portion of a building, an entire building, span multiple buildings, span a region, span an entire country or be worldwide. The network may be private, being owned and controlled by a provider, or it may be implemented as an internet-based service where the user accesses a web page to enter and retrieve information. Accordingly, in certain embodiments, a system includes one or more machines, which may be local or remote with respect to a user. More than one machine in one location or multiple locations may be accessed by a user, and data may be mapped and/or processed in series and/or in parallel. Thus, a suitable configuration and control may be utilized for mapping and/or processing data using multiple machines, such as in local network, remote network and/or “cloud” computing platforms.

A system can include a communications interface in some embodiments. A communications interface allows for transfer of software and data between a computer system and one or more external devices. Non-limiting examples of communications interfaces include a modem, a network interface (such as an Ethernet card), a communications port, a PCMCIA slot and card, and the like. Software and data transferred via a communications interface generally are in the form of signals, which can be electronic, electromagnetic, optical and/or other signals capable of being received by a communications interface. Signals often are provided to a communications interface via a channel. A channel often carries signals and can be implemented using wire or cable, fiber optics, a phone line, a cellular phone link, an RF link and/or other communications channels. Thus, in an example, a communications interface may be used to receive signal information that can be detected by a signal detection subsystem.

Data may be input by a suitable device and/or method, including, but not limited to, manual input devices or direct data entry devices (DDEs). Non-limiting examples of manual devices include keyboards, concept keyboards, touch sensitive screens, light pens, mouse, tracker balls, joysticks, graphic tablets, scanners, digital cameras, video digitizers and voice recognition devices. Non-limiting examples of DDEs include bar code readers, magnetic strip codes, smart cards, magnetic ink character recognition, optical character recognition, optical mark recognition, and turnaround documents.

In some embodiments, output from a sequencing apparatus or machine may serve as data that can be input via an input device. In certain embodiments, mapped sequence reads may serve as data that can be input via an input device. In certain embodiments, nucleic acid fragment size (e.g., length) may serve as data that can be input via an input device. In certain embodiments, output from a nucleic acid capture process (e.g., genomic region origin data) may serve as data that can be input via an input device. In certain embodiments, a combination of nucleic acid fragment size (e.g., length) and output from a nucleic acid capture process (e.g., genomic region origin data) may serve as data that can be input via an input device. In certain embodiments, simulated data is generated by an in silico process and the simulated data serves as data that can be input via an input device. The term “in silico” refers to research and experiments performed using a computer. In silico processes include, but are not limited to, mapping sequence reads and processing mapped sequence reads according to processes described herein.

A system may include software useful for performing a process or part of a process described herein, and software can include one or more subsystems for performing such processes (e.g., sequencing subsystem, logic processing subsystem, data display organization subsystem). The term “software” refers to computer readable program instructions that, when executed by a computer, perform computer operations. Instructions executable by the one or more microprocessors sometimes are provided as executable code, that when executed, can cause one or more microprocessors to implement a method described herein.

A subsystem described herein can exist as software, and instructions (e.g., processes, routines, subroutines) embodied in the software can be implemented or performed by a microprocessor. For example, a subsystem (e.g., a software subsystem) can be a part of a program that performs a particular process or task. The term “subsystem” refers to a self-contained functional unit that can be used in a larger machine or software system. A subsystem can comprise a set of instructions for carrying out a function of the subsystem. A subsystem can transform data and/or information. Data and/or information can be in a suitable form. For example, data and/or information can be digital or analogue. In certain embodiments, data and/or information sometimes can be packets, bytes, characters, or bits. In some embodiments, data and/or information can be any gathered, assembled or usable data or information. Non-limiting examples of data and/or information include a suitable media, pictures, video, sound (e.g. frequencies, audible or non-audible), numbers, constants, a value, objects, time, functions, instructions, maps, references, sequences, reads, mapped reads, levels, ranges, thresholds, signals, displays, representations, or transformations thereof. A subsystem can accept or receive data and/or information, transform the data and/or information into a second form, and provide or transfer the second form to a machine, peripheral, component or another subsystem. A subsystem can perform one or more of the following non-limiting functions: mapping sequence reads, providing counts, assembling portions, providing or determining a level, providing a count profile, normalizing (e.g., normalizing reads, normalizing counts, and the like), providing a normalized count profile or levels of normalized counts, comparing two or more levels, providing uncertainty values, providing or determining expected levels and expected ranges (e.g., expected level ranges, threshold ranges and threshold levels), providing adjustments to levels (e.g., adjusting a first level, adjusting a second level, adjusting a profile of a chromosome or a part thereof, and/or padding), providing identification (e.g., identifying a copy number alteration, genetic variation/genetic alteration or aneuploidy), categorizing, plotting, and/or determining an outcome, for example. A microprocessor can, in certain embodiments, carry out the instructions in a subsystem. In some embodiments, one or more microprocessors are required to carry out instructions in a subsystem or group of subsystems. A subsystem can provide data and/or information to another subsystem, machine or source and can receive data and/or information from another subsystem, machine or source.

A computer program product sometimes is embodied on a tangible computer-readable medium, and sometimes is tangibly embodied on a non-transitory computer-readable medium. A subsystem sometimes is stored on a computer readable medium (e.g., disk, drive) or in memory (e.g., random access memory). A subsystem and microprocessor capable of implementing instructions from a subsystem can be located in a machine or in a different machine. A subsystem and/or microprocessor capable of implementing an instruction for a subsystem can be located in the same location as a user (e.g., local network) or in a different location from a user (e.g., remote network, cloud system). In embodiments in which a method is carried out in conjunction with two or more subsystems, the subsystems can be located in the same machine, one or more subsystems can be located in different machine in the same physical location, and one or more subsystems may be located in different machines in different physical locations.

A machine, in some embodiments, comprises at least one microprocessor for carrying out the instructions in a subsystem. Sequence read quantifications (e.g., counts) sometimes are accessed by a microprocessor that executes instructions configured to carry out a method described herein. Sequence read quantifications that are accessed by a microprocessor can be within memory of a system, and the counts can be accessed and placed into the memory of the system after they are obtained. In some embodiments, a machine includes a microprocessor (e.g., one or more microprocessors) which microprocessor can perform and/or implement one or more instructions (e.g., processes, routines and/or subroutines) from a subsystem. In some embodiments, a machine includes multiple microprocessors, such as microprocessors coordinated and working in parallel. In some embodiments, a machine operates with one or more external microprocessors (e.g., an internal or external network, server, storage device and/or storage network (e.g., a cloud)). In some embodiments, a machine comprises a subsystem (e.g., one or more subsystems). A machine comprising a subsystem often is capable of receiving and transferring one or more of data and/or information to and from other subsystems.

In certain embodiments, a machine comprises peripherals and/or components. In certain embodiments, a machine can comprise one or more peripherals or components that can transfer data and/or information to and from other subsystems, peripherals and/or components. In certain embodiments, a machine interacts with a peripheral and/or component that provides data and/or information. In certain embodiments, peripherals and components assist a machine in carrying out a function or interact directly with a subsystem. Non-limiting examples of peripherals and/or components include a suitable computer peripheral, I/O or storage method or device including but not limited to scanners, printers, displays (e.g., monitors, LED, LCT or CRTs), cameras, microphones, pads (e.g., iPads, tablets), touch screens, smart phones, mobile phones, USB I/O devices, USB mass storage devices, keyboards, a computer mouse, digital pens, modems, hard drives, jump drives, flash drives, a microprocessor, a server, CDs, DVDs, graphic cards, specialized I/O devices (e.g., sequencers, photo cells, photo multiplier tubes, optical readers, sensors, etc.), one or more flow cells, fluid handling components, network interface controllers, ROM, RAM, wireless transfer methods and devices (Bluetooth, WiFi, and the like,), the world wide web (www), the internet, a computer and/or another subsystem.

Software comprising program instructions often is provided on a program product containing program instructions recorded on a computer readable medium, including, but not limited to, magnetic media including floppy disks, hard disks, and magnetic tape; and optical media including CD-ROM discs, DVD discs, magneto-optical discs, flash memory devices (e.g., flash drives), RAM, floppy discs, the like, and other such media on which the program instructions can be recorded. In online implementation, a server and web site maintained by an organization can be configured to provide software downloads to remote users, or remote users may access a remote system maintained by an organization to remotely access software. Software may obtain or receive input information. Software may include a subsystem that specifically obtains or receives data (e.g., a data receiving subsystem that receives sequence read data and/or mapped read data) and may include a subsystem that specifically processes the data (e.g., a processing subsystem that processes received data (e.g., filters, normalizes, provides an outcome and/or report). The terms “obtaining” and “receiving” input information refers to receiving data (e.g., sequence reads, mapped reads) by computer communication means from a local, or remote site, human data entry, or any other method of receiving data. The input information may be generated in the same location at which it is received, or it may be generated in a different location and transmitted to the receiving location. In some embodiments, input information is modified before it is processed (e.g., placed into a format amenable to processing (e.g., tabulated)).

Software can include one or more algorithms in certain embodiments. An algorithm may be used for processing data and/or providing an outcome or report according to a finite sequence of instructions. An algorithm often is a list of defined instructions for completing a task. Starting from an initial state, the instructions may describe a computation that proceeds through a defined series of successive states, eventually terminating in a final ending state. The transition from one state to the next is not necessarily deterministic (e.g., some algorithms incorporate randomness). By way of example, and without limitation, an algorithm can be a search algorithm, sorting algorithm, merge algorithm, numerical algorithm, graph algorithm, string algorithm, modeling algorithm, computational genomic algorithm, combinatorial algorithm, machine learning algorithm, cryptography algorithm, data compression algorithm, parsing algorithm and the like. An algorithm can include one algorithm or two or more algorithms working in combination. An algorithm can be of any suitable complexity class and/or parameterized complexity. An algorithm can be used for calculation and/or data processing, and in some embodiments, can be used in a deterministic or probabilistic/predictive approach. An algorithm can be implemented in a computing environment by use of a suitable programming language, non-limiting examples of which are C, C++, Java, Perl, Python, Fortran, and the like. In some embodiments, an algorithm can be configured or modified to include margin of errors, statistical analysis, statistical significance, and/or comparison to other information or data sets (e.g., applicable when using a neural net or clustering algorithm).

In certain embodiments, several algorithms may be implemented for use in software. These algorithms can be trained with raw data in some embodiments. For each new raw data sample, the trained algorithms may produce a representative processed data set or outcome. A processed data set sometimes is of reduced complexity compared to the parent data set that was processed. Based on a processed set, the performance of a trained algorithm may be assessed based on sensitivity and specificity, in some embodiments. An algorithm with the highest sensitivity and/or specificity may be identified and utilized, in certain embodiments.

In certain embodiments, simulated (or simulation) data can aid data processing, for example, by training an algorithm or testing an algorithm. In some embodiments, simulated data includes hypothetical various samplings of different groupings of sequence reads. Simulated data may be based on what might be expected from a real population or may be skewed to test an algorithm and/or to assign a correct classification. Simulated data also is referred to herein as “virtual” data. Simulations can be performed by a computer program in certain embodiments. One possible step in using a simulated data set is to evaluate the confidence of identified results, e.g., how well a random sampling matches or best represents the original data. One approach is to calculate a probability value (p-value), which estimates the probability of a random sample having better score than the selected samples. In some embodiments, an empirical model may be assessed, in which it is assumed that at least one sample matches a reference sample (with or without resolved variations). In some embodiments, another distribution, such as a Poisson distribution for example, can be used to define the probability distribution.

A system may include one or more microprocessors in certain embodiments. A microprocessor can be connected to a communication bus. A computer system may include a main memory, often random access memory (RAM), and can also include a secondary memory. Memory in some embodiments comprises a non-transitory computer-readable storage medium. Secondary memory can include, for example, a hard disk drive and/or a removable storage drive, representing a floppy disk drive, a magnetic tape drive, an optical disk drive, memory card and the like. A removable storage drive often reads from and/or writes to a removable storage unit. Non-limiting examples of removable storage units include a floppy disk, magnetic tape, optical disk, and the like, which can be read by and written to by, for example, a removable storage drive. A removable storage unit can include a computer-usable storage medium having stored therein computer software and/or data.

A microprocessor may implement software in a system. In some embodiments, a microprocessor may be programmed to automatically perform a task described herein that a user could perform. Accordingly, a microprocessor, or algorithm conducted by such a microprocessor, can require little to no supervision or input from a user (e.g., software may be programmed to implement a function automatically). In some embodiments, the complexity of a process is so large that a single person or group of persons could not perform the process in a timeframe short enough for determining the presence or absence of a genetic variation or genetic alteration.

In some embodiments, secondary memory may include other similar means for allowing computer programs or other instructions to be loaded into a computer system. For example, a system can include a removable storage unit and an interface device. Non-limiting examples of such systems include a program cartridge and cartridge interface (such as that found in video game devices), a removable memory chip (such as an EPROM, or PROM) and associated socket, and other removable storage units and interfaces that allow software and data to be transferred from the removable storage unit to a computer system.

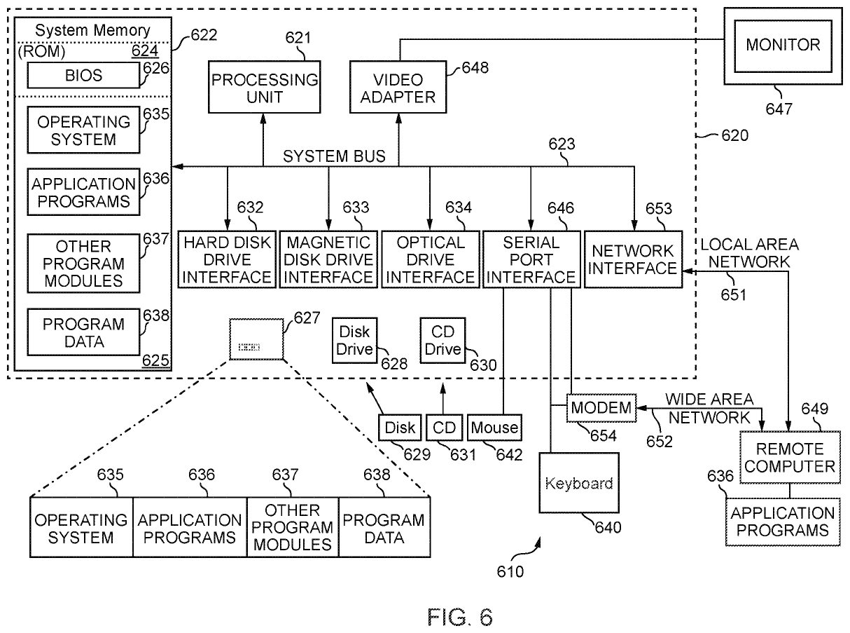

FIG. 6 illustrates a non-limiting example of a computing environment 610 in which various systems, methods, algorithms, machine learning models, and data structures described herein may be implemented. The computing environment 610 is only one example of a suitable computing environment and is not intended to suggest any limitation as to the scope of use or functionality of the systems, methods, and data structures described herein. Neither should computing environment 610 be interpreted as having any dependency or requirement relating to any one or combination of components illustrated in computing environment 610. A subset of systems, methods, and data structures shown in FIG. 6 can be utilized in certain embodiments. Systems, methods, and data structures described herein are operational with numerous other general purpose or special purpose computing system environments or configurations. Examples of known computing systems, environments, and/or configurations that may be suitable include, but are not limited to, personal computers, server computers, thin clients, thick clients, hand-held or laptop devices, multiprocessor systems, microprocessor-based systems, set top boxes, programmable consumer electronics, network PCs, minicomputers, mainframe computers, distributed computing environments that include any of the above systems or devices, and the like.

The computing environment 610 of FIG. 6 includes a general purpose computing device in the form of a computer 620, including a processing unit 621, a system memory 622, and a system bus 623 that operatively couples various system components including the system memory 622 to the processing unit 621. There may be only one or there may be more than one processing unit 621, such that the processor of computer 620 includes a single central-processing unit (CPU), or a plurality of processing units, commonly referred to as a parallel processing environment. The computer 620 may be a conventional computer, a distributed computer, or any other type of computer.

The system bus 623 may be any of several types of bus structures including a memory bus or memory controller, a peripheral bus, and a local bus using any of a variety of bus architectures. The system memory may also be referred to as simply the memory, and includes read only memory (ROM) 624 and random access memory (RAM) 625. A basic input/output system (BIOS) 626, containing the basic routines that help to transfer information between elements within the computer 620, such as during start-up, is stored in ROM 624. The computer 620 may further include a hard disk drive interface 627 for reading from and writing to a hard disk, not shown, a magnetic disk drive 628 for reading from or writing to a removable magnetic disk 629, and an optical disk drive 630 for reading from or writing to a removable optical disk 631 such as a CD ROM or other optical media.

The hard disk drive 627, magnetic disk drive 628, and optical disk drive 630 are connected to the system bus 623 by a hard disk drive interface 632, a magnetic disk drive interface 633, and an optical disk drive interface 634, respectively. The drives and their associated computer-readable media provide nonvolatile storage of computer-readable instructions, data structures, program subsystems and other data for the computer 620. Any type of computer-readable media that can store data that is accessible by a computer, such as magnetic cassettes, flash memory cards, digital video disks, Bernoulli cartridges, random access memories (RAMs), read only memories (ROMs), and the like, may be used in the operating environment.

A number of program subsystems may be stored on the hard disk drive 627, magnetic disk 629, optical disk 631, ROM 624, or RAM 625, including an operating system 635, one or more application programs 636, other program subsystems 637, and program data 638. A user may enter commands and information into the computer 620 through input devices such as a keyboard 640 and pointing device 642 (e.g., a mouse). Other input devices (not shown) may include a microphone, joystick, game pad, satellite dish, scanner, or the like. These and other input devices are often connected to the processing unit 621 through a serial port interface 646 that is coupled to the system bus, but may be connected by other interfaces, such as a parallel port, game port, or a universal serial bus (USB). A monitor 647 or other type of display device is also connected to the system bus 623 via an interface, such as a video adapter 648. In addition to the monitor, computers typically include other peripheral output devices (not shown), such as speakers and printers.

The computer 620 may operate in a networked environment using logical connections to one or more remote computers, such as remote computer 649. These logical connections may be achieved by a communication device coupled to or a part of the computer 620, or in other manners. The remote computer 649 may be another computer, a server, a router, a network PC, a client, a peer device or other common network node, and typically includes many or all of the elements described above relative to the computer 620, although only a memory storage device has been illustrated in FIG. 6. The logical connections depicted in FIG. 6 include a local-area network (LAN) 651 and a wide-area network (WAN) 652. Such networking environments are commonplace in office networks, enterprise-wide computer networks, intranets and the Internet, which all are types of networks.

When used in a LAN-networking environment, the computer 620 is connected to the local network 651 through a network interface or adapter 653, which is one type of communications device. When used in a WAN-networking environment, the computer 620 often includes a modem 654, a type of communications device, or any other type of communications device for establishing communications over the wide area network 652. The modem 654, which may be internal or external, is connected to the system bus 623 via the serial port interface 646. In a networked environment, program subsystems depicted relative to the personal computer 620, or portions thereof, may be stored in the remote memory storage device. It is appreciated that the network connections shown are non-limiting examples and other communications.

### V. EXAMPLES

The models and techniques implemented in various embodiments may be better understood by referring to the following examples.

### Example 1: Pan-Cancer Screening in Whole Genome Sequencing Non-Invasive Prenatal Testing Procedures

### Challenges

As described herein, there are various challenges of building a machine learning model to extract an oncology signal and predict presence or absence of cancer in a sample. More specifically, oncology patient data (~500 samples) processed through MaterniT® GENOME pipeline is not representative of the pregnant women background DNA variability (highly imbalanced data). Further, data processing protocols vary from that in production. Lastly, NIPT data only has a handful of positive samples (confirmed oncology positive in pregnant women)—preferable source of information but very scarce and not sufficient for training a machine learning model (positive examples are too scarce to train a robust machine learning model). Therefore, there is a need for positive training data.

### Data Simulation

Direct simulation of oncology-positive data for NIPT patients is problematic for a number of reasons: (i) not clear how to represent cancer signal, especially for samples with normal GIN (overall genome variability is within healthy range), and (ii) NIPT oncology data is scarce, and prone to reproducing DNA variability of particular individuals. Data simulation should come from information that: (i) presumably contains oncology signal, (ii) that is distilled and does not feature biological variability, and (iii) is diverse enough to represent population of various cancer types across different subjects (e.g., patients). The Chromosomal Aberration Decision Tree (CADET) is the central module of Applicant's Maternal Health products pipeline that predicts genomic CNV events for NIPT patients. The CADET results are presented in form of events as single data points, each representing a feature vector: {host chromosome, coordinates, length, Z-score, event variance}. For oncology data simulation, findings from CADET, a CNV events prediction module, can be used.

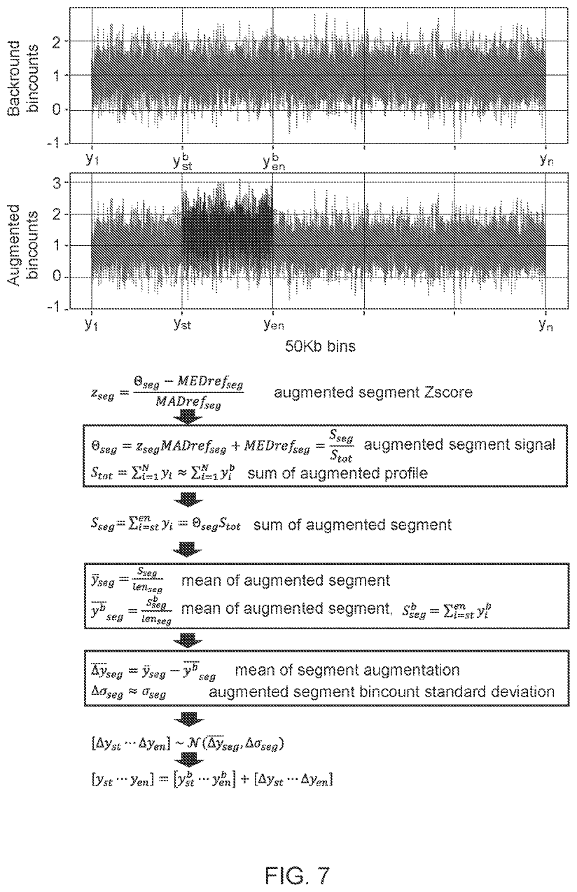

Two sources of information are combined to produce final simulated NIPT oncology bin count profiles: (i) background healthy NIPT bin count profile, and (ii) simulated genomic events. In other words, the data simulation technique combines background normal profiles with CADET CNV events representing the oncology signal. In order to achieve this, the data simulation technique was developed to map the CNV event characteristics back to the bin count level of the healthy NIPT bin count profile as follows:
Used CADET events of oncology (non-NIPT) samples to simulate oncology NIPT data at bin count level;
Used NPP samples (pooled plasma from many negative NIPT patients) as the underlying background NPP profile;
To spike-in the event signal into the background NPP profile, Z-score and sigma were mapped to the augmentations at bin level, hence mapping function was designed as described with respect to FIG. 4 and further shown in FIG. 7;
   Sampled arguments of the mapping function (Z-score, sigma) along with event features from the underlying distributions
      Event features were simulated by randomly sampling from empirical conditional distributions: ~500 (non-NIPT) oncology samples;
      The event features simulated were:
         Number of events in a sample
         Number of events on each chromosome.
         Start position of first event on a chromosome
         Length of edge events
         Length of in-between events
         Length of gaps between events
         Event Z-score
         Variance of event
   Segment augmentations were simulated using parametric (Gaussian) distribution;
      Parameters (mean, sigma) of the parametric (Gaussian) distribution were derived analytically from data using custom mapping function (see description with respect to FIG. 4 and further shown in FIG. 7) that transforms event Z-score to augmentation mean of the event;
      Event Z-score and variance were in turn sampled from empirical conditional distributions;
Advantageously, the data simulation is based on dimensionless Z-scores: allowing for the use of data with different protocols.

### Data Simulation Comparison

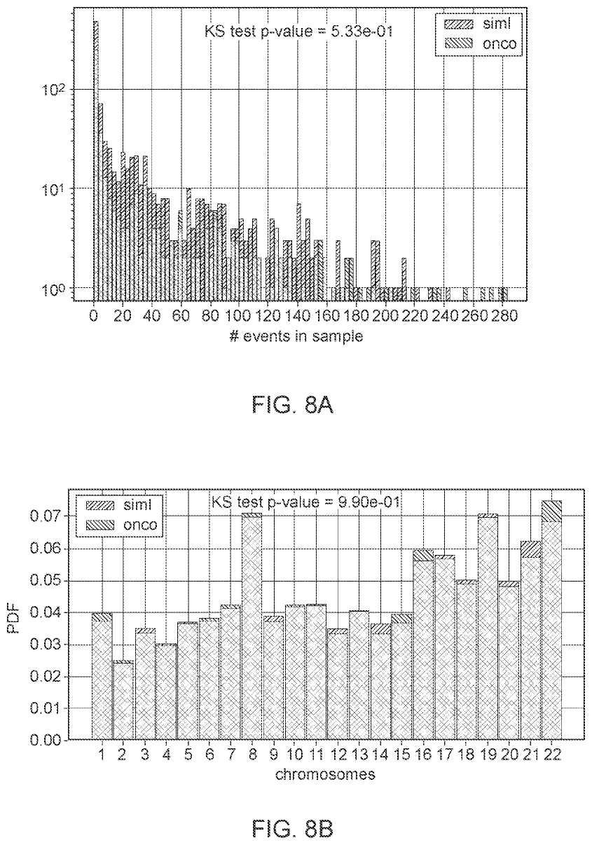

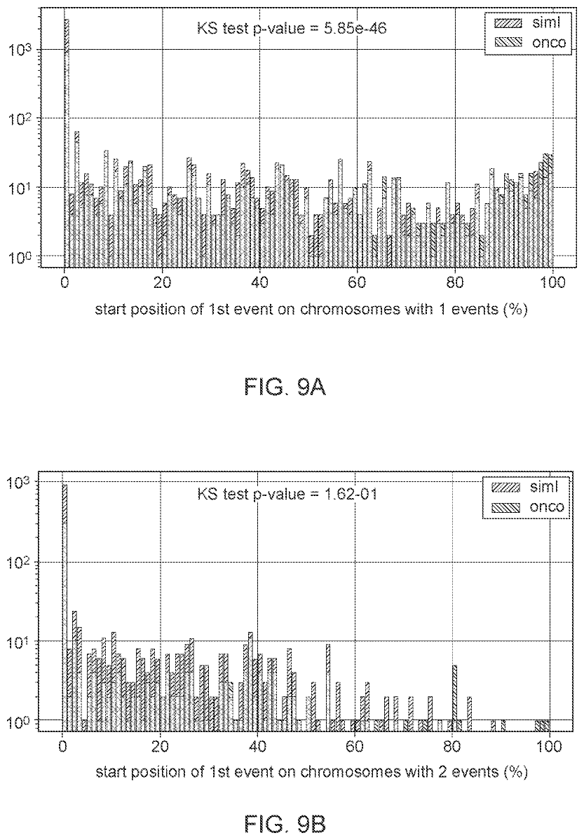

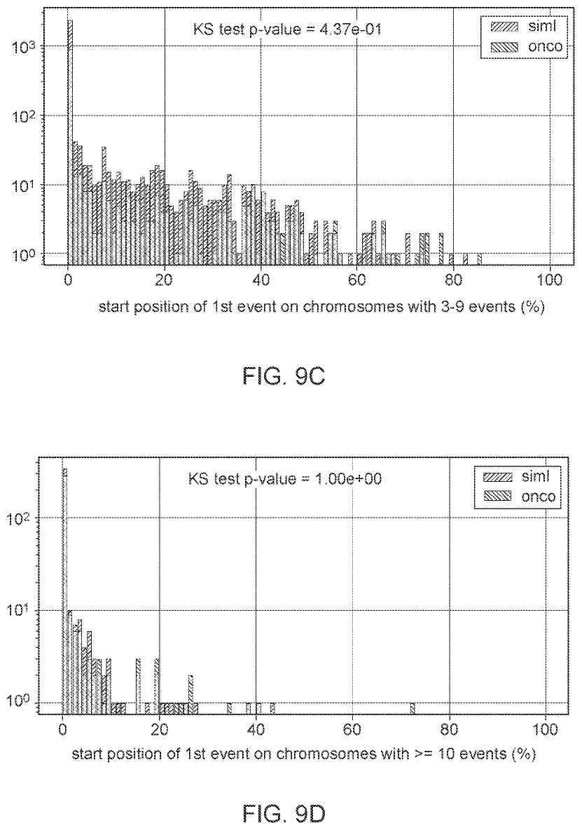

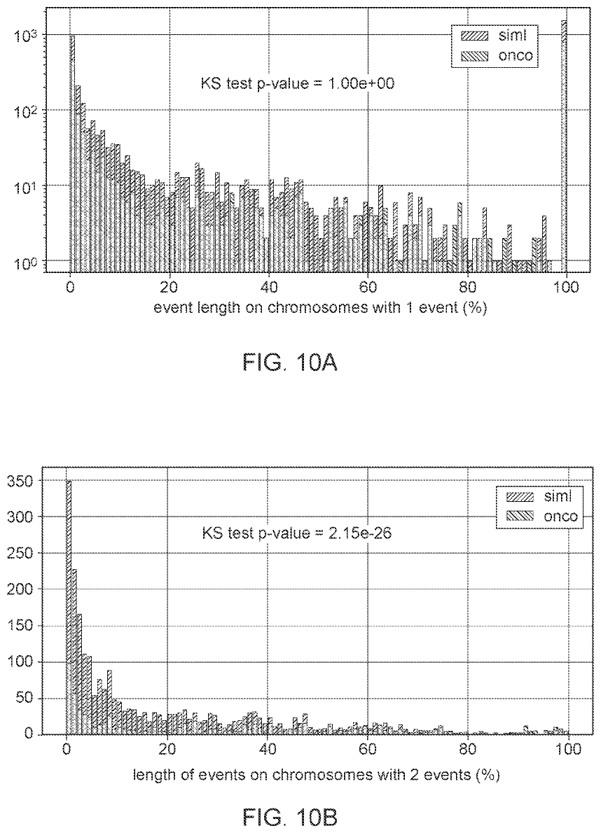

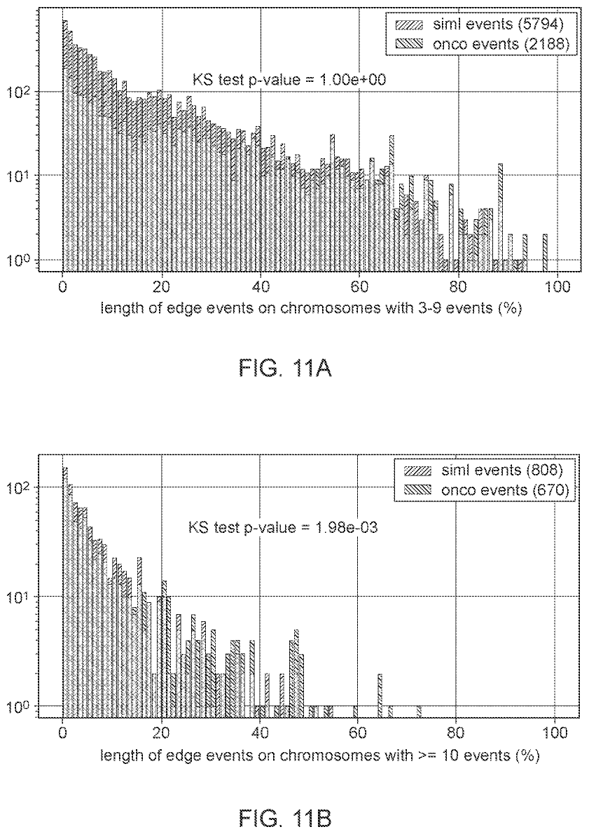

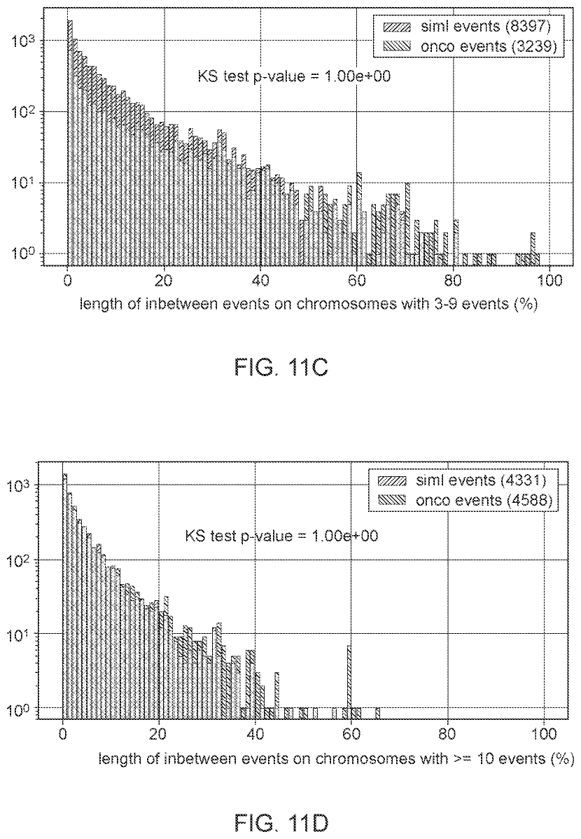

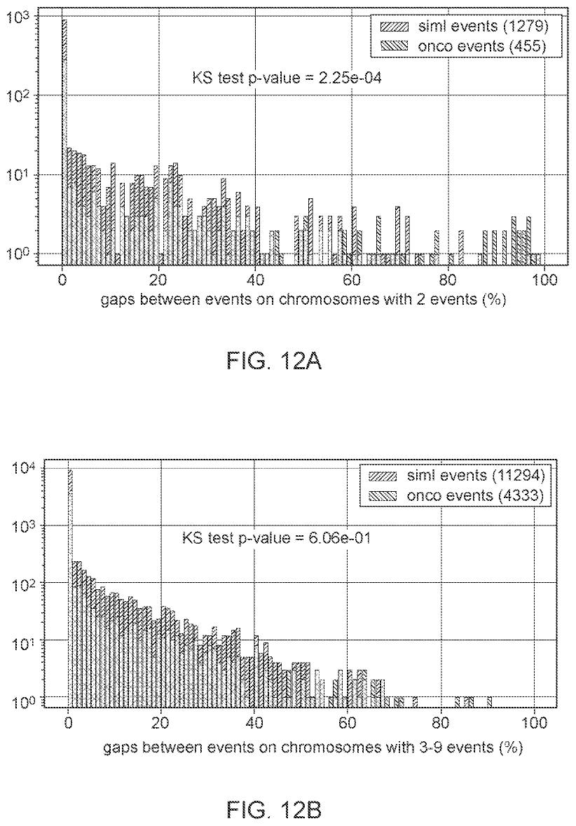

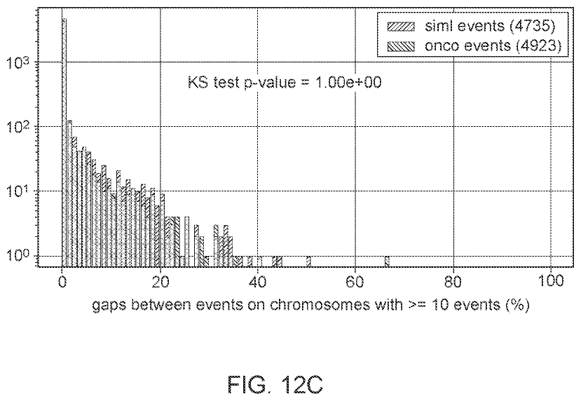

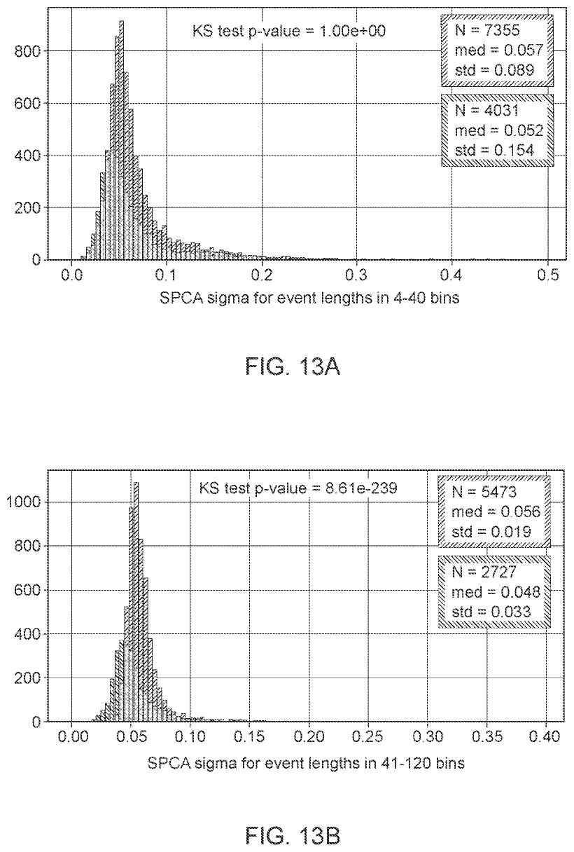

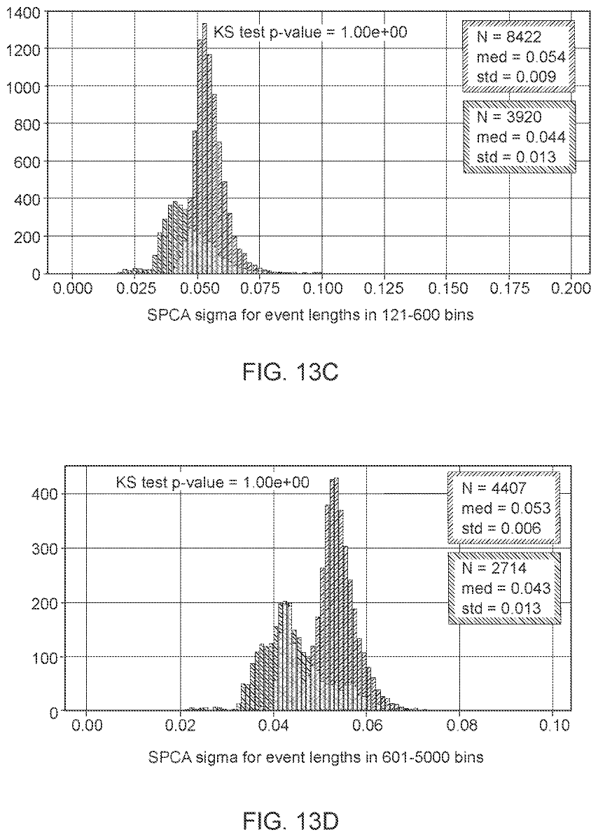

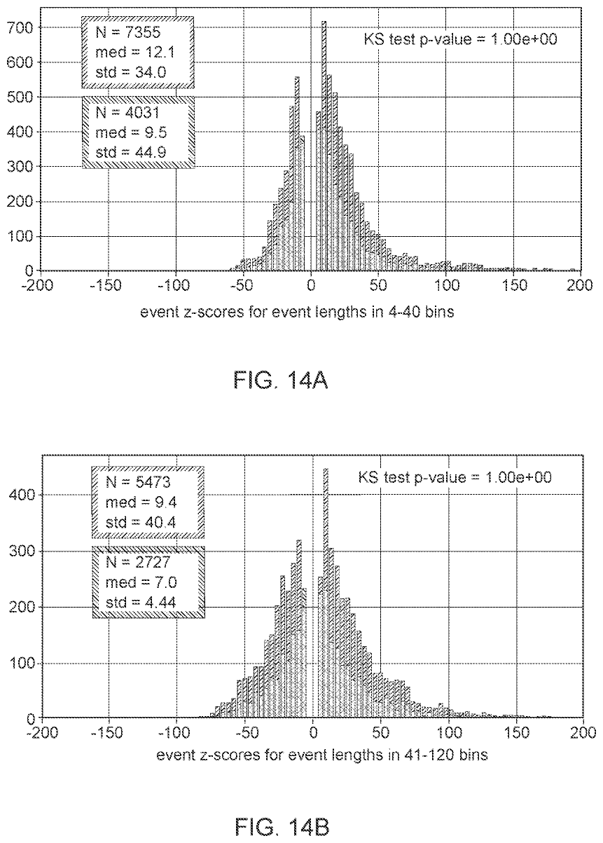

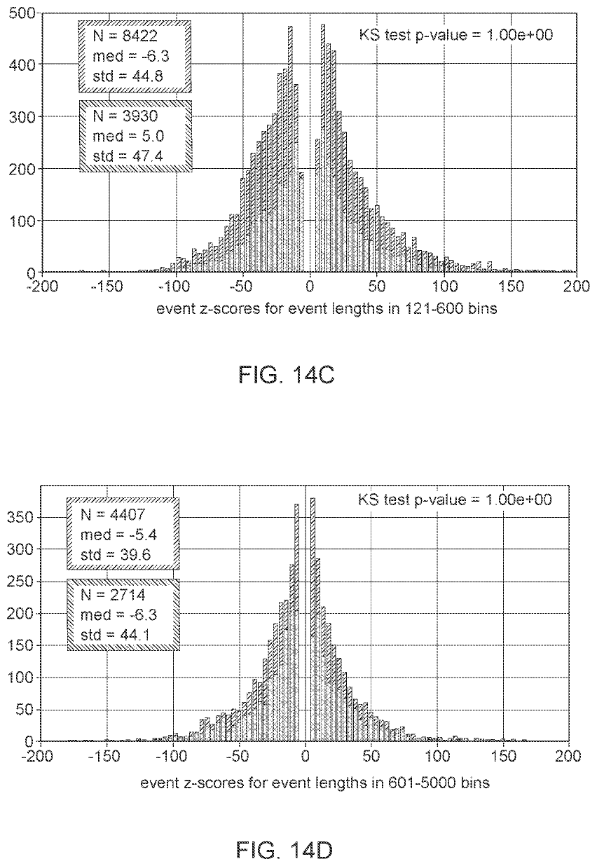

FIGS. 8A-14D show a comparison of CNV event characteristics distributions between the original population of cancer-positive samples and the population of simulated cancer-positive samples. The information is presented in the form of histograms or probability density function (PDF) profiles and corresponding estimates of p-values for Kolmogorov-Smirnov statistical test measuring equality of probability distributions. In general, if p-value is above a certain (arbitrary, e.g. 0.05 or 0.01) threshold, it is concluded that differences in probability distribution of a feature for samples comprising both datasets are not statistically significant and they belong to the same population. FIG. 8A shows a histogram of the number of events in a sample. FIG. 8B shows a PDF of chromosomes hosting events normalized by chromosome length. The number of events in a sample as well as distribution of events over chromosomes for real oncology and simulated oncology data came from a same population. As shown, the simulated oncology data is relatively comparable to the real oncology data in both number of events per sample and chromosomes hosting the various events. FIGS. 9A-9D show the start position of a 1st event on a chromosome for both the real oncology and simulated oncology data. FIGS. 10A-10B show event lengths on chromosomes with 1 and 2 events, respectively. FIGS. 11A-11D show event lengths on chromosomes with >=3 events. FIGS. 12A-12C show gaps between events on a single chromosome. FIGS. 13A-13D show SPCA standard deviation conditional on event length. The difference in standard deviation distribution between real oncology and simulated oncology data may be accounted for by the model assumptions described below. FIGS. 14A-14D show a comparison of event characteristic distributions. Z-scores for different event length groups for real oncology and simulated oncology data come from a same population.

### Experimental Setup

The original data set was comprised of about 3000 samples including 2000 oncology negative samples (NIPT samples) and 1000 simulated oncology positive samples. The 3000 samples were split into training and test subsets at 80/20 proportion. The training set was subjected to a 10-fold cross-validation (CV) and was used to compute the PCA object for feature transformation. The GIN values were computed for all samples and were used as the additional model feature. The logistic regression classifier was fit to each CV fold and tested on remaining 10% of the training set. The errors reported were specificity or true negative rate and sensitivity or true positive rate. The specificity was kept at a fixed level of 99%. The CV sensitivity was calculated as the average over all CV folds. The CV errors were used to find optimal set of model parameters, one of which is the number of principal components k to keep for the feature transformation procedure as follows:

```
          X ∈ [symbol]   ^{(m}^{^{×}}ⁿ⁾ - original data
              m ≈ 3000
            n ≈ 50000
        PC computation on train set:
      U^{k} = [u¹ ... u^{k}] ∈ [symbol]   ⁽ⁿ ^{^{<}} ^{k)} - first k PC
          PCA data transformation:
    Y^{k} = XU^{K} ∈ [symbol]   ^{(m} ^{^{∨}}^{k)} - transformed data
            k = [5, 40]
Y^{k} is used as a new training set to fit machine learning model
  3-4 orders of magnitude dimensionality reduction
```

The k values used fall in the range of [5, 40] and the results were reported for the instance where k=34. The additional subset of 10 oncology positive samples presents an opportunity for extra testing of model sensitivity.

### Results

The classification results in terms of specificities and sensitivities computed on the cross-validation and the independent test sets, and on the extra test set of oncology NIPT samples for different model configurations are presented in Table 1.

TABLE 1

| ML setup | Metric | CV | Test | Test extra |
|---|---|---|---|---|
| GIN | Specificity | 99 | | N/A |
| | Sensitivity | 60 | | 60 |
| Onco-gene features | Specificity | 99 | 99 | N/A |
| & GIN | Sensitivity | 67 | 65 | 50 |
| PCA-transformed | Specificity | 99 | 99 | N/A |
| features & GIN | Sensitivity | 87 | 82 | 80 |

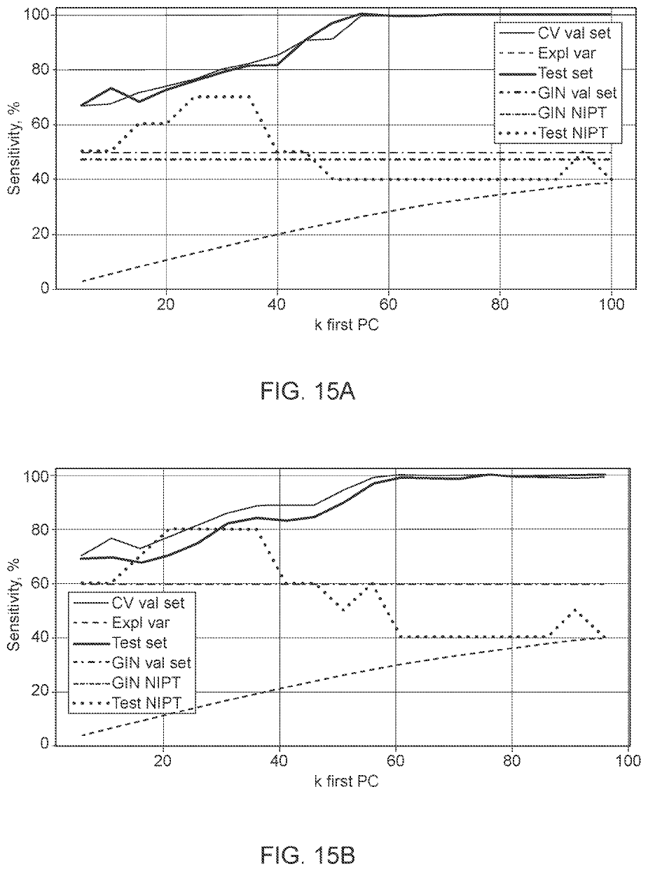

FIG. 15A shows the machine learning results of all positive and negative samples of MaterniT GENOME (results using the whole data set, CADET-negative and CADET-positive). FIG. 15B shows the machine learning results of only negative samples of MaterniT GENOME (k (# of PCA) in (20, 36) provides stable solution that outperforms benchmark method (GIN)). Table 2 summarizes the machine learning classification results.

TABLE 2

| | | | Test | | Test |
|---|---|---|---|---|---|
| Sample cohort | Metric | CV | (simulated) | GIN | (real data) |
| MaterniT | Specificity | 99 | 99 | 99 | 99 |
| GENOME | (%) | | | | |
| positive & | Sensitivity | 83 | 86 | 50 | 70 |
| negative | (%) | | | | |
| MaterniT | Specificity | 99 | 99 | 99 | 99 |
| GENOME | (%) | | | | |
| negative only | Sensitivity | 87 | 82 | 60 | 80 |
| | (%) | | | | |

The experimentation further demonstrated that the machine learning based application for pan-cancer screening is fast (runs under 1 min) and outperforms the existing method (GIN).

### Model Assumptions

The following assumption were made for implementation of the machine learning model:
Approximating sum of affected profile bin counts with sum of unaffected profile bin counts:

$${S}_{tot} = {\sum }_{i = 1}^{N} {y}_{i} \approx {\sum }_{i = 1}^{N} {y}_{i}^{b}$$

Approximating standard deviation of segment bin count augmentations with standard deviation of segment bin counts: Δσ_{seg}≈σ_{seg}
Assuming that mean of z-scores and bin count standard deviation is protocol-agnostic: mapping MaterniT® GENOME v4 data on v5 data
Assuming normal distribution of bin count augmentations in the affected CNV segment: [Δyₛₜ β Δyₑₙ]~ [symbol] (Δ̅y̅_{seg}, Δσ_{seg})

### VI. ADDITIONAL CONSIDERATIONS

Specific details are given in the above description to provide a thorough understanding of the embodiments. However, it is understood that the embodiments can be practiced without these specific details. For example, circuits can be shown in block diagrams in order not to obscure the embodiments in unnecessary detail. In other instances, well-known circuits, processes, algorithms, structures, and techniques can be shown without unnecessary detail in order to avoid obscuring the embodiments.

Implementation of the techniques, blocks, steps and means described above can be done in various ways. For example, these techniques, blocks, steps and means can be implemented in hardware, software, or a combination thereof. For a hardware implementation, the processing units can be implemented within one or more application specific integrated circuits (ASICs), digital signal processors (DSPs), digital signal processing devices (DSPDs), programmable logic devices (PLDs), field programmable gate arrays (FPGAs), processors, controllers, micro-controllers, microprocessors, other electronic units designed to perform the functions described above, and/or a combination thereof.

Also, it is noted that the embodiments can be described as a process which is depicted as a flowchart, a flow diagram, a data flow diagram, a structure diagram, or a block diagram. Although a flowchart can describe the operations as a sequential process, many of the operations can be performed in parallel or concurrently. In addition, the order of the operations can be re-arranged. A process is terminated when its operations are completed, but could have additional steps not included in the figure. A process can correspond to a method, a function, a procedure, a subroutine, a subprogram, etc. When a process corresponds to a function, its termination corresponds to a return of the function to the calling function or the main function.

Furthermore, embodiments can be implemented by hardware, software, scripting languages, firmware, middleware, microcode, hardware description languages, and/or any combination thereof. When implemented in software, firmware, middleware, scripting language, and/or microcode, the program code or code segments to perform the necessary tasks can be stored in a machine-readable medium such as a storage medium. A code segment or machine-executable instruction can represent a procedure, a function, a subprogram, a program, a routine, a subroutine, a subsystem, a software package, a script, a class, or any combination of instructions, data structures, and/or program statements. A code segment can be coupled to another code segment or a hardware circuit by passing and/or receiving information, data, arguments, parameters, and/or memory contents. Information, arguments, parameters, data, etc. can be passed, forwarded, or transmitted via any suitable means including memory sharing, message passing, ticket passing, network transmission, etc.

For a firmware and/or software implementation, the methodologies can be implemented with subsystems (e.g., procedures, functions, and so on) that perform the functions described herein. Any machine-readable medium tangibly embodying instructions can be used in implementing the methodologies described herein. For example, software codes can be stored in a memory. Memory can be implemented within the processor or external to the processor. As used herein the term “memory” refers to any type of long term, short term, volatile, nonvolatile, or other storage medium and is not to be limited to any particular type of memory or number of memories, or type of media upon which memory is stored.

Moreover, as disclosed herein, the term “storage medium”, “storage” or “memory” can represent one or more memories for storing data, including read only memory (ROM), random access memory (RAM), magnetic RAM, core memory, magnetic disk storage mediums, optical storage mediums, flash memory devices and/or other machine-readable mediums for storing information. The term “machine-readable medium” includes, but is not limited to portable or fixed storage devices, optical storage devices, wireless channels, and/or various other storage mediums capable of storing that contain or carry instruction(s) and/or data.

While the principles of the disclosure have been described above in connection with specific apparatuses and methods, it is to be clearly understood that this description is made only by way of example and not as limitation on the scope of the disclosure.

1. A computer-implemented method comprising:
accessing non-invasive prenatal testing (NIPT) sequence read data for a first group of samples, wherein the NIPT sequence read data was generated as part of performing a whole genome sequencing assay on NIPT samples, and the NIPT sequence read data includes bin count profiles comprising sequence read counts for each bin associated with a segment of a reference genome;
generating a first subset of training data and a second subset of training data based on the NIPT sequence read data, wherein each example in the second subset of training data is classified as negative for cancer;
accessing copy number variant data for a second group samples, wherein the copy number variant data was generated as part of performing a copy number variation assay, and the copy number variant data includes information on cancer patient derived genomic events, which are copy number counts for one or more segments that deviate from a reference profile;
generating a set of synthetic training data based on the copy number variant data obtained from cancer patients, wherein generating the set of synthetic training data comprises generating an empirical population of the genomic events characteristic of the copy number variant data obtained from the cancer patients, extracting event features and standard scores from the empirical population of the genomic events, and mapping the event features and standard scores to the bin count profiles for the second subset of training data to generate the set of synthetic training data, and wherein each example in the set of synthetic training data is classified as positive for cancer;
augmenting the first subset of training data with the set of synthetic training data to generate an augmented set of training data; and
training, using the augmented set of training data, a machine learning model to classify a sample as being negative for cancer or positive for cancer, wherein the training comprises: iterative operations to find a set of parameters for the machine learning model that minimizes a loss or error function for the machine learning model, wherein each iteration includes finding the set of parameters for the machine learning model so that a value of the loss or error function using the set of parameters is smaller than a value of the loss or error function using another set of parameters in a previous iteration, wherein the loss or error function is configured to measure a difference between (i) class outputs inferred for each example in the augmented set of training data, and (ii) labels that provide ground truth information for each example in the augmented set of training data, and wherein the ground truth information identify whether the example is classified as negative for cancer or classified as positive for cancer.

2. The computer-implemented method of claim 1, wherein generating the first subset of training data and the second subset of training data comprises normalizing the NIPT sequence read data by: (i) smoothing the bin count profiles using LOESS, (ii) population-based correction of the bin count profiles using principal component analysis, or (iii) both.

3. The computer-implemented method of claim 1, further comprising reducing dimensionality of the augmented set of training data using (i) another principal component analysis, wherein the other principal component analysis comprises mapping the augmented set of training data on first k principal components while discriminating between negative and positive classes, which reduces data redundancy and reduces a feature space n, or (ii) an onco-gene feature selection process, wherein the onco-gene feature selection process comprises generating a subset of predetermined number b of bins overlapped with a predetermined number o of onco-genes and use the bin count profiles from the normalized NIPT sequence read data at the subset of predetermined number b of bins as new model attributes.

4. (canceled)

5. The computer-implemented method of claim 1, further comprising:
determining an indicator of systematic abnormality in each example of the augmented set of training data, wherein the indicator is a genome instability number, a quantity of tumor content, or both; and
appending the indicator of systematic abnormality to each respective example of augmented set of training data,
wherein the machine learning model is trained, using the augmented set of training data with the appended indicator of systematic abnormality, to classify the sample as being negative for cancer or positive for cancer, and wherein the machine learning model uses the indicator of systematic abnormality as an additional model feature during training to find the set of parameters.

6. The computer-implemented method of claim 5, wherein the genome instability number is calculated as an integral of absolute deviations of a LOESS-smoothed normalized autosomal bin count profile from expectation.

7. The computer-implemented method of claim 1, wherein the set of synthetic training data presents a same statistical distribution of genomic events as presented in the copy number variant data.

8. A computer-implemented method comprising:
accessing non-invasive prenatal testing (NIPT) sequence read data for a sample, wherein the sequence read data was generated as part of performing a whole genome sequencing assay on the sample, and the NIPT sequence read data includes a bin count profile comprising sequence read counts for each bin associated with a segment of a reference genome;
determining an indicator of systematic abnormality for the sample based on the NIPT sequence read data, wherein the indicator is a genome instability number, a quantity of tumor content, or both; and
inputting the NIPT sequence read data and the indicator of systematic abnormality into the machine learning model trained using the method of claim 1;
classifying, using the machine learning model, the sample as being a negative class or positive class for cancer, wherein the machine learning model uses the indicator of systematic abnormality as an additional model feature for predicting the negative class or the positive class; and
outputting, using the machine learning model, the negative class or the positive class for cancer.

9.-11. (canceled)

12. The computer-implemented method of claim 8, further comprising generating a report that includes the negative class or the positive class for cancer and a result of the whole genome sequencing assay.

13. A system comprising:
one or more processors; and
one or more computer-readable media storing instructions which, when executed by the one or more processors, cause the system to perform operations comprising:
accessing non-invasive prenatal testing (NIPT) sequence read data for a first group of samples, wherein the NIPT sequence read data was generated as part of performing a whole genome sequencing assay on NIPT samples, and the NIPT sequence read data includes bin count profiles comprising sequence read counts for each bin associated with a segment of a reference genome;
generating a first subset of training data and a second subset of training data based on the NIPT sequence read data, wherein each example in the second subset of training data is classified as negative for cancer;
accessing copy number variant data for a second group samples, wherein the copy number variant data was generated as part of performing a copy number variation assay, and the copy number variant data includes information on cancer patient derived genomic events, which are copy number counts for one or more segments that deviate from a reference profile;
generating a set of synthetic training data based on the copy number variant data obtained from cancer patients, wherein generating the set of synthetic training data comprises generating an empirical population of the genomic events characteristic of the copy number variant data obtained from the cancer patients, extracting event features and standard scores from the empirical population of the genomic events, and mapping the event features and standard scores to the bin count profiles for the second subset of training data to generate the set of synthetic training data, and wherein each example in the set of synthetic training data is classified as positive for cancer;
augmenting the first subset of training data with the set of synthetic training data to generate an augmented set of training data; and
training, using the augmented set of training data, a machine learning model to classify a sample as being negative for cancer or positive for cancer, wherein the training comprises: iterative operations to find a set of parameters for the machine learning model that minimizes a loss or error function for the machine learning model, wherein each iteration includes finding the set of parameters for the machine learning model so that a value of the loss or error function using the set of parameters is smaller than a value of the loss or error function using another set of parameters in a previous iteration, wherein the loss or error function is configured to measure a difference between (i) class outputs inferred for each example in the augmented set of training data, and (ii) labels that provide ground truth information for each example in the augmented set of training data, and wherein the ground truth information identify whether the example is classified as negative for cancer or classified as positive for cancer.

14. The system of claim 13, wherein generating the first subset of training data and the second subset of training data comprises normalizing the NIPT sequence read data by: (i) smoothing the bin count profiles using LOESS, (ii) population-based correction of the bin count profiles using principal component analysis, or (iii) both.

15. The system of claim 13, wherein the one or more computer-readable media further store instructions which, when executed by the one or more processors, cause the system to perform operations comprising reducing dimensionality of the augmented set of training data using (i) another principal component analysis, wherein the other principal component analysis comprises mapping the augmented set of training data on first k principal components while discriminating between negative and positive classes, which reduces data redundancy and reduces a feature space n, or (ii) an onco-gene feature selection process, wherein the onco-gene feature selection process comprises generating a subset of predetermined number b of bins overlapped with a predetermined number o of onco-genes and use the bin count profiles from the normalized NIPT sequence read data at the subset of predetermined number b of bins as new model attributes.

16. (canceled)

17. The system of claim 13, wherein the one or more computer-readable media further store instructions which, when executed by the one or more processors, cause the system to perform operations comprising:
determining an indicator of systematic abnormality in each example of the augmented set of training data, wherein the indicator is a genome instability number, a quantity of tumor content, or both; and
appending the indicator of systematic abnormality to each respective example of augmented set of training data,
wherein the machine learning model is trained, using the augmented set of training data with the appended indicator of systematic abnormality, to classify the sample as being negative for cancer or positive for cancer, and wherein the machine learning model uses the indicator of systematic abnormality as an additional model feature during training to find the set of parameters.

18. The system of claim 17, wherein the genome instability number is calculated as an integral of absolute deviations of a LOESS-smoothed normalized autosomal bin count profile from expectation.

19. The system of claim 13, wherein the set of synthetic training data presents a same statistical distribution of genomic events as presented in the copy number variant data.

20.-24. (canceled)

25. A computer-program product tangibly embodied in a non-transitory machine-readable medium, including instructions configured to cause one or more data processors to perform operations comprising:
accessing non-invasive prenatal testing (NIPT) sequence read data for a first group of samples, wherein the NIPT sequence read data was generated as part of performing a whole genome sequencing assay on NIPT samples, and the NIPT sequence read data includes bin count profiles comprising sequence read counts for each bin associated with a segment of a reference genome;
generating a first subset of training data and a second subset of training data based on the NIPT sequence read data, wherein each example in the second subset of training data is classified as negative for cancer;
accessing copy number variant data for a second group samples, wherein the copy number variant data was generated as part of performing a copy number variation assay, and the copy number variant data includes information on cancer patient derived genomic events, which are copy number counts for one or more segments that deviate from a reference profile;
generating a set of synthetic training data based on the copy number variant data obtained from cancer patients, wherein generating the set of synthetic training data comprises generating an empirical population of the genomic events characteristic of the copy number variant data obtained from the cancer patients, extracting event features and standard scores from the empirical population of the genomic events, and mapping the event features and standard scores to the bin count profiles for the second subset of training data to generate the set of synthetic training data, and wherein each example in the set of synthetic training data is classified as positive for cancer;
augmenting the first subset of training data with the set of synthetic training data to generate an augmented set of training data; and
training, using the augmented set of training data, a machine learning model to classify a sample as being negative for cancer or positive for cancer, wherein the training comprises: iterative operations to find a set of parameters for the machine learning model that minimizes a loss or error function for the machine learning model, wherein each iteration includes finding the set of parameters for the machine learning model so that a value of the loss or error function using the set of parameters is smaller than a value of the loss or error function using another set of parameters in a previous iteration, wherein the loss or error function is configured to measure a difference between (i) class outputs inferred for each example in the augmented set of training data, and (ii) labels that provide ground truth information for each example in the augmented set of training data, and wherein the ground truth information identify whether the example is classified as negative for cancer or classified as positive for cancer.

26. The computer-program product of claim 25, wherein generating the first subset of training data and the second subset of training data comprises normalizing the NIPT sequence read data by: (i) smoothing the bin count profiles using LOESS, (ii) population-based correction of the bin count profiles using principal component analysis, or (iii) both.

27. The computer-program product of claim 25, further including instructions configured to cause the one or more data processors to perform operations comprising reducing dimensionality of the augmented set of training data using (i) another principal component analysis, wherein the other principal component analysis comprises mapping the augmented set of training data on first k principal components while discriminating between negative and positive classes, which reduces data redundancy and reduces a feature space n, or (ii) an onco-gene feature selection process, wherein the onco-gene feature selection process comprises generating a subset of predetermined number b of bins overlapped with a predetermined number o of onco-genes and use the bin count profiles from the normalized NIPT sequence read data at the subset of predetermined number b of bins as new model attributes.

28. (canceled)

29. The computer-program product of claim 25, further including instructions configured to cause the one or more data processors to perform operations comprising:
determining an indicator of systematic abnormality in each example of the augmented set of training data, wherein the indicator is a genome instability number, a quantity of tumor content, or both; and
appending the indicator of systematic abnormality to each respective example of augmented set of training data,
wherein the machine learning model is trained, using the augmented set of training data with the appended indicator of systematic abnormality, to classify the sample as being negative for cancer or positive for cancer, and wherein the machine learning model uses the indicator of systematic abnormality as an additional model feature during training to find the set of parameters.

30. The computer-program product of claim 29, wherein the genome instability number is calculated as an integral of absolute deviations of a LOESS-smoothed normalized autosomal bin count profile from expectation.

31. The computer-program product of claim 25, wherein the set of synthetic training data presents a same statistical distribution of genomic events as presented in the copy number variant data.

32.-36. (canceled)
